(19) United States
(12) Patent Application Publication
(10) Pub. No.: US 2006/0116331 A1
(43) Pub. Date: Jun. 1, 2006

(54) Glycosylceramide analogues

(75) Inventors: Zi-Hua Jiang, Edmonton (CA); Sham Gandhi, Edmonton (CA); R. Rao Koganty, Edmonton (CA)

Correspondence Address: BROWDY AND NEIMARK, P.L.L.C.;624 NINTH STREET, NW, SUITE 300, WASHINGTON, DC, 20001-5303, US

(73) Assignee: BIOMIRA, INC., Edmonton, Alberta (CA)

(21) Appl. No.: 10/529,393

(22) PCT Filed: Sep. 29, 2003
(86) PCT No.: PCT/US03/30611

§ 371 Date: Jan. 31, 2006

Related U.S. Application Data
(60) Provisional application No. 60/413,882, filed on Sep. 27, 2002.

(51) Int. Cl. A61K 31/704; C07J 17/00; C07H 15/24

(52) U.S. Cl. 514/25; 514/169; 514/26; 514/33; 536/6.1

(57) ABSTRACT

Glycosylceramide analogues are disclosed in which the ceramide moiety and optionally the carbohydrate moiety are modified or replaced. These analogues are useful as immunomodulators, antitumor agents, and as other pharmaceutical agents.

This application claims the benefit of Gandhi et al., U.S. Prov. Appl. No. 60/413,882, filed Sep. 27, 2002, and hereby incorporated by reference in its entirety.

### BACKGROUND OF THE INVENTION

1. Field of the Invention

The present invention relates to novel glycolipids which have biological activity, e.g., the ability to modulate the immune system. More specifically, synthetic analogues of α-galactosylceramides are disclosed. These molecules have the potential to activate the immune cells by inducing the secretion of cytokines and modulate immune responses. The invention also relates to the therapeutic application of these molecules in immunotherapy, in particular as immunostimulatory adjuvants for vaccine development and as immunoinhibitory agents for the treatment of autoimmune diseases and inflammation.

2. Description of the Background Art

As its name suggests, a glycosylceramide combines a carbohydrate moiety and a ceramide moiety. A ceramide, in turn, comprises the divalent residue of a sphingoid base (a long-chain aliphatic amino alcohol), and a monovalent fatty acyl moiety. More particularly, it is the result of acylating the amino nitrogen of the divalent residue (—O—CH2-CH(—NH—)—R′) of a sphingoid base to obtain —O—CH2-CH(—NH-R″)—R′ (where R′ is alkyl or alkenyl, and may be hydroxylated, and where R″ is a fatty acyl group, —C(═O)—R^{a}, where R^{a} is substituted or unsubstituted alkyl). The galactosylceramide is thus the result of O-linking the Galactose to the residue of the ceramide, i.e.,

Galactose-O—CH2-CH(—NH-R″)-R′

Galactosylceramides are the principal glycosphingolipids in brain tissue, and hence are also known as cerebrosides. Glucosylceramides are the principal glycosphingolipids in the photosynthetic tissues of plants. They are also found in animal tissues, for example, in skin lipids. Other glycosylceramides are known in nature.

The naturally occurring sphingoid bases vary in terms of the length of the main carbon chain (usually 14-22 carbons), the number of double bonds (usually 0, 1, or 2; the double bonds may be cis or trans, and the location(s) can vary, e.g., C-4 in sphingosine and C-8 in dehydrophytosphingosine), and the number of hydroxyl groups (usually 2 or 3; note that in a galactosylceramide, one of these hydroxyl groups becomes —OR, where R is the Gal). They can have branched chains, e.g., with methyl substituents. Much if not all of this variation is also seen among the naturally occurring glycosylceramides.

Among the naturally occurring ceramides, there is also variation in the length of the fatty acid moiety (usually 16-26, with some preference for even numbers), and in whether or not the fatty acid moiety is hydroxylated.

Agelasphins, a family of α-galactosylceramides (α-GalCer, FIG. 1), were originally extracted from marine sponges and found to exhibit potent anti-tumor properties and other therapeutic applications (Natori et al. 1994). One of α-GalCer's synthetic analogues, KRN7000 (FIG. 1; compound 7 in FIG. 11) is a promising immunomodulatory agent, which is currently being evaluated for its potential benefits in antitumor and antiinfectious therapies as well as in the prevention of type I diabetes and autoimmune encephalomyelitis. The adjuvant effect of α-GalCer has also been demonstrated with various different immunogens by its ability to strongly enhance antigen-specific CD8⁺ T cell response (Gonzalez-Aseguinolaza et al. 2002).

Peptide/glycopeptide antigens are processed and presented by antigen presenting cells (APC) in the context of MHC I or II to T cell receptors (TCRs). On the other hand, glycolipid antigens are bound to CD1 molecules and presented to TCR. CD1 molecules represent a new class of highly conserved, antigen presenting cell surface proteins (Park, S.-H. & Bendelac, A. Nature, 2000, 406, 788-792). They recognize and bind glycolipid antigens through lipid -protein interactions and present the sugar moiety of the antigen to a receptor on natural killer T-cells (NKT cells) to activate the immune system. In humans, five different isoforms of CD1 have been detected so far. In the case of α-GalCer, it binds to CD1d molecule and the complex is recognized at picomolar concentrations by the conserved semi-invariant, CD1d-restricted αb TCR of mouse and human NKT cells (Kawano et al. 1997). The nature and orientation of the polar head group of α-GalCer molecule are likely to be important for TCR contact, while the nature of the lipophilic group in the ceramide moiety modulates the binding of α-GalCer to CD1d molecule.

α-GalCer and its analogues are known to induce cell proliferation and cytokine production by natural killer (NK) T cells. Recently it was demonstrated that activation of NK T cells by α-GalCer causes bystander activation of NK, B, CD4⁺, and CD8⁺ T cells (Gonzalez-Aseguinolaza et al. 2002). A unique property of α-GalCer is its ability to induce both Th1 and Th2 immunity, which in turn is effected by cytokines, e.g., interleukin-4 (IL-4) and interferon-gamma (IFN-γ). Some α-GalCer analogues elicit substantial amount of both IL-4 and IFN-g, while others elicit one predominantly over the other. It is well understood in immunology that IL-4 supports humoral immune (Th2) responses, while IFN-γ supports cellular immune (Th1) response. Compounds that elicit predominantly or exclusively IL-4 might be useful as therapeutic agents for Th1-mediated autoimmune diseases, such as inflammation, type I diabetic, and multiple sclerosis. On the other hand, compounds that predominantly elicit IFN-γ might be useful in effective vaccine development against intra-cellular pathogens, such as malaria, tuberculosis, and cancers.

α-GalCer is a glycolipid comprising a hydrophilic carbohydrate moiety with α-linkage to the hydrophobic ceramide portion consisting of a long fatty acyl chain (C₂₆) N-linked to sphingosine base (C₁₈). Molecular interaction of α-GalCer with CD1d is necessary for Vα14 NKT cell activation. It is speculated that the ceramide portion binds to the floor of the hydrophobic cleft of CD1d, while the hydrophilic sugar moiety is likely to interact with the Vα14/Vb8.2 receptor and/or α-helix of CD1d. Structure-activity relationship studies (Uchimura, A. et al. Bioorg. Med. Chem. 1997, 5, 1447; Uchimura, A. et al. Bioorg. Med. Chem. 1997, 5, 2245-2249; Costantino, V. et al. Tetrahedron, 1996, 52, 1573-1578; Morita, M. et al. J. Med. Chem. 1995, 38, 2176-2187; Kawano et al., Science, 1997, 278, 1616-1629) have shown that,
the length of the carbon chains on the ceramide is important, because a shorter length of either the fatty acyl chains or the sphingosine base reduced its ability to cause Vα14 NKT cell proliferation;
the α-anomeric configuration of the inner sugar is very important for stimulation of Vα14 NKT cells, as indicated by the fact that β-GalCer does not stimulate Vα14 NKT cells readily; in addition, many kinds of monoglycosylated β-D-pyranosylceramides (lactosylceramide, etc.) occur naturally, but there is no report that these monoglycosylated β-D-pyranosylceramides have marked immunostimulatory effects;
the configuration of the 2-OH group of the sugar moiety is very important for stimulation of Vα14 NKT cells because α-mannosylceramide (α-ManCer), having a different configuration of the 2-OH group of the sugar moiety from α-CalCer, failed to stimulate Vα14 NKT cells;
the configuration of the 4-OH of the sugar moiety is not important for the manifestation of NKT immunostimulatory activity, since α-glucosylceramide (α-GlcCer) readily stimulate Vα4 NKT cells;
the configuration of 6-OH group of the sugar moiety is less important for the manifestation of the NKT immunostimulatory activity; and
the 3′-OH on the sphingosine is very important for NKT immunostimulatory activity, because α-GalCer lacking 3′-OH sphingosine has no effect.

Collectively, both carbohydrate and ceramide moieties play important roles in the exhibition of biological activities of α-GalCer molecules. Since the recognition event is highly specific for glycolipids and no carrier proteins are required, this novel defense mechanism has gained considerable interest in the past years, with the hope that a new type of therapeutic agents, including vaccines, may be developed in the future. With our growing knowledge of how α-GalCers stimulate immune cells, our current interest focuses on the discovery of novel synthetic analogues of α-GalCer with biological activities similar to their natural counterparts. One specific interest is to design novel structures which can elicit predominantly Th2 cytokine(s), e.g. (IL-4), over Th2 cytokine(s), e.g. IFN-γ, or vise versa, so that selective therapeutic benefits can be found with these compounds based on their ability of inducing different cytokine profiles.

Glycosylceramides with unsaturated fatty acyl moieties. Costantino, et al., Bioorgan. Med. Chem. Lett. 9: 271-6 (1999) discloses two glycosyl ceramides (compounds 2a and 2b, named plakoside A and B) in which the fatty acyl moiety (corresponding to R3 in our formula F-A) comprises a single alkenic double bond. Plakoside A and B were isolated from the Caribbean sponge Plakortis simplex. These “simplexides” are immunoinhibitory agents.

Glycosylceramides are also known which have unsaturated sphingoid base moieties. The website www.lipid.co.uk/infores/Lipids/cmh refers to the existence of cerebrosides of seeds from scarlet runner beans and kidney beans whose sphingoid bases have the structures d18:2-4t,8t or d18:2-4t,8c.

Glycosylceramide analogues with steroidal, terpenoidal or alkaloidal moieties. We are not aware of any naturally occurring or synthetic glycosylceramide analogues with steroidal, terpenoidal or alkaloidal moieties. In this regard, it should be noted that while AGL-597 contains biotin (AGL597, the biotinylated analogue of KRN7000, was reported by Sakai, et al., Organic Lett. 1: 359-61 (1999) ), and biotin contains heterocyclic nitrogen, we do not believe that the art normally identifies biotin as an alkaloid. However, to avoid any possibility of confusion, we have defined “alkaloid” to formally exclude biotin.

Fluorinated glycosylceramide analogues. Fluorine occurs extremely rarely in biomolecules, mostly as a monofluorinated fatty acid, at the omega carbon.

Fluorocarbons share many of the properties of the cognate hydrocarbons. For example, fluorinated analogs of natural compounds can still be recognized by the normal enzymes or receptors. Thus, fluorinated methylmethionine, tryptophan, phenylalanine and tyrosine are still recognized by cognate amino acyl-tRNA synthetases. See Marsh, E. Neil G., “Toward the nonstick egg: designing fluorous proteins”, Chemistry & Biology 7:R153-R157 (2000). Indeed, fluorination can increase binding; trifluoroleucine syubstitution in melittin had enhanced affinity for lipid bilayer membranes. Niemz and Tirrell, “Self-association and membrane-binding behavior of melittins containing trifluoroleucine”, J. Am. Chem. Soc. 123: 7407-13 (2001).

The fluorocarbons are, however, much more hydrophobic than their cognate hydrocarbons. For example, trifluoromethyl is over twice as hydrophobic as methyl. Fluorination has been used to increase the lipophilicity, and hence bioavailability of drugs, as in the case of fenfluramine. However, while some fluorocarbons are hydrophobic, perfluorocarbons are poorly soluble in hydrocarbon solvents, leading one commenter to refer to them as being fluorophilic, rather than lipophilic. The synthesis of fluorous proteins has been suggested. See Marsh (2000).

Faroux-Corlay, et al., “Synthesis of single- and double-chain fluorocarbon and hydrocarbon galactosyl amphiphiles and their anti-HIV-1 activity”, Carbohydr. Res., 327: 223-260 (2000), describes the synthesis of three series of fluorinated analogues of beta GalCer, and evaluation of their anti-HIV activity. Beta GalCer is an alternative receptor allowing HIV-1 entry into CD4(−)/GalCer(+) cells by recognition of the V3 loop of HIV gp120.

In the first series, in the terms of our general formula A, R is beta-Gal, L is the native —CH2-CH<, R2 is H, and A′ and R3 are as follows:

| A′ | R3 (their R2) |
|---|---|
| —C(═O)—NH—(CH2)13CH3 | —C(═O) (CH2)10C4F9 |
| —C(═O)—NH—(CH2)15CH3 | —C(═O) (CH2)10C6F13 |
| —C(═O)—NH—(CH2)11C4F9 | —C(═O) (CH2)10C6F13 |

In the second series, the group corresponding to R3 in our general formula F-A′ is —C(═O) (CH2)4C6F13, while R2 is —(CH2)24-N(—C(═O)R3)-CH2CH2OH or —(CH2)24-N(—C(═O)R3)-CH2CH2O— betaGal, R is betaGal, L is —CH2-CH<, and A′ is —H. (Note that we do not allow all of these choices.)

Finally, in the third series, the fluorinated analogue is one corresponding to our general formula I-A′ in which R3 is —C(═O) (CH2)6C8F17, R2 is —(CH2)15CH3, R is beta Gal, L is —CH2-CH<, and A′ is —H.

In each series, the fluorocarbon analogue had greater anti-HIV activity than the hydrocarbon cognate. See also Faroux-Corlay et al., “Amphiphilic anionic analogues of galactosylceramide: synthesis, anti-HIV-1 activity, and gp120 binding,” J. Med. Chem., 44: 2188-2203 (2001); Clary, et al., “Synthesis of single- and double-chain fluorcarbon and hydrocarbon β-linked galactose amphiphiles derived from serine,” Tetrahedron Lett., 36: 539-42 (1995).

Miscellaneous. The following patents relate to therapeutic use of ceramides or ceramide analogues and may be of interest: Motoki, U.S. Pat. No. 6,555,372; Taniguchi, U.S. Pat. No. 6,531,453; Longwood, U.S. Pat. No. 6,103,883; Shayman, U.S. Pat. No. 6,569,889; Maruyama, U.S. Pat. No. 6,417,167.

Pentaerythritol. Pentaerythritol (Pet) and di-pentaerythritol (di-Pet) are common polyols and they are widely used in oil industry to produce lubricants and other macromolecules. A derivative, tetrakis-[13-(2′-deoxythymidin-3′-O-yl)-6,9-diaza-2-oxa-5,10,13-trioxotridecyl)-methane (dT₄-PE-PLC) has been used as a liquid phase carrier for large-scale oligonucleotide synthesis in solution. In addition, Pet derivatives, semifluorinated pentaerythritol tetrabenzoates, have been employed to design liquid crystalline structures (Cheng, X. H. et al, 2000) and pentaerythritol lipid derivatives (e.g., dimristoyl-trimethylglycine pentaerythritol) have been used in the preparation of cationic liposomes for the delivery of nucleic acids into mammalian cells. A triamine derivative of pentaerythritol has been used as a starting material in the preparation of chelating agents.

The four-directional core (the “Pet” unit) of pentaerythritol has been employed successfully as a coupling agent, for example, in the synthesis of multifunctional dendrimers (Armspach, D. et al, 1996 and Kuzdzal, S. A. et al, 1994), and as a molecular scaffold for combinatorial chemistry (Farcy, N. et al, 2001).

It is particularly interesting to note the use of the Pet unit to couple sugar units. Lindhorst, et al, Eur. J. Org. Chem., 2027-34 (2000) used the Pet unit as a framework for a cluster of four mannosides. Schmidt, et al., Eur. J. Org. Chem., 669-674 (2002) prepared similar structures in which a lipid group (C16H33) was O-linked to one of the four peripheral carbons, and one to three mannoside residues were O-linked, through an ethyleneoxy oligomeric spacer, to other of the peripheral carbons. Those peripheral carbons which did not link to a lipid or to a sugar-containing moiety were simply hydroxylated. Finally, Hanessian et al. 1996 used a pentaerythritol scaffold to present a cluster of two Tn (the monosaccharide GalNAc) or TF (the disaccharide D-Galβ(1->3)GalNAc) epitopes, each O-linked through a spacer to a peripheral carbon of the Pet core. Of remaining two peripheral carbons, one was O-linked to —CH2CH2NHAc, and the other O-linked to either allyl (Hanessian 33) or 1-octenyl (Hanessian 37). In none of these references was a peripheral carbon of the Pet core N-linked to any moiety.

In the various applications mentioned above, the Pet unit serves as a core to carry other moieties. It may also be used to replace a sugar unit in an oligosaccharide.

Toepfer et al disclosed sialyl-Lewis X and sialyl-Lewis A mimics containing one Pet unit (Toepfer et al. 1995; Toepfer et al. 2000) as new ligands for selectin binding. Thus, in compound 4 of Toepfer et al. 1995, two of the peripheral carbons of the Pet unit are hydroxylated, one is O-linked to a moiety comprising a single sugar unit, and the last one is O-linked to a moiety comprising a disaccharide. It should be noted that in Toepfer's analogs, the Pet unit replaces a normal sugar unit, not an amino sugar. In addition, the only lipophilic groups contemplated by Toepfer et al. are groups customarily used as protecting groups in organic synthesis, such as those resulting in replacement of sugar hydroxyls with —O-All, —O-Tf, or —O-Bn.

Aguilera et al. 1988 reported the testing of analogs of oligosaccharides for anti-mitotic activity. The original oligosacccharides were the tetrasaccharide α-D-GalNac-β-D-Gal-(1→4)-[α-L-Fuc-(1→3)]-β-D-GlcOMe, and a related sulfated trisaccharide (Aguilera compound 1), which contain a Lewis X-type structure. In the analogs of the trisaccharide (Aguilera compounds 13-16), one sugar was replaced with a Pet unit. In the analogs of the tetrasaccharide (17, 18), two of the sugar units were replaced with Pet units. The analogs thus contained the disaccharide in which the α-fucosyl residue was linked to the C-3 position of the GlcNac. In all six analogs, one hydroxyl of the disaccharide moiety was replaced with —O(CH₂)₇CH₃, thus imparting a lipid function. In analogs 14, 16 and 18, three of the four Pet unit peripheral carbons were hydroxylated (the remaining carbon being linked to a group comprising the disaccharide moiety). In Aguilera compounds 13, 15 and 17, two peripheral Pet carbons were hydroxylated and the third was sulfated. However, these compounds were found to be inactive as antimitotic agents in all of the cell types, thus discouraging further use of negatively charged groups in analogs of this family.

### SUMMARY OF THE INVENTION

The present invention is directed to non-naturally occurring, biologically active glycosylceramide analogues, and their diagnostic and therapeutic use.

They are preferably immunomodulatory compounds, e.g., ligands for activating Vα14 NKT cells, or to stimulate immune cells to produce specific cytokines. As immunostimulatory compounds, they are useful in enhancing innate immunity, or in adjuvanting the specific immune response to a specific immunogen. They thus may be used to protect a mammal (including a human) against a viral infection, a microbial infection, a parasite or a cancer.

They may alternatively or additionally be immunoinhibitory compounds, in which case they are useful in protection against immune-mediated inflammation and against autoimmune disease. (It should be noted that a compound which promotes a Th1 response and inhibits a Th2 response could be considered to be both immunostimulatory and immunoinhibitory.)

The compounds of the present invention preferably have a molecular weight of less than 10,000 daltons, more preferably less than 5,000 daltons, still more preferably less than 2,500 daltons, even more preferably less than 1,000 daltons.

Broadly speaking, the compounds of the present invention are biologically active (preferably immunomodulatory) compounds which differ from galactosylceramide or another naturally occurring glycosylceramide, at least in terms of the modification or replacement of the ceramide structure, and preferably either the R′ group or the R″ group. Optionally, further modifications may be made: for example, the sugar may be replaced with a different carbohydrate moiety, or even with a pentaerythritol (Pet) unit as hereafter defined. In general, they retain the ceramide nitrogen, at least one lipophilic group attached to the ceramide nitrogen, and a sugar unit or sugar equivalent (the Pet unit).

Thus, in one major aspect the invention relates to non-naturally occurring, biologically active compounds having the formula F-A 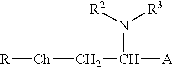
where (italicized terms are formally defined in the Detailed Description below):

R is an organic moiety comprising at least one carbohydrate moiety and/or at least one Pet (pentaerythritol) unit;

Ch is chalcogen (O or S);

R2 is hydrogen, or an organic moiety consisting of at least one primarily alkyl moiety and, optionally, one or more spacers (in any order);

R3 is —C(═Ch)-R3′, where R3′ is an organic moiety comprising a steroid moiety, a terpenoid moiety, an alkaloid moiety, a polyunsaturated moiety or a primarily alkyl moiety, and

A is an organic moiety consisting of at least one primarily alkyl moiety and, optionally, one or more spacers; and

at least one of the following conditions applies:

(1) said compound comprises at least one steroid moiety, and/or at least one alkaloid moiety;

(2) R3′ comprises at least one polyunsaturated moiety (cp. compounds 4-5 in FIG. 11);

(3) R3′ is of the form -(linker)(-spacer-T^{a})ₐ(-T^{b})_{b}, where linker is an aliphatic moiety with not more than 12 non-hydrogen atoms, and consisting of one or more alkyl moieties (which may be substituted with halogen, hydroxyl or sulfhydryl) and/or one or more spacers, a and b are integers each in the range of 0-3, except that a+b is 1 to 3 and, if a=0, b is at least 2, and Ta and Tb are, independently, organic moieties consisting of at least one primarily alkyl moiety and, optionally, one or more spacers;

(4) A is —CH(-spacer-R4)-R1 where

(A) R1 is hydrogen, and R4 is hydrogen or an organic moiety consisting of at least one primarily alkyl moiety and, optionally, one or more spacers;

(B) R1 is an organic moiety consisting of at least one primarily alkyl moiety and, optionally, one or more spacers (in any order), and R4 is an organic moiety consisting of at least one primarily alkyl moiety and, optionally, one or more spacers;

(C) R1 is -(spacer cluster)-(organic moiety) and R4 is hydrogen, -(organic moiety), or -(spacer)-(organic moiety), where each organic moiety is one consisting of at least one primarily alkyl moiety and, optionally, one or more spacers;

(5) A is -(spacer cluster)-R1, where R1 is hydrogen or an organic moiety consisting of at least one primarily alkyl moiety and, optionally, one or more spacers.

Note that one, two, three or four of conditions (1)-(5) may apply, except that (4) and (5) are mutually exclusive.

Whenever in this specification we recite “organic moiety consisting of at least one primarily alkyl moiety and, optionally, one or more spacers”, it is to be understood that these components can occur in any order.

Preferably, each of the organic moieties referred to above consists of not more than 120 atoms other than hydrogen atoms.

The carbohydrate moiety is preferably a monosaccharide. Each sugar unit in the carbohydrate moiety is preferably a pentose, or hexose, or nonose. Galactose is especially preferred, and alpha-Galactose is most preferred.

R may comprise, besides the carbohydrate moiety, one or more phosphate equivalents. Preferably, these are sugar unit substitutents.

Whenever this disclosure to refers to use of chalcogen, it will be understood that oxygen is the preferred embodiment thereof.

A primarily alkyl moiety may be a polyunsaturated moiety, and vice versa.

R2 is preferably hydrogen.

R3 preferably comprises at least one strongly lipophilic group. More preferably R3 is a strongly lipophilic group.

A preferably comprises at least one strongly lipophilic group. More preferably A is a strongly lipophilic group.

Condition (1) introduces a steroid or alkaloid moiety anywhere into the ceramide structure. Preferably, it is incorporated into R3′, which corresponds to the hydrophobic (“fatty”) portion of the normal fatty acyl moiety of the natural glycosylceramides. A steroid moiety is preferred.

Condition (2) introduces a polyunsaturated moiety into R3′. Preferably, it comprises at least one methylene-interrupted pair of alkenic double bonds (—C═C—C—C═C—). More preferably, all double bonds in the moiety are methylene interrupted. Preferably there are 3-6 double bonds, more preferably four. A PUM with four double bonds, with each adjacent pair methylene interrupted, is especially preferred. It is most preferred that R3 have the arachidonic acid carbon skeleton, —C(═O)—C—C—C—C═C—C—C═C—C—C═C—C—C═C—C—C—C—C—C.

Condition (3) also modifies the fatty acyl moiety of the normal glycosylceramide. It introduces a linker moiety between the carbonyl carbon (C═O or C═S) and each moiety T^{a} and/or T^{b}, the latter more or less corresponding to the fatty portion of the normal fatty acyl moiety. This portion may be a divalent (a+b=1), trivalent (a+b=2) or tetravalent (a+b=3) moiety. In the latter two cases, the normal fatty acyl moiety, which is linear, is effectively replaced by a two- or three-branched structure.

It will be appreciated that the number of moieties T^{a} will be equal to the value of a, and the number of moieties T^{b} will be equal to the value of b. If there is more than one T^{a}, they may be the same or different. Likewise, if there is more than one T^{b}, they may be the same or different. Naturally, each T^{a} may be the same as or different from a given T^{b}, and vice versa.

Preferably each T^{a} and each T^{b} is a primarily alkyl moiety. The principal distinction between them is that each T^{a} moiety is linked to the remainder of the compound by a spacer, and each T^{b} moiety is linked directly, i.e., by a C—C bond. Preferably, b=0, i.e., the linker is connected to the primarily alkyl moieties by spacers.

The linker may, but preferably does not, include halogen, hydroxyl or sulfhydryl groups.

When the linker is a divalent moiety, R3′ is preferably of the form —CH2-(spacer)-*, where * denotes the linked primarily alkyl moiety. The preferred spacers are —C(═O)— and —O—.

When the linker is a trivalent or tetravalent moiety, branching will usually occur at a carbon atom of the linker, but may also occur at a nitrogen atom. R3′ is preferably of the form —CH2-CH(-R3′Rem2)-R3′Rem1, and R3′Rem1 and R3′Rem2 are independently chosen organic moieties consisting of at least one primarily alkyl moiety and, optionally, one or more spacers.

More preferably R3′ is of one of the following forms:

—CH2-CH(-*)-(spacerA1)-(spacerA2)-*

—CH2-CH(-*)-(spacerA)-*

—CH2-CH(-(spacerB)-*)-(spacerA1)-(spacerA2)-*

—CH2-CH(-(spacerB)-*)-(spacerA)-*

—CH(-*)-(spacerA1)-(spacerA2)-*

—CH(-*)-(spacerA)-*

—CH(-(spacerB)-*)-(spacerA1)-(spacerA2)-*

—CH(-(spacerB)-*)-(spacerA)-*

where each * denotes a linked primarily alkyl moiety (these may be the same or different), SpacerA1 is preferably —NH— or —O—, Spacer A2 is preferably —C(═O)—, SpacerA is preferably —O—, and SpacerB is preferably —O—.

The linker may comprise a spacer cluster, or, in conjunction with spacerA, spacerA1, spacerA2 or spacerB, it may form a spacer cluster.

While this embodiment of R3′ could be referred to as a two branched moiety, because of the two-way branching provided by the linker, it will be understood that either or both of the linked primarily alkyl moieties may be branched itself, so that R3″ effectively has more than two branches.

Finally, the linker may be tetravalent, serving to link three primarily alkyl moieties to the remainder of the molecule (by the route N-spacer-linker).

Preferably, at least one of the linked primarily alkyl moieties is substantially linear, more preferably linear. Preferably, both are.

Preferably, at least one of the linked primarily alkyl moieties is strongly lipophilic.

Condition (4) modifies the portion of the sphingoid base which is distal to the sugar in the normal glycosylceramide. This portion is normally —CH(—OH)-alkyl. As a result of the operation of condition (4), various modifications can occur: (a) the alkyl is replaced by hydrogen, (b) the hydroxyl is replaced by a spacer-linked moiety which is not hydrogen, or (c) the alkyl is replaced by a spacer cluster-linked organic moiety.

In condition (4)(a), preferably R4 is hydrogen,—(primarily alkyl), or -(spacer)-(primarily alkyl). In condition (4)(b), preferably R1 and R4 are independently—(primarily alkyl), or -(spacer)-(primarily alkyl). In condition (4)(c), the cited organic moieties of R1 and R4 are preferably both primarily alkyl moieties (the same or different).

Condition (5) sets out yet another variation in terms of modification of the distal portion of the sphingoid base. Here, the interesting feature is the spacer cluster. Preferably, the organic moiety within the group A as defined by (5) is a primarily alkyl moiety. More preferably, it is strongly lipophilic.

When (4) or (5) apply, and R1 is primarily alkyl, R1 is preferably primarily alkanyl, or a primarily alkyl moiety with a single C═C bond and no triple bonds. In the latter case, the C═C bond is preferably between C-2 and C-3 (carbons numbered from the first carbon of R1, the one nearest the sphingoid nitrogen), as in compound 5 of FIG. 11.

In a second major aspect, the compounds of the present invention may be of the form R—O-Z, where R is an organic moiety comprising a carbohydrate moiety, and Z is an organic moiety comprising a steroidal, terpenoidal or alkaloidal moiety (cp. compounds 8-11 in FIG. 12). Such compounds may, but need not, also belong to formula F-A of the first major aspect.

The preferences for R are the same as for the compounds of the first major aspect.

Preferably Z consists of said steroidal, terpenoidal or alkaloidal moiety, and, optionally, one or more primarily alkyl moieties and/or one or more spacers. Z preferably comprises a steroidal moiety. Preferably, Z comprises not more than one spacer or spacer cluster, and not more than one primarily alkyl moiety (not counting any portion of said steroidal, terpenoidal or alkaloidal moiety as part of said primarily alkyl moiety). Preferably Z consists essentially of said steroidal, terpenoidal or alkaloidal moiety.

In a third major aspect, the compounds of the present invention may comprise a Pet unit. If so, they are of one of the following forms:

(1) one arm of the Pet unit is connected to the O-1 atom of a ceramide and the other arms are connected to hydrogen or an organic moiety; or

(2) one arm of the Pet unit is a —CH2-NH— arm and is connected to an organic moiety consisting of at least one primarily alkyl moiety and optionally one or more spacers, a second arm is a —CH2-Ch- arm and is connected to an organic moiety consisting of at least one primarily alkyl moiety and optionally one or more spacers, and the remaining arms are connected to hydrogen, or an organic moiety,

with the caveat that the compound does not comprise a phosphate equivalent.

The aforementioned caveat is imposed to avoid overlap with the disclosure of lipid A analogues, based on the Pet unit, in our PCT/US03/14633 filed 9 May 2003, hereby incorporated by reference in its entirety.

Preferably, the compounds of the present invention are not identical to any compound disclosed or claimed in the above-identified application.

In case (1) the Pet unit replaces at least one sugar unit of a normal glycosylceramide. In case (2), the Pet unit replaces a portion of the sphingoid base moiety of a normal glycosylceramide.

The organic moiety is preferably not more than 120 atoms other than hydrogen. The organic moiety is preferably an organic moiety comprising a carbohydrate moiety, an organic moiety comprising another Pet unit, an organic moiety comprising a polyunsaturated moiety, a steroid moiety, a terpenoid moiety and/or an alkaloid moiety, or an organic moiety which is primarily alkyl.

Such compounds may, but need not, also belong to formula F-A.

In a fourth major aspect, the compounds of the present invention are fluorinated glycosylceramide analogues, defined by the general formula F-AF: 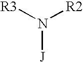
where R2 is hydrogen or an organic moiety; J is an organic moiety comprising at least one sugar unit and/or at least one Pet (pentaerythritol) unit; R3 is of the form -(Z)₀₋₁-CF2-R3′, Z is a single spacer, -spacer-CH2-spacer-, or a spacer cluster, and R3′ is a primarily alkyl moiety.

Preferably, there is one Z, and more preferably, it is a single spacer, most preferably —C(═O)—.

Preferably R3″ is strictly alkyl. It should be noted that under the definition of “primarily alkyl”, any, some or all of the carbon atoms of R3′ (and R3″) can be fluorinated, too.

Note that in these compounds, a terminal primarily alkyl moiety is fluorinated, and such fluorination includes the carbon of that moiety which is closest to the sphingoid nitrogen, whereas in the compounds of Faroux-Corlay, only the distal carbons of the terminal primarily alkyl moiety are fluorinated.

In general, for all compounds of the present invention, a moiety that is “primarily alkyl” is preferably also substantially linear and/or strongly lipophilic.

Preferably, at least one (and more desirably both) of the A and R3 groups of the various formulae is a group which has at least 5, more preferably at least 10, even more preferably at least 15, still more preferably at least 20, carbon atoms. In this regard, note that the R3 group corresponds roughly to the fatty acyl group of the natural glycosylceramide, and the A group to a portion of the sphingoid base, i.e., to C-3 and beyond. Hence, the preferences discussed in the “ceramide replacement” section below apply, mutatis mutandis, as preferences for R3 and A.

Preferably, each of the R1, R2 , R3, R and A groups of the various formulae is a group with not more than 40, more preferably not more than 30, carbon atoms.

Any moiety identified as a linker moiety is preferably not more than ten atoms other than hydrogen.

### BRIEF DESCRIPTION OF THE DRAWINGS

FIG. 1 shows structures of a natural α-GalCer, AGL-9b, which was isolated from marine sponge and exhibited potent anti-tumor activity; and a synthetic analogue, KRN7000, which is currently being evaluated as a therapeutic agent in clinic.

FIG. 2 shows various structures that can be incorporated into ceramides in the design of α-GalCer analogues. Unsaturated fatty acids and fluoro-substituted lipids can modulate the flexibility of the lipid chains, which in turn affect the antigen presentation of these α-GalCer derivatives by CD1d molecules to T-cell receptors and thus modulate their biological activities. Similarly, di-lipo-fatty acid and serine-containing fatty acid all contribute to the lipophilic nature of α-GalCer.

FIG. 3 shows α-GalCer analogues containing unusual N-acyl groups on natural sphingosine.

FIG. 4 shows α-GalCer analogues having unnatural N-acyl groups on sphingosine which carries a E-4,5-double bond. The E-4,5-ene-sphingosine has not been found for natural α-GalCer molecules from marine sponge, but is present in gangliosides from mammalian sources.

FIG. 5 shows α-GalCer analogues where the galactose is replaced by GalNAc and the ceramide carries an unusual N-acyl group.

FIG. 6 shows α-GalCer analogues wherein the core of sphingosine base is substituted by a structural mimic serinol.

FIG. 7 shows α-GalCer analogues wherein the core of sphingosine base is substituted by a simple serine. The carboxylic group of serine can be esterified, amidated, or exist as free acid form. Two of these structures contain two units of L-serine.

FIG. 8 shows α-GalCer analogues containing chemically modified sphigosine in that the carbon chain is disrupted by incorporating heteroatoms, e.g., O, NH and S, in the form of ether, ester, or amide linkages.

FIG. 9 shows α-GalCer mimics containing an amino-substituted pentaerythritol unit to mimic the core of natural sphingosine base. The remaining unsubstituted hydroxyl group of pentaerythritol in these structures represents the free 3-OH group of natural sphingosine which is essential for the manifestation of biological activities of α-GalCer derivatives.

FIG. 10 shows examples of α-GalCer analogues having two galactose units built on a pentaerythritol molecule. These structures are designed as divalent antigens in which two galactose units may be recognized by dimerized receptors.

FIG. 11 shows the structures of α-GalCer analogues (1-7) which have been prepared as examples of the present invention. Structure 1-4 is based on serinol as structural mimic of the core of sphingosine base, and structures 4 and 5 incorporate an arachidonic acid moiety. Structure 7 is identical to KRN7000 (FIG. 1) while the sphingosine in structure 5 and 6 contains a double bond which is common in the sphingoid bases of natural beta galactosyl ceramides, but very rarely in sphingoid bases of natural alpha galactosyl ceramides. However, to the best of our knowledge, structures 5 and 6 per se do not occur in nature and have not previously been synthesized.

FIG. 12 shows structures of steroidal galactopyranosides (8-13) derived from plant-originated sterols as potential functional mimics of α-GalCers. Both α- and β-glycosides are prepared for biological evaluation.

FIG. 13 shows the synthetic pathway for α-GalCer analogues (1-3). The known galactosyl fluoride 14 is employed to construct the desired α-glycosidic linkage. Protecting group manipulation led to the formation of amino-derivative 18, which was coupled to fatty acid moieties (19-21) to give 22-24. Final deprotection provided the designed products 1-3.

FIG. 14 shows the preparation of α-GalCer analogue 4. A new galactosyl donor 29 was prepared. Glycosylation reaction between the donor 29 and the acceptor 30 provided the α-linked galactooside 31 in good yield. Standard protecting group manipulation and final introduction of arachidonic acid (35) afforded the designed α-GalCer analogue 4.

FIG. 15 shows the preparation of suitably protected sphingosine acceptor 41 from the commercially available sphingosine 37.

FIG. 16 shows the preparation of α-GalCer analogue 5. The method is generally applicable for preparing α-GalCer analogues with double bond(s) in the aglycone moiety.

FIG. 17 shows the synthetic pathway for α-GalCer analogue 6 and 7.

FIG. 18 shows the preparation of steroidal glycoside 8.

FIG. 19 shows the preparation of steroidal glycoside 9.

FIG. 20 shows the preparation of steroidal glycoside 10.

FIG. 21 shows the preparation of steroidal glycoside 11.

FIG. 22 shows the preparation of steroidal glycoside 57α and 57b.

FIG. 23 shows the preparation of steroidal glycoside 12 and 13.

FIG. 24 show cytokine secretion by BALB/c Spleen cells, as determined by ELISA. The figure refers to BC1-041, BC1-049, KRN7000 and alphaGalCer (Besra) as “antigens” but immunomodulatory compounds” would be more accurate. In each case, one novel compound is compared with KRN7000 and alphaGalCer (Besra). It is BCI-041 in 24(a) and (b), and BC1-049 in 24(c) and (d). The abscissa shows the antigen concentration in ng/ml.

The ordinate is IFNgamma (ng/ml) in 24(a) and (c), and IL4 (pg/ml) in 24(b) and (d).

FIG. 25 shows

25(a) proliferation of Balb/C WT splenocytes in response to various concentrations of alpha-Gal,Cer -GluCer, -ManCer, and of Veh (vehicle).

25(b) IFN-gamma production (ng/ml) in response to various concentrations of alpha-GalCer, -GluCer, -ManCer,and anti-CD3.

25(c) IL-4 production (pg/ml) in response to various concentrations of alpha-GalCer, -GluCer, -ManCer,and anti-CD3.

25(d) proliferation in response to various concentrations of compounds 038, 040, 041, 049, 050, anti-CD3, or in absence of antigen.

25(e) IFN-gamma production in response to various concentrations of compounds 038, 040, 041, 049, 050, anti-CD3.

25(f) IL-4 production in response to various concentrations of compounds 038, 040, 041, 049, 050, anti-CD3.

25(g) proliferation in response to various concentrations of compounds 033, BF84, 046, 047, 048, anti-CD3, or in absence of antigen.

25(h) IFN-gamma production in response to various concentrations of compounds 033, BF84, 046, 047, 048, anti-CD3

25(i) IL-4 production in response to various concentrations of compounds 033, BF84, 046, 047, 048, anti-CD3.

FIG. 26 shows the effect of various compounds (BC1-041, BC1-049, BC1-050, BF-1508-84 and anti-CD3) on proliferation of Balb/C CD1−/− cells, as a function of “antigen” concentration.

FIG. 27 shows IFN-gamma and IL4 production, as elicited in Balb/C or B6 strains, as a result of OCH, BF1508-84, and KRN-7000. OCH is disclosed by Miyamoto (2001) and has a C24 fatty acyl moiety and a C9 sphingoid moiety, hydroxylated at carbons 3 and 4, and O-linked to galactose at its carbon 1.

FIG. 28 shows proliferation of splenocytes in (a) Balb/C or (b) B6 strains, as a result of OCH, BF1508-84, and KRN-7000.

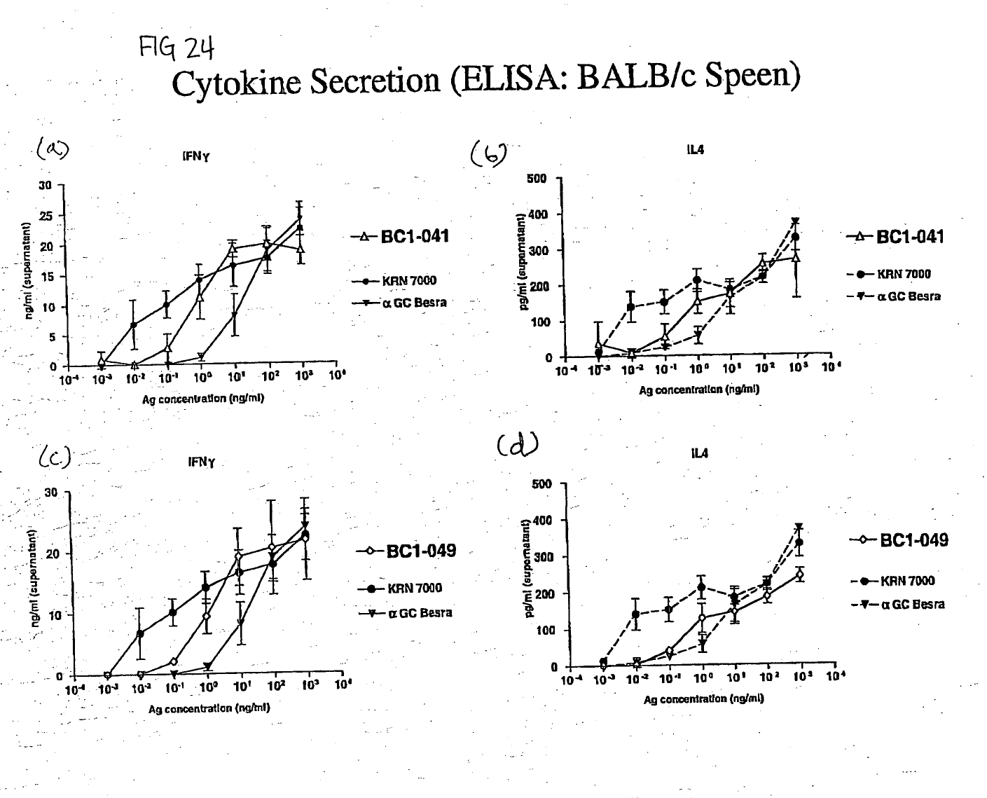

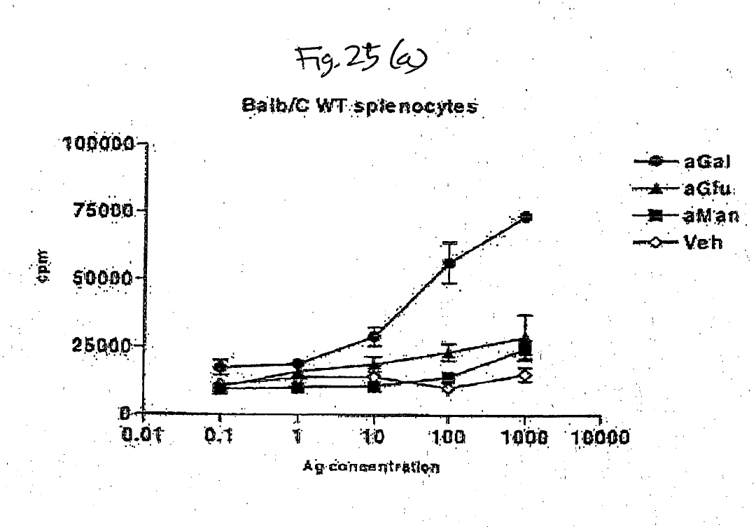

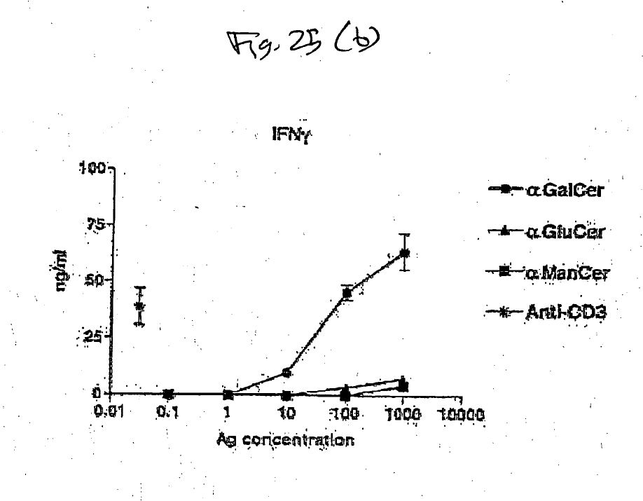

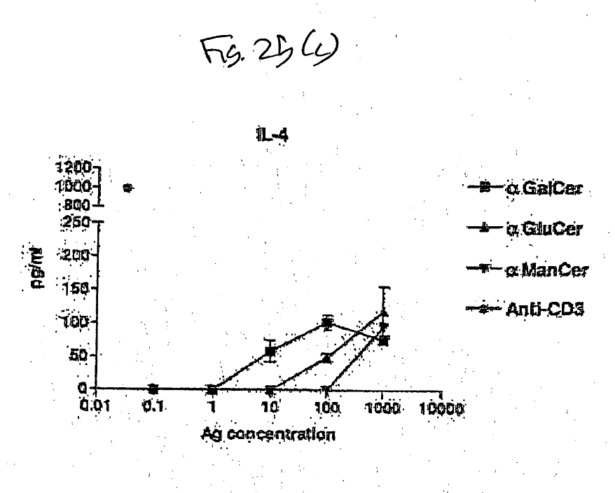

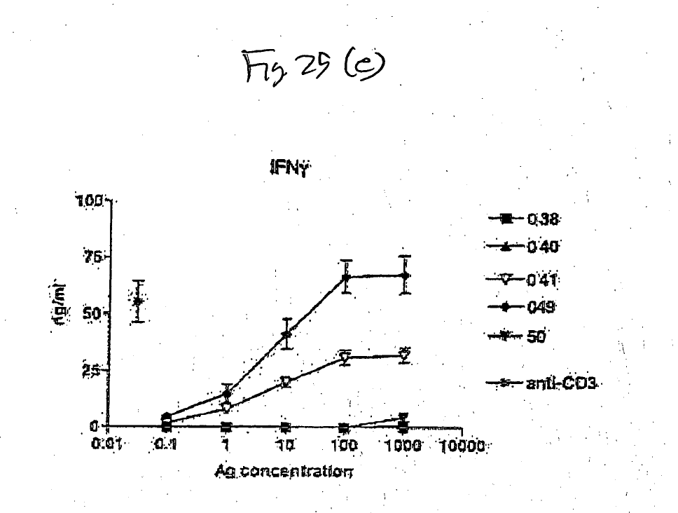

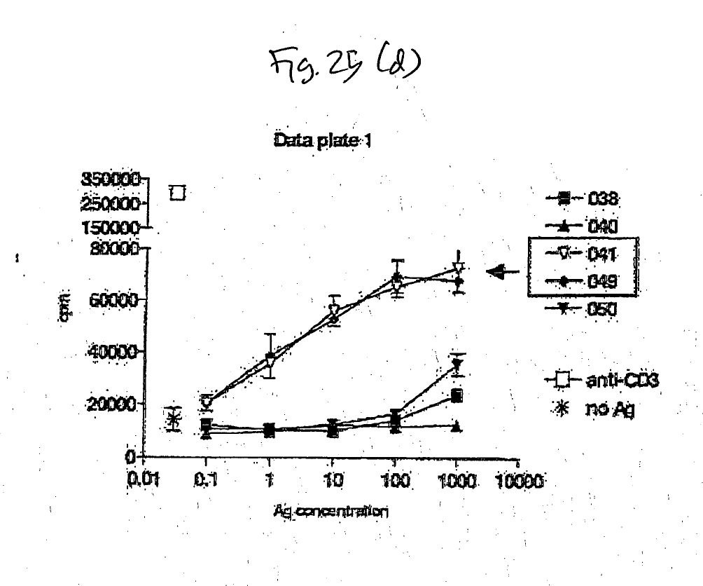

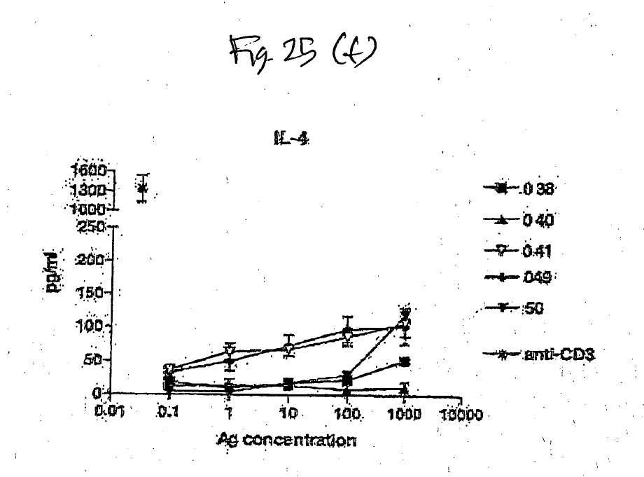

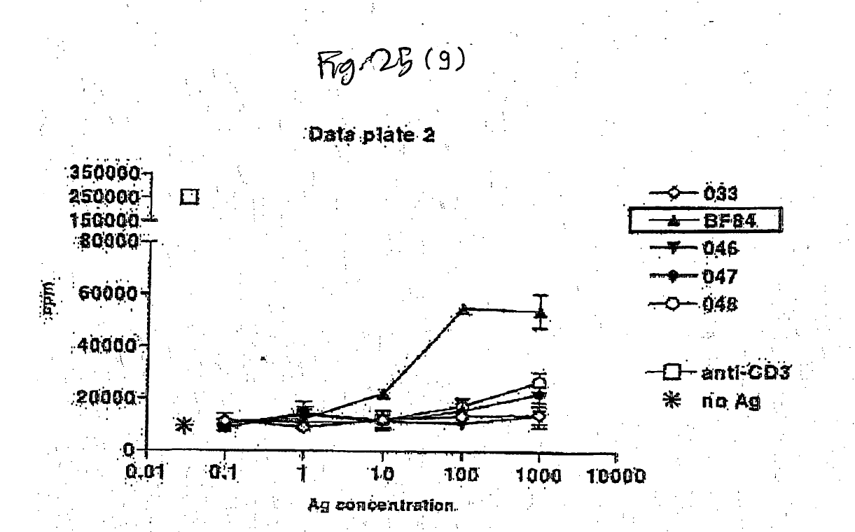

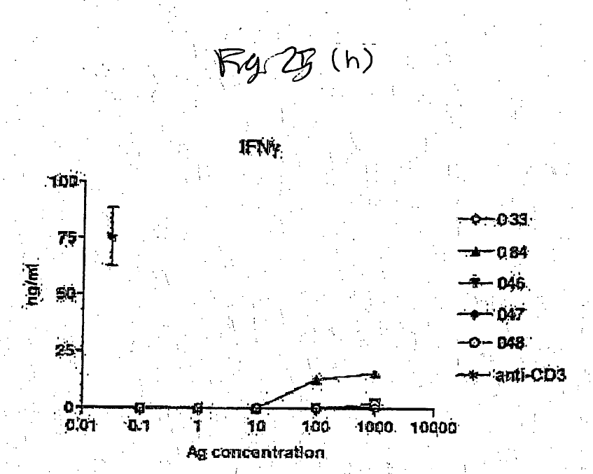

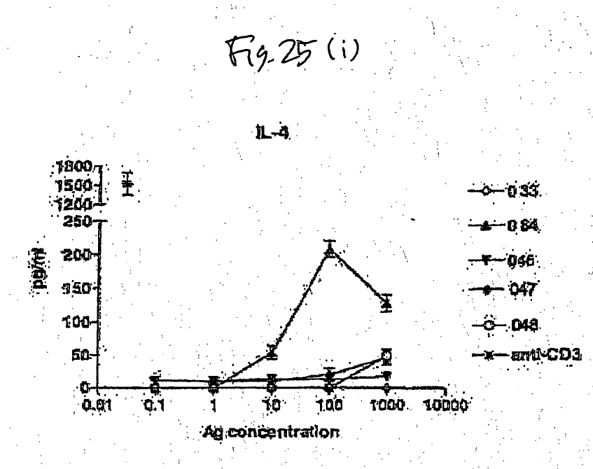

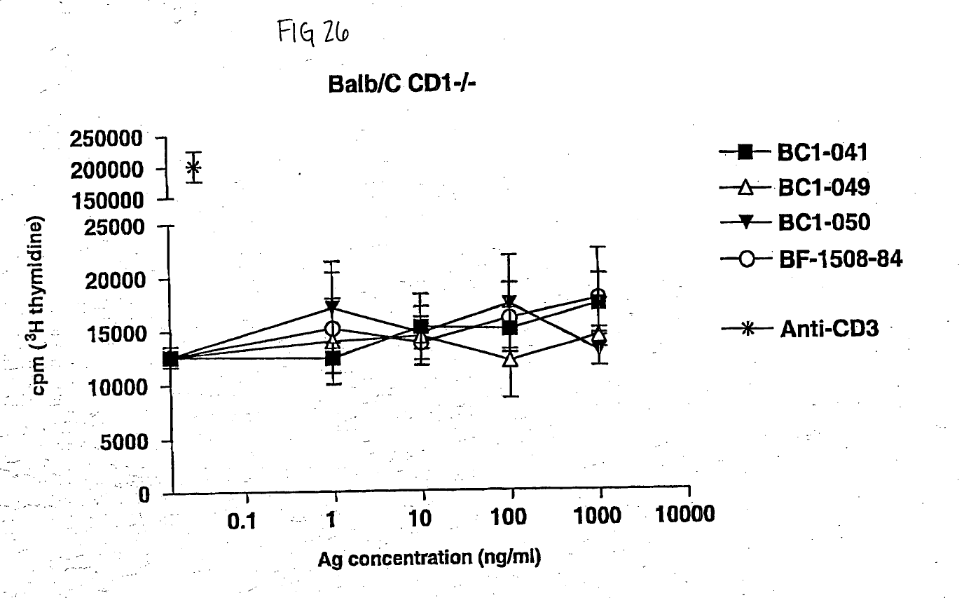

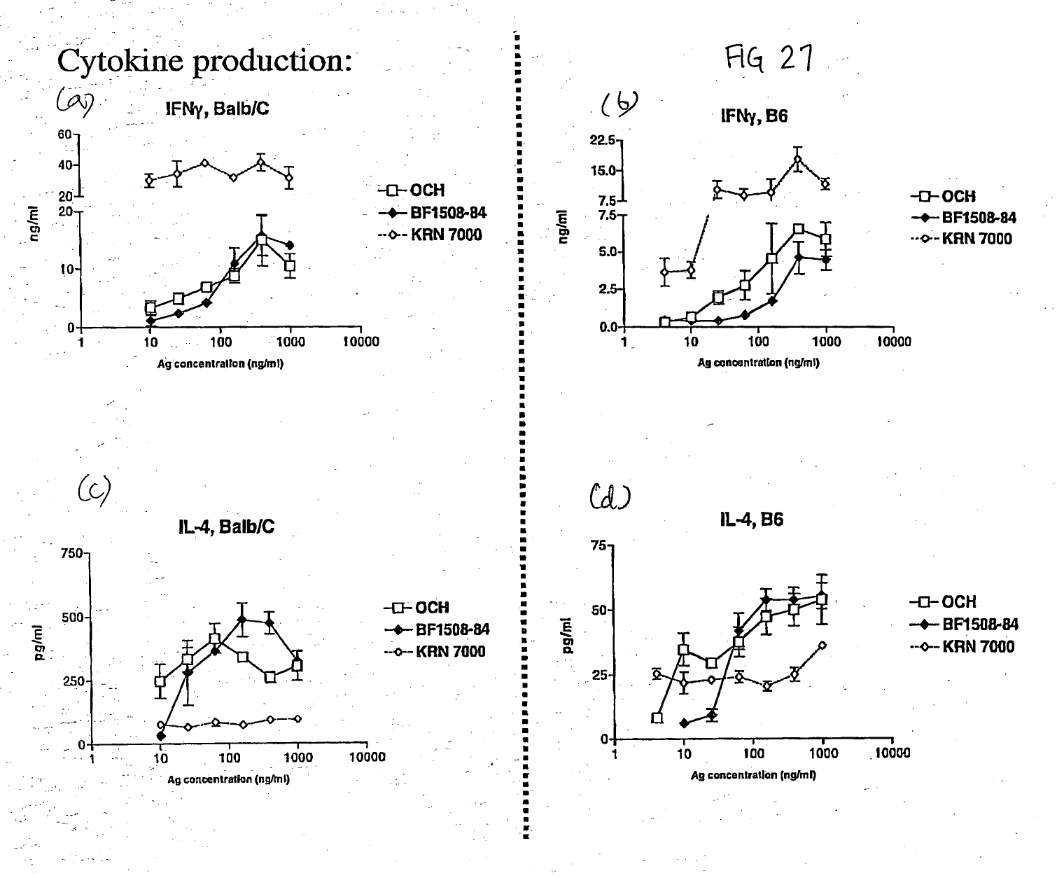

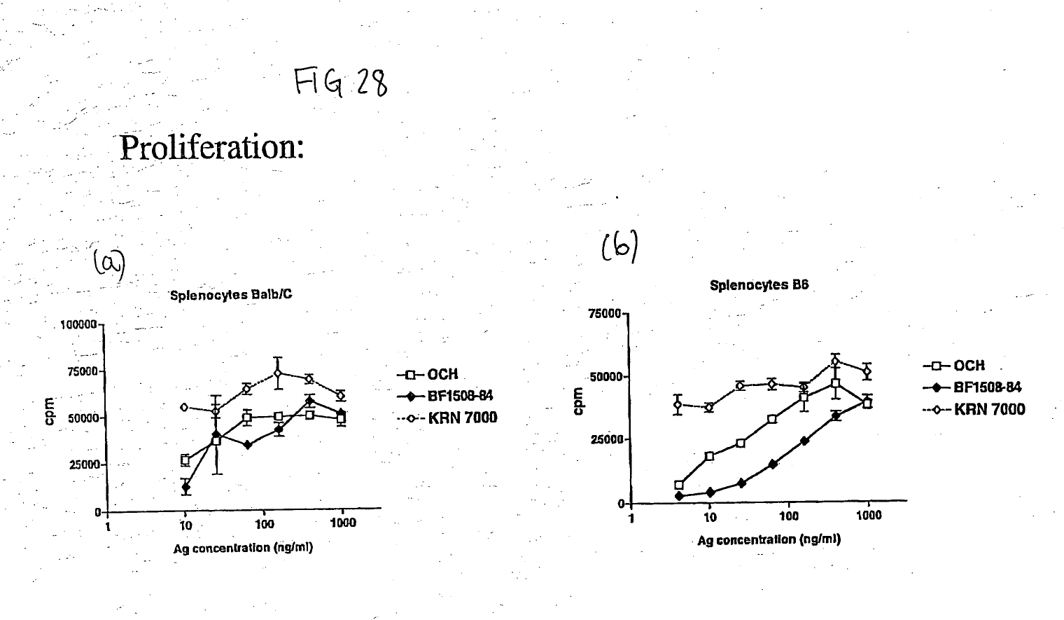

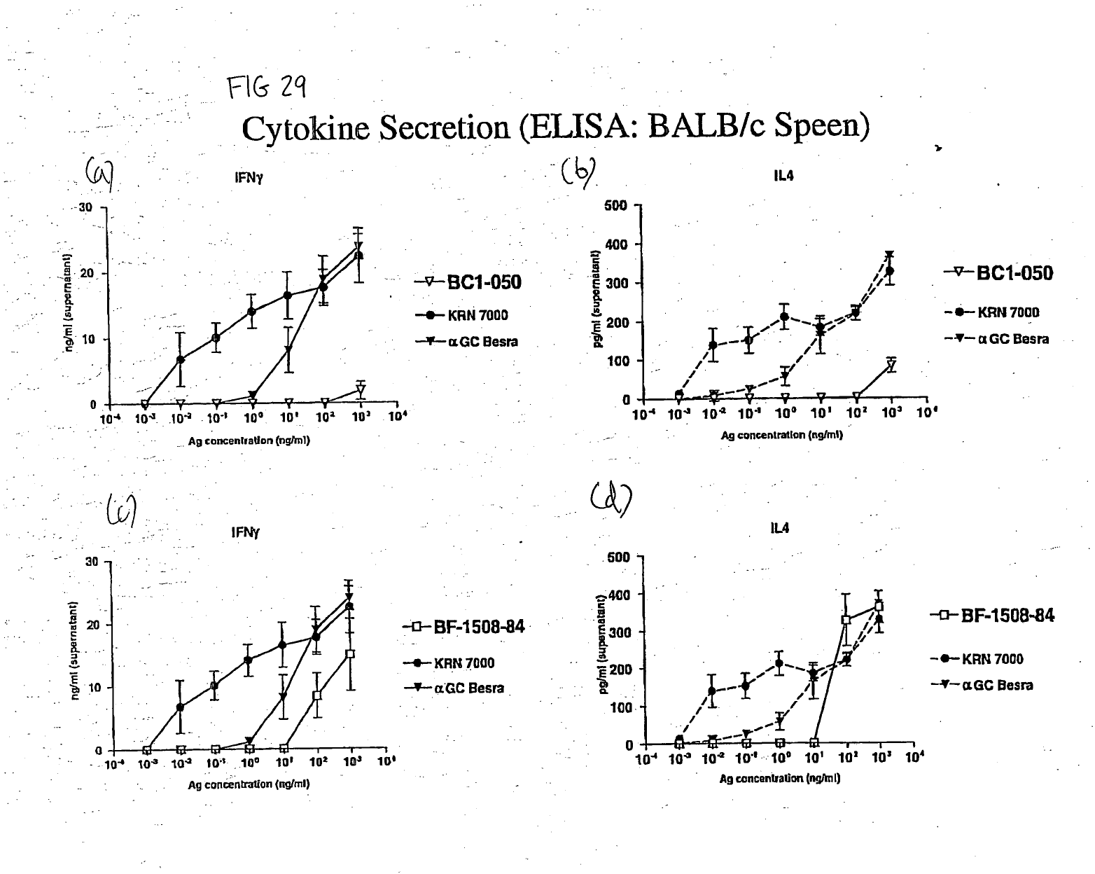

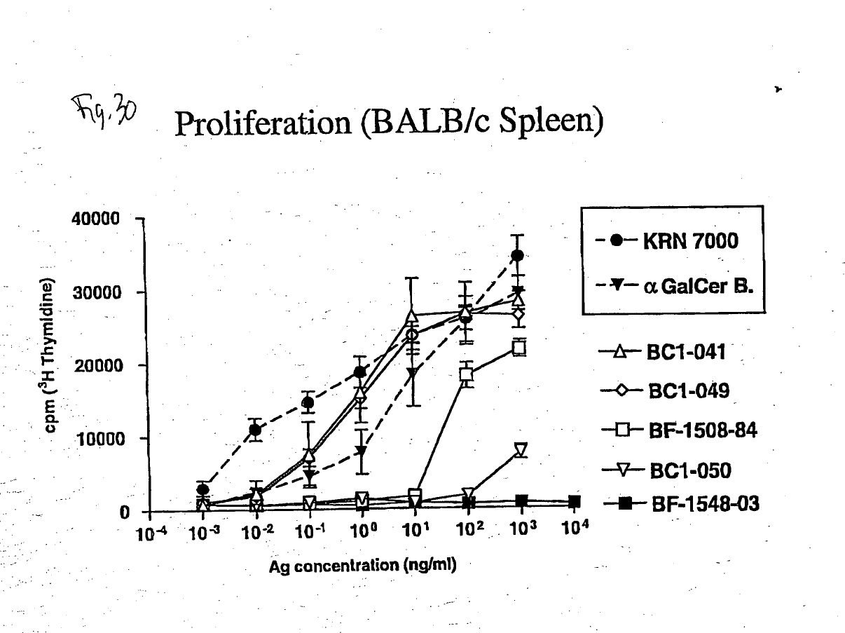

FIG. 29 is similar to FIG. 24, but the compounds shown are BC1-050 in 26(a) and (b), and BF-1508-84 in 24(c) and (d).

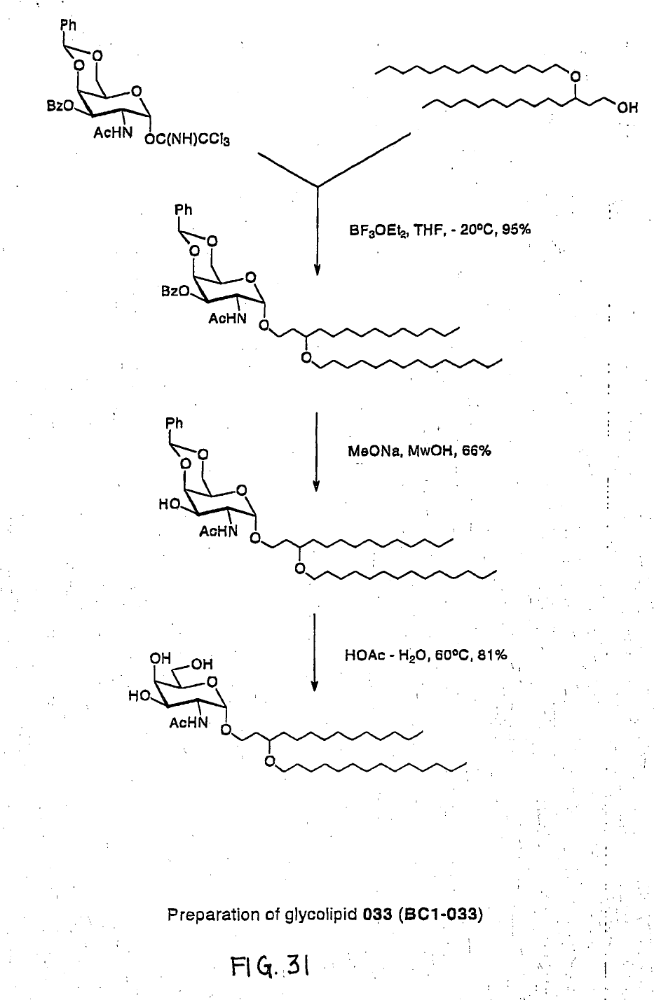

FIG. 30 is similar to FIG. 25, but the compounds are KRN-7000, alpha-Gal Cer, BC1-041, BC1-049, BF-1508-84, BC1-050 and BF-1548-03.

FIG. 31 shows the preparation of glycolipid 033 (BC1-033).

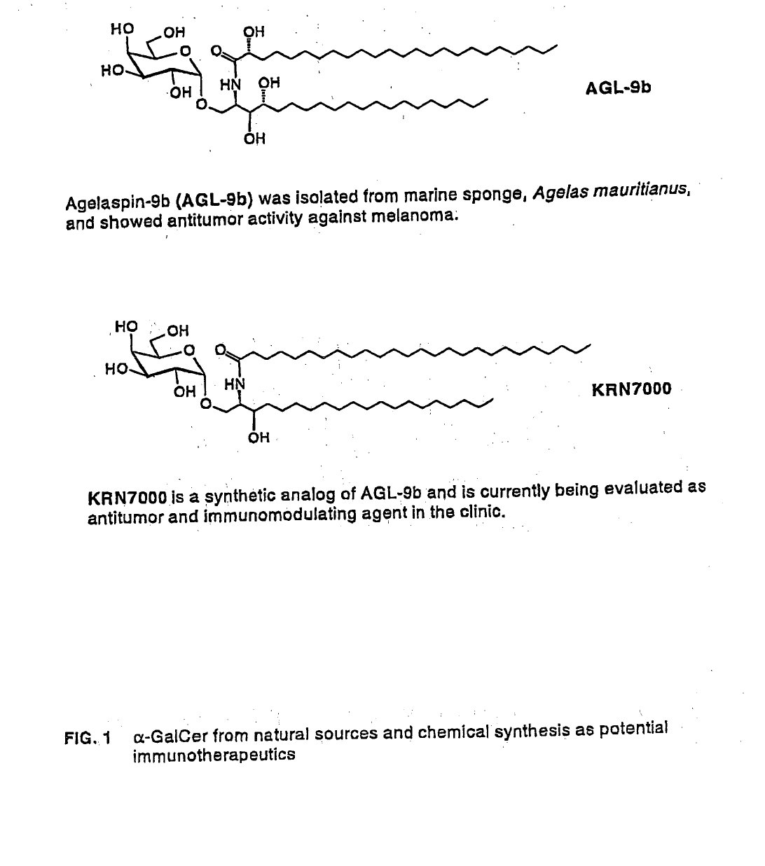

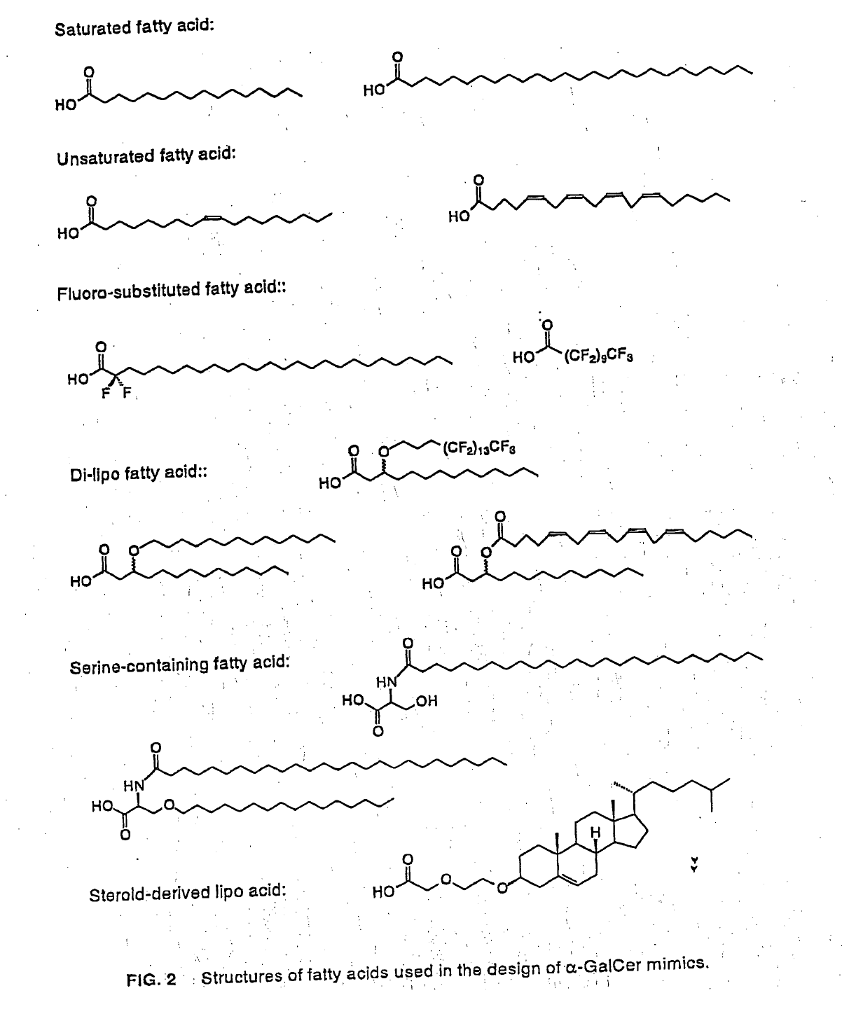

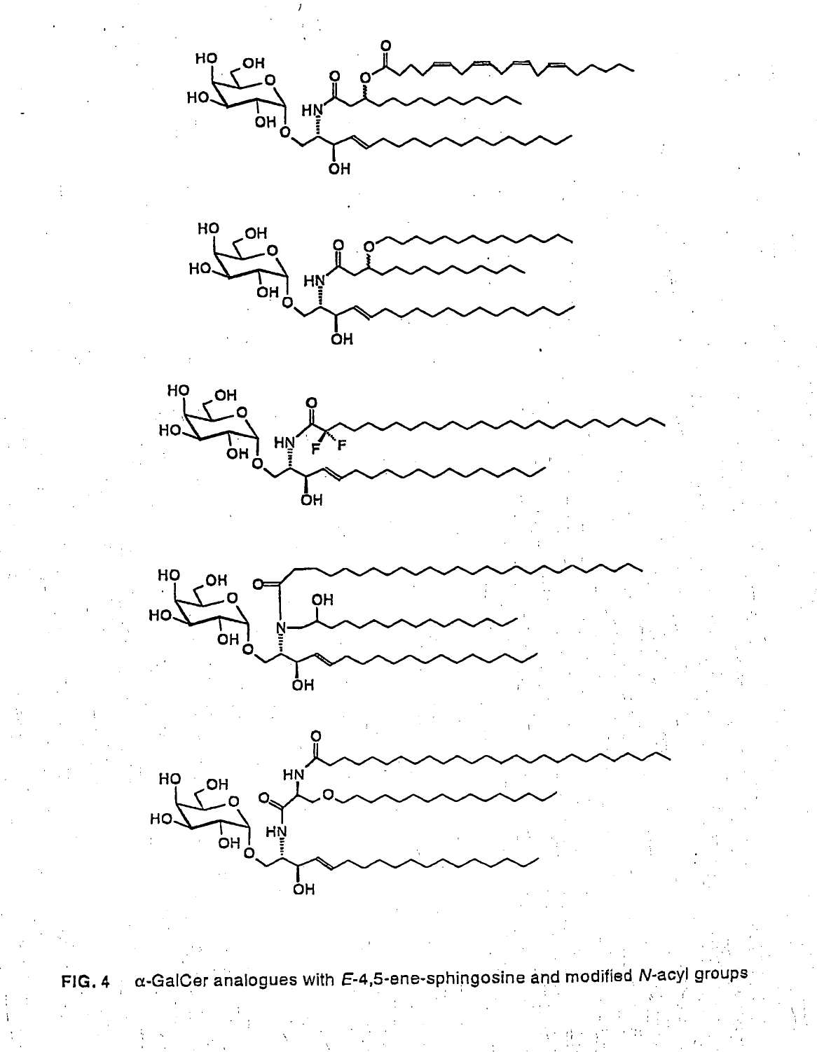

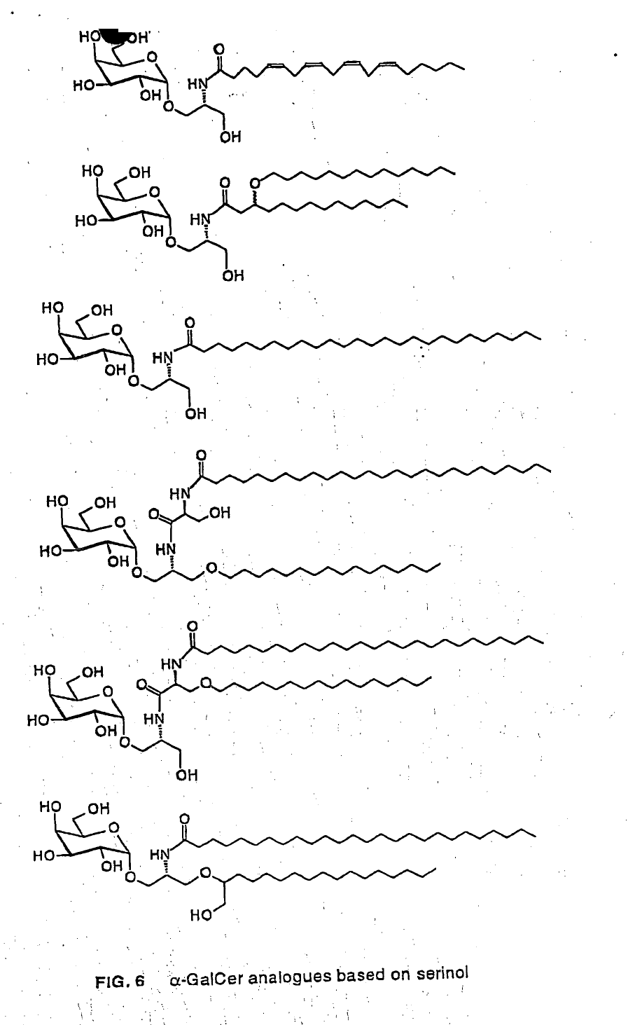

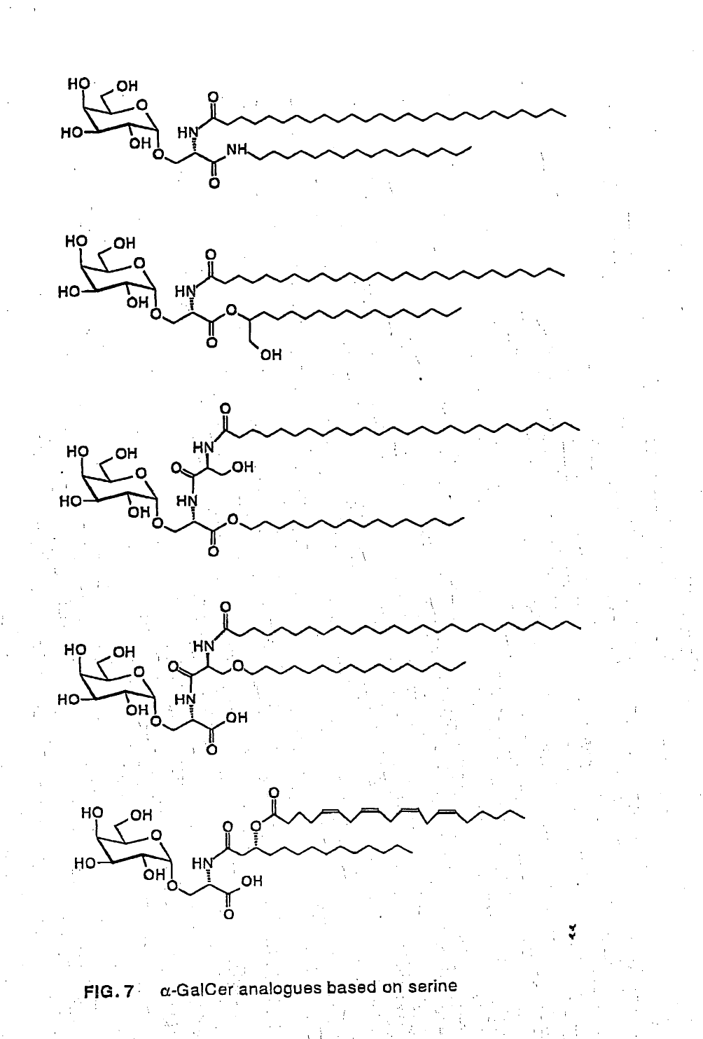

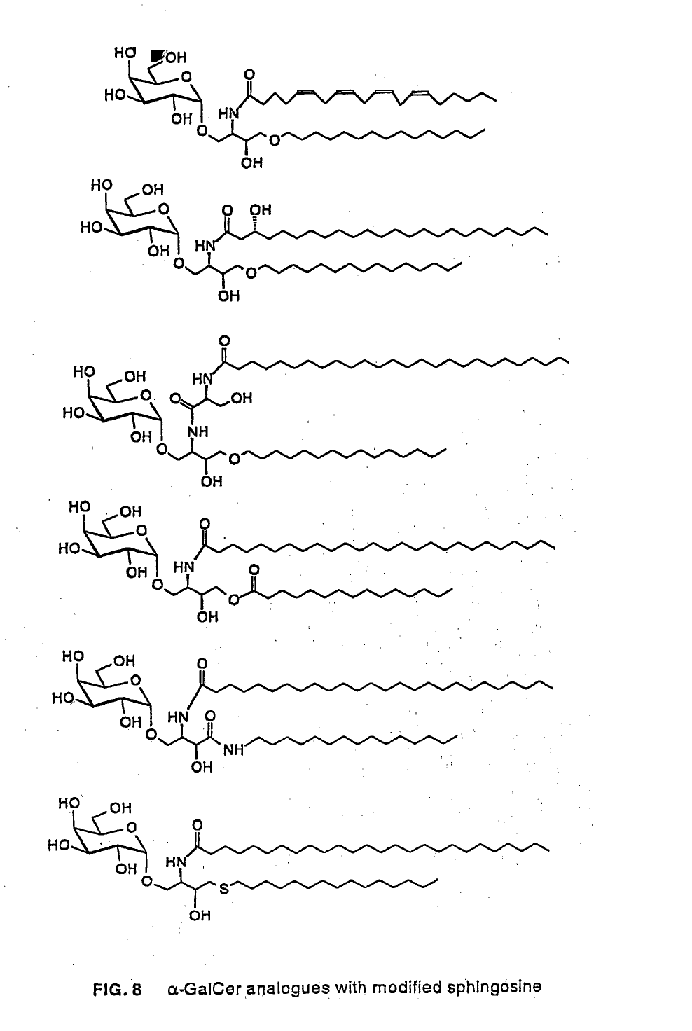

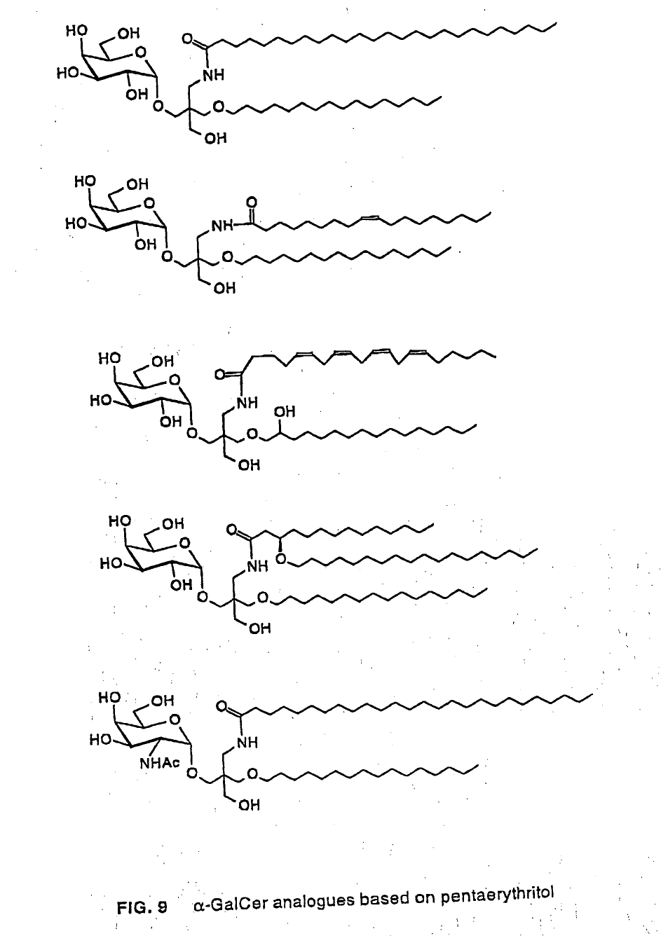

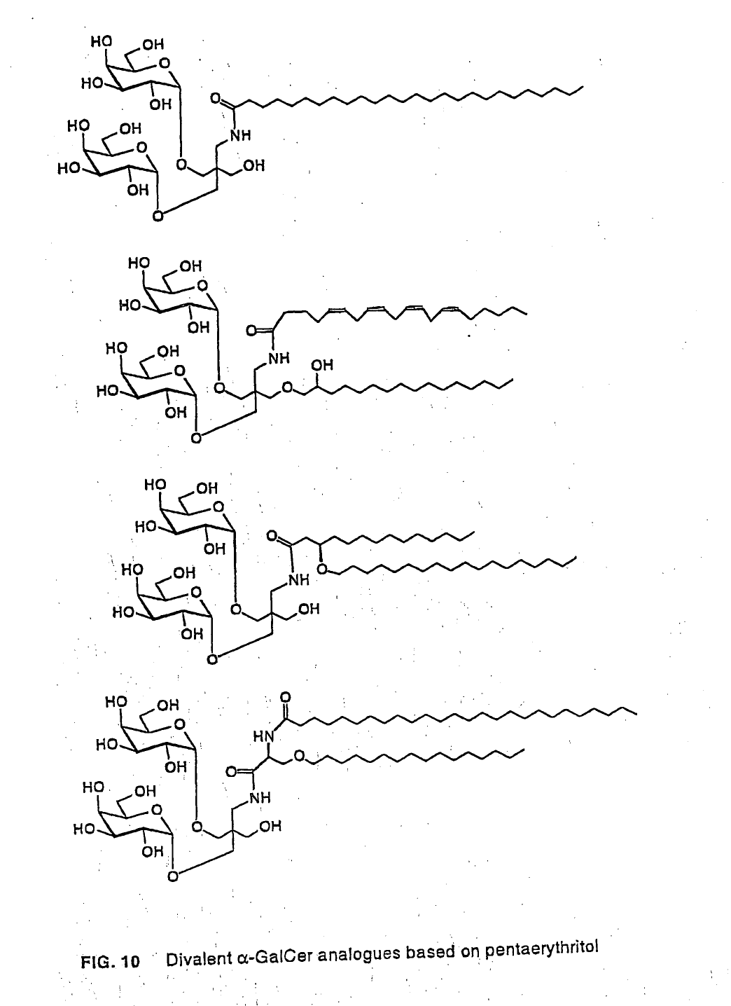

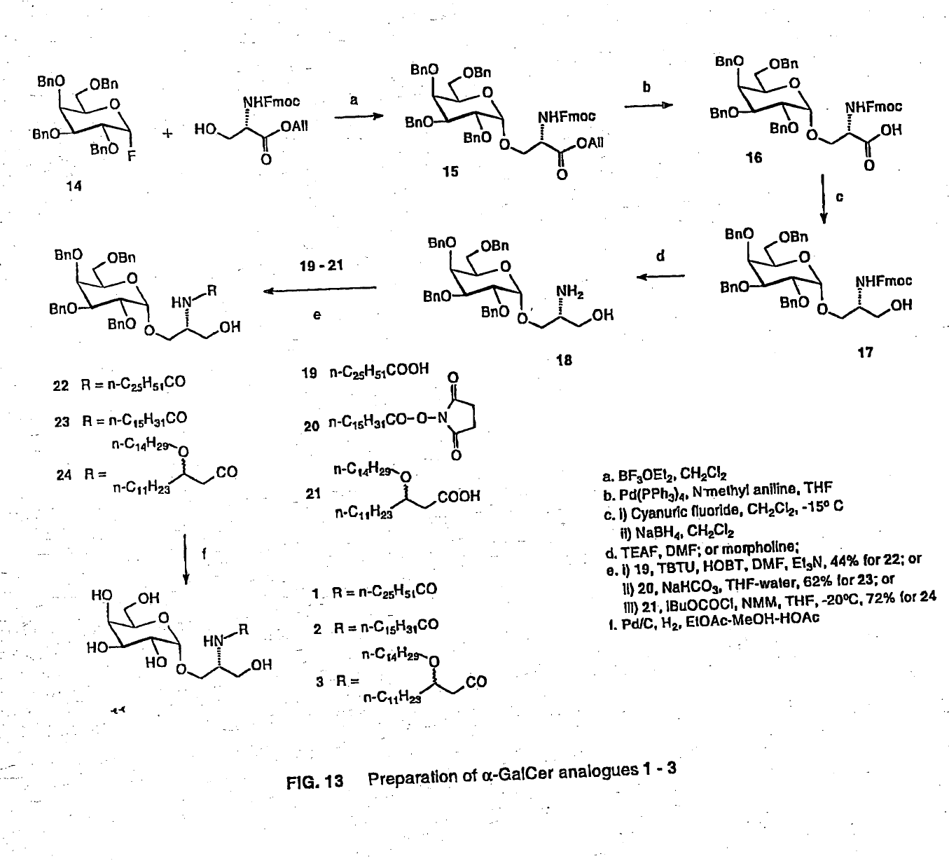

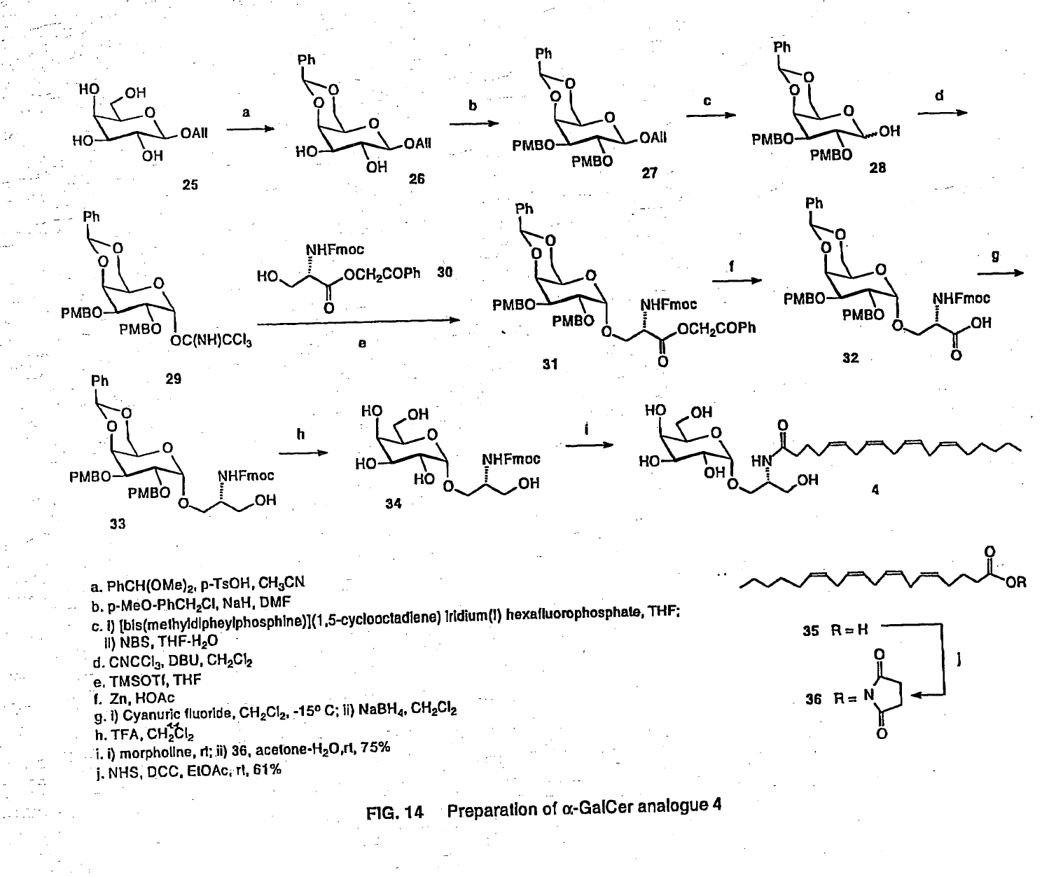

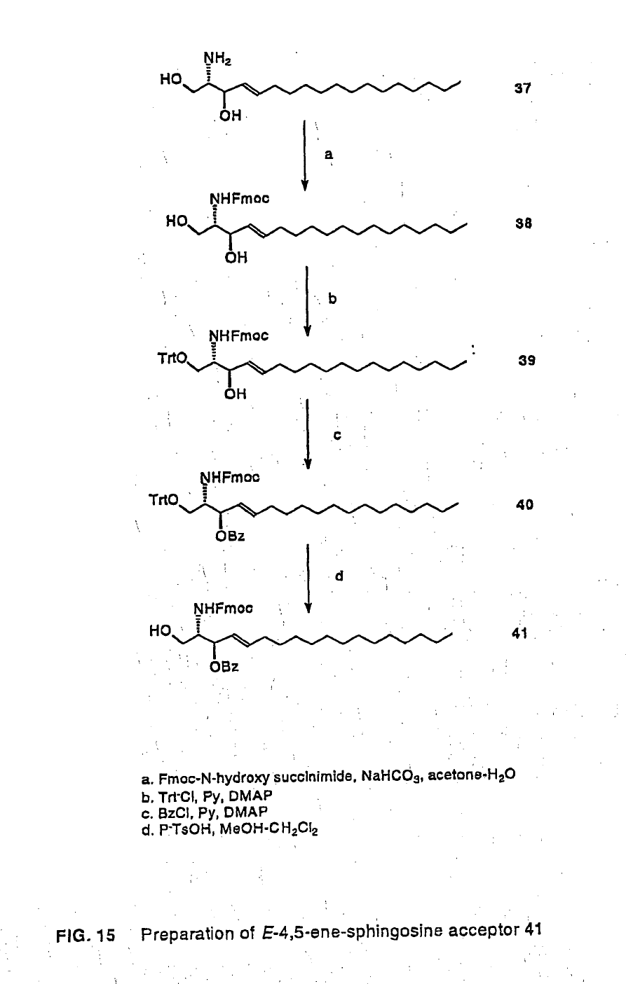

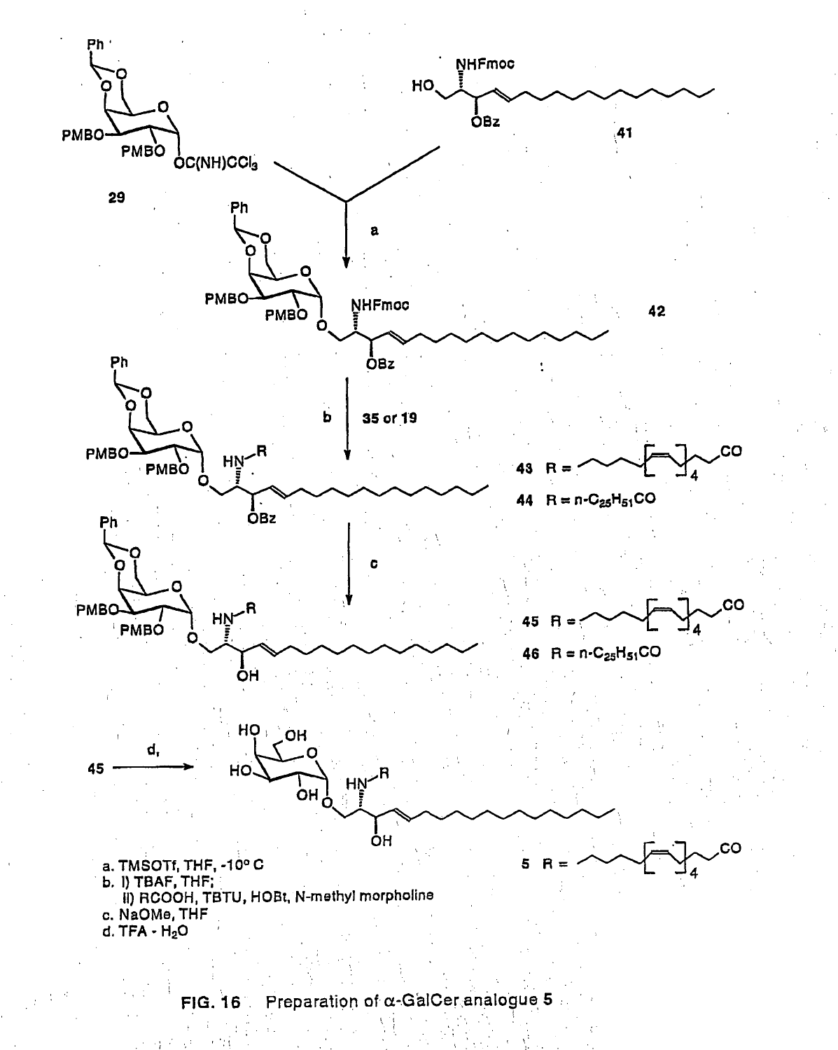

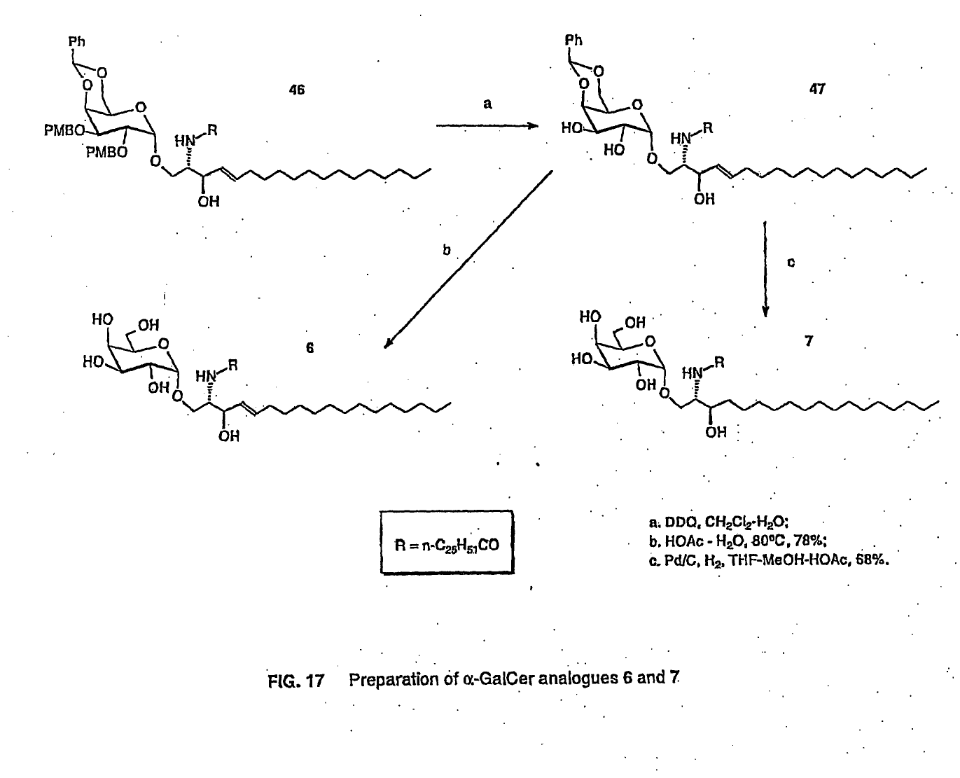

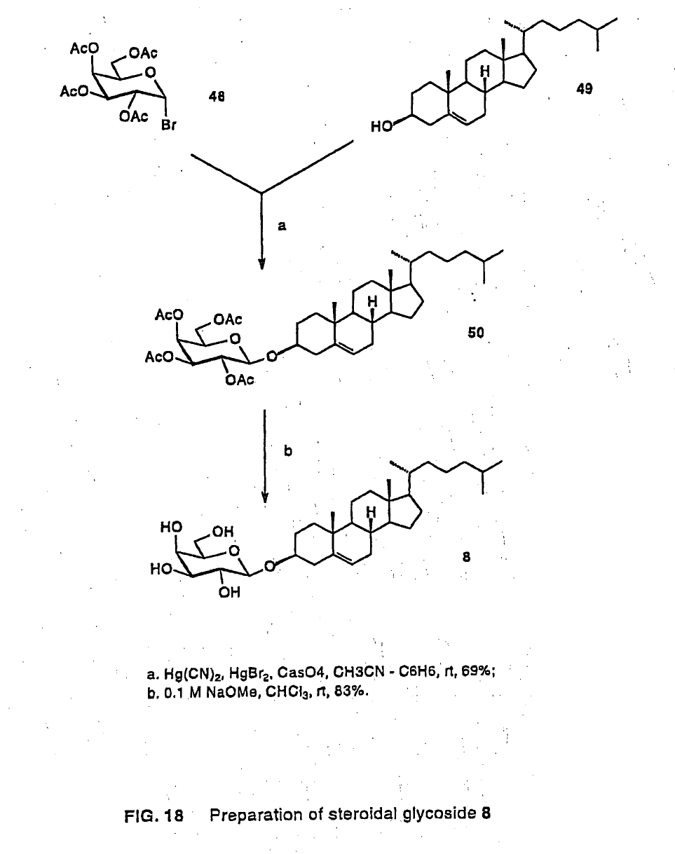

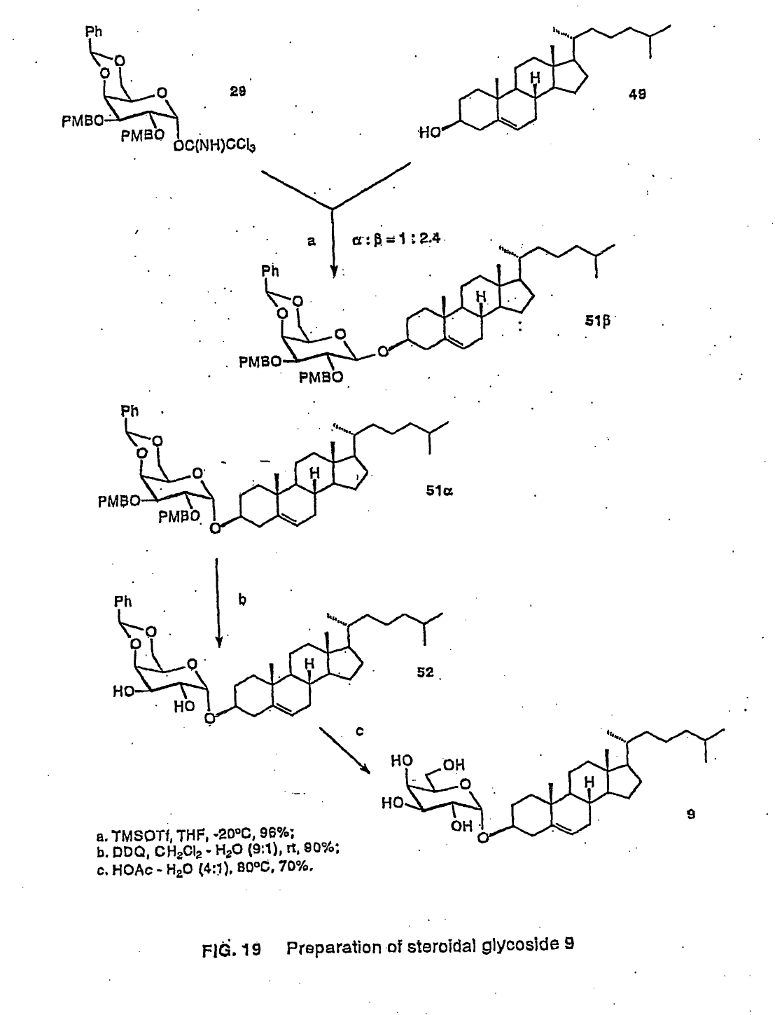

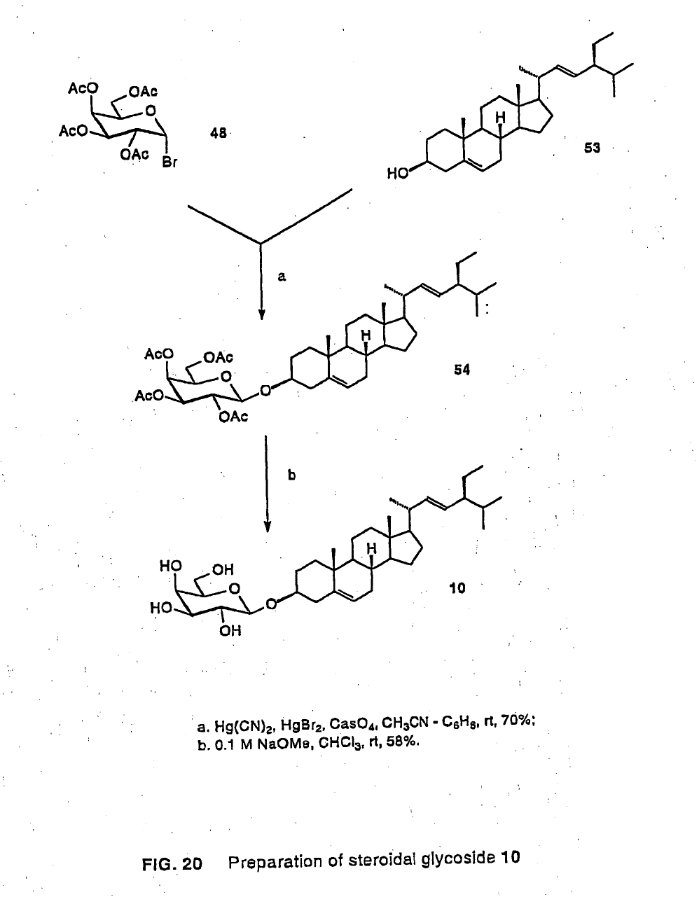

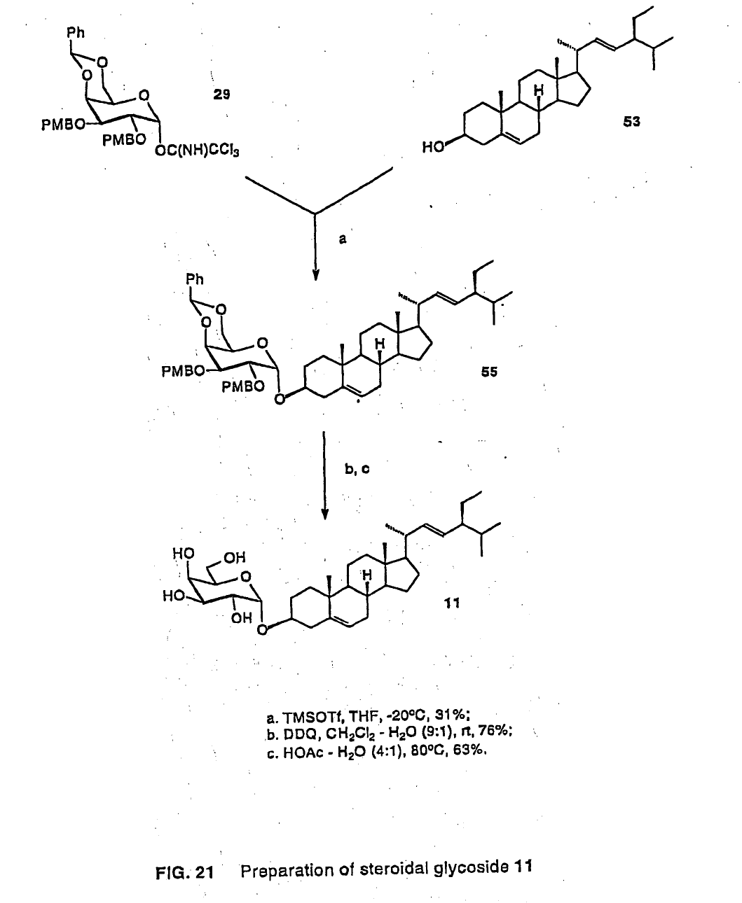

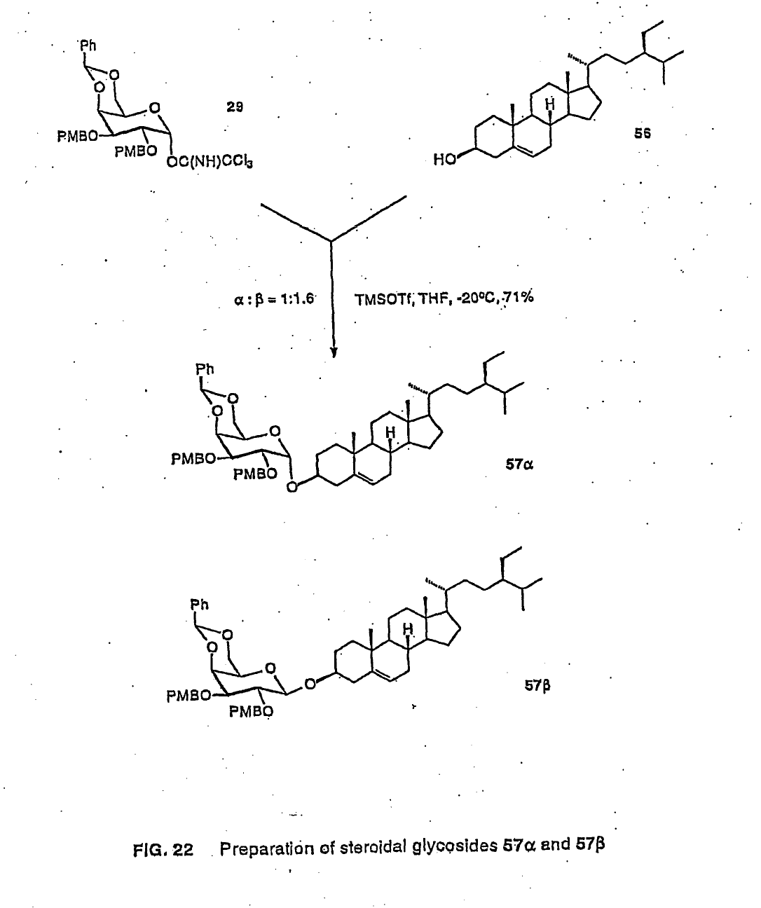

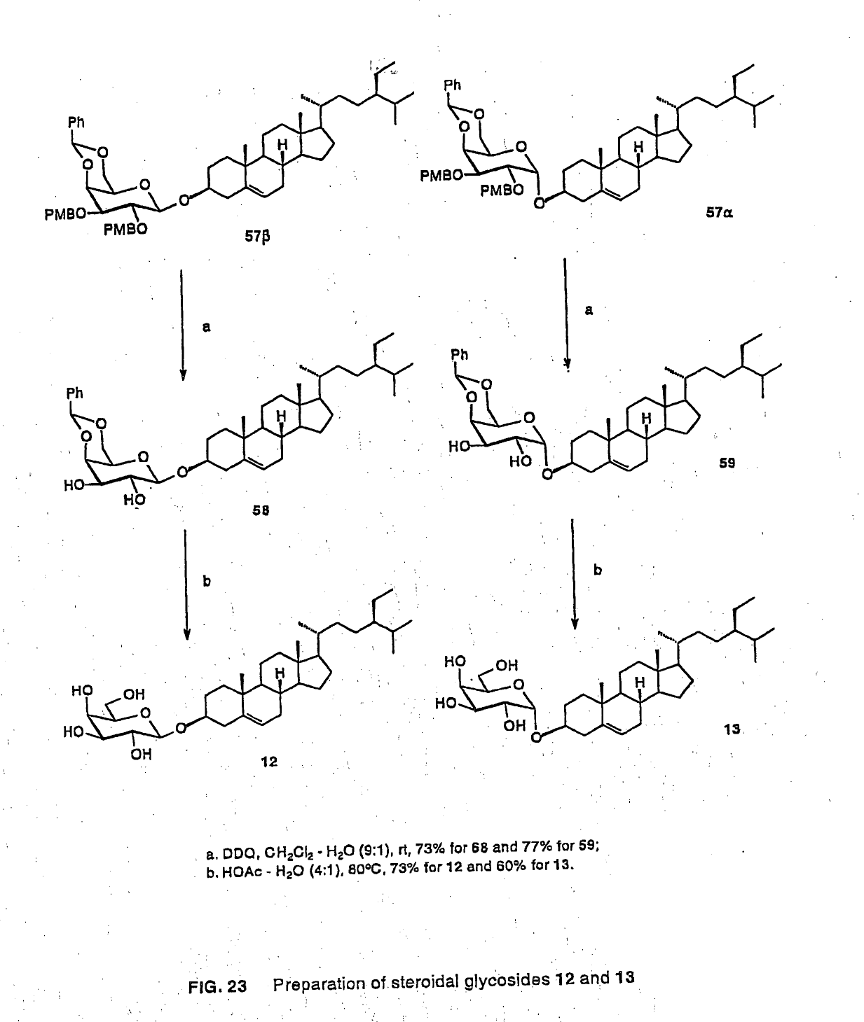

Please note the following correlation between the compound identifiers in activity FIGS. 24-30 and the compound numbers used in FIGS. 1-23 and the Examples.

038 =BC1-038 =compound 2

040 =BC1-040 =compound 3

041 =BC1-041 =compound 6

046 =BC1-046 =compound 8

047 =BC1-047 =compound 11

048 =BC1-048 =compound 10

049 =BC1-049 =compound 7

050 =BC1-050 =compound 1

BF 84=BF-1508-84 =compound 5

BF-1548-03=compound 4

051=BC1-051=compound 9

054=BC1-054=compound 12

### DETAILED DESCRIPTION OF THE PREFERRED EMBODIMENTS OF THE INVENTION

Utility

The compounds of the present invention, which are considered to be analogues of glycosylceramides, are useful as therapeutic agents, and, in particular, as antiviral, antimicrobial, antiparasitic and antitumor agents. They are useful by virtue of their immunomodulatory (immunostimulatory, immunosuppressive, or a combination thereof) and other biological activities. For example, alpha-GalCer exerts immunological activity by eliciting CD1-, especially CD1d-, restricted T cell responses. Beta-GalCer has anti-HIV activity as a result of the binding of that ligand to HIV gp120.

If the compound has immunomodulatory activity, it may have a Th1 bias, a Th2 bias, or no bias. Thus, alpha-galcer is unbiased, but the analogue OCH induces Th2 bias in NKT cells. See Miyamoto, et al., “A synthetic glycolipid prevents autoimmune encephalomyelitis by inducing Th2 bias of natural killer T cells,” Nature, 413: 531 (Oct. 4, 2001).

Gonzalez-Asequinolaza (2000, 2002) discloses the use of alpha-GalCer to activate Valphal4 natural killer T cells, which in turn mediate protection against murine malaria, an intracellular parasite. Sharif et al. (2001) has shown that this NKT cell activation also prevents the onset and recurrence of autoimmune type 1 diabetes.

In general, the glycosylceramide analogues of the present invention are useful as mimics or inhibitors of the known glycosylceramides. The uses of alpha and beta galactosyl ceramide have been discussed above.

Fucosylceramide has been identified as a tumor marker. See Yamada, et al., “Preferential expression of immunoreactive fucosylceramide in adenocarcinoma of the lung”, Cancer Research, Vol 52, Issue 16 4408-4412 (1992). Hence, a fucosylceramide analogue may be useful as an epitope or immunogen.

Lactosylceramide appears to be capable of inducing apoptosis. See Moore, et al., “Lactosylceramide-induced apoptosis in primary amnion cells and amnion-derived WISH cells”, J Soc Gynecol Investig. September-October 2002;9(5):282-89. See also van Blitterswijk, et al., “Sphingolipids related to-apoptosis from the point of view of membrane structure and topology”, Biochem Soc Trans. November 2001;29(Pt 6):819-24.

The glycosylceramide analogues of the present invention may be useful to activate, or to inhibit activation of, other glycolipid receptors. For example, bacterial adhesins often interact with host cell surface receptors to facilitate colonization. The glycosylceramide analogue could bind the cell surface receptor, blocking it off from the adhesin, or it could act as a decoy, so the adhesin binds harmlessly to it rather than to the receptor. Microbial or parasitic glycolipid receptors can bind to host cell membrane glycolipids; this likewise may be inhibited.

Glycolipid binding is the mechanism by which verotoxin targets renal endothelial cells to initiate the pathology which is characteristic of hemolytic uremic syndrome (HUS). The analogues of the present invention could be used to inhibit this binding. The glycosylceramide analogues of the present invention may be useful to activate, or inhibit activation of Toll-like receptors, especially TLR-1, -2 and -4. See generally Zuany-Amorin, et al., Nature rev., 1: 797-807 (October 2002).

A glycosylceramide analogue could also be used to elicit reduction in production and release of a natural glycosylceramide if the production and release is regulated by a negative feedback loop in which the produced glycosylceramide takes part, if the analogue could replace the natural molecule as a regulator. Several disorders are associated with excessive glycosylceramide.

Ceramide Replacement

In the compounds of the present invention, all or part of the ceramide of a naturally occurring glycosylceramide is modified or replaced with another moiety (optionally, the carbohydrate moiety is also modified or replaced). It is therefore of interest to consider in more detail the previously known GalCer analogues in which either the sphingoid base or the fatty acid moieties of GalCer have been modified.

In the compounds of the present invention, the R3 group corresponds to the fatty acid moiety of GalCer, while the —O-L(-N—R2)-A′ moiety corresponds to the sphingoid base.

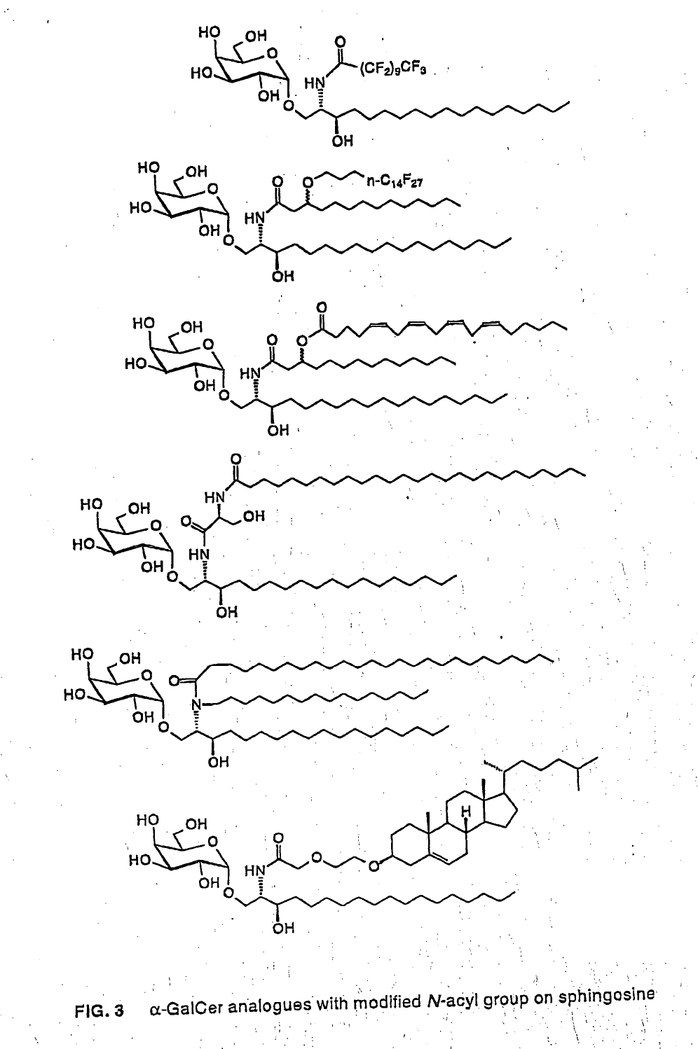

Kawano et al. (1997), FIG. 3, studied the effect of the different lengths of the fatty acyl chain and sphingosine base of alpha-GalCer on activation of Valphal4 NKT cells. Referring first to the fatty acyl chain, lengths of 26, 24, 14, and 2 (these include the carbonyl carbon) were tested, with a progressive reduction in activity as the chain length was decreased. The activity of the C14 analogue was a little less than 50% that of the C26 wild-type.

In all of the analogues, the sphingoid base was trihydroxylated (at 1, 3 and 4), and the amino group was at position 2. Only the chain length of the sphingoid base was varied, with values of 18, 15, and 11. Again, activity was directly related to chain length. The C15 analogue was about half as active as the wild-type C18, and the C11 analogue was about one-fourth as active.

Kawano et al. commented that the binding groove of the CD1d molecule has two large hydrophobic pockets, about 30 angstroms long and 10-15 wide. Kawano et al. estimated that the alpha GalCer with a C26 fatty acyl group and C18 sphingosine base was 34 angstroms long, with the subunit lengths being 28 (fatty acyl), 17 (sphingosine base), and 8 angstroms (sugar).

Morita et al. (JMC, 1995) prepared analogues of agelasphin-9b, and tested them for antitumor activity. The fatty acid moieties varied in chain length, over a range of 14-26. In some analogues, the C-2 was hydroxylated, and in others, it was not. The hydroxylation (Morita's Z position) did not seem to make much difference (compare AGL-548 with AGL-582, or AGL-512 with AGL-525). The chain length variation did make a difference, but even the analogue with the shortest FA moiety had some activity. Morita also varied the sphingoid base vis-a-vis hydroxylation at C-3 (his X position) and C-4 (his Y position), and chain length (16-28). Morita also made one analogue with a terminally branched sphingoid base (AGL-502). Antitumor activity was indifferent to the removal of the C-4 OH, but removal of the C-3 OH did reduce it. Chain length affected activity, with the maximum for C18. The branched analog AGL-502 was slightly more active than the isomeric analogue AGL-519. KRN-7000 is synonymous with AGL-582, and has a C16 fatty acid moiety, and a C28 sphingoid base moiety, the latter having 3-OH and 4-OH.

Brossay, et al., J. Immunol., 16: 5124-28 (1998) studied the effect of acyl chain length, and of the sphingoid base length and C3 and C4 hydroxylation, on presentation of the GalCer analogue by mCD1 or hCD1d to various mouse NKT cell hybridomas. The acyl chain length was varied from 2-26 (and also replaced altogether by an aniline ring), and the sphingoid base length from 11-18. Brossay found that even compound 587, with a two carbon acyl chain (but a normal 18 C length sphingoid base), was able to elicit a strong mCD1-dependent response. However, compound 591, with aniline in place of the acyl chain, was ineffective.

Likewise, the analogue 528, with a C11 sphingoid base, showed activity, although not as much as the C18 native form. Elimination of both the C-3 and C-4 hydroxyls (on the sphingoid base) abolished activity. However, the elimination of just the C-4 hydroxyl was tolerated, implying that it is the C-3 hydroxyl which is significant.

In Brossay's parallel study of presentation by hCD1d, the results of variation of the acyl chain length were similar. However, hCD1d was not able to present the analogue with the C11 sphingoid base; it did tolerate the shortening of the sphingoid base chain to C15. Also, hCD1d seemingly required retention of the C-4 hydroxyl.

### COMPOUNDS OF THE PRESENT INVENTION

There is no need to repeat here the generic structures already disclosed in the SUMMARY OF THE INVENTION. However, it is helpful to specify certain additional generic preferred embodiments.

Series A

In one series of embodiments (series A), the compounds of the present invention are represented by the following general. formula F-1A: 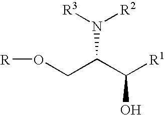
where R comprises a carboydrate moiety; R1 is primarily alkyl or -(spacer)-primarily alkyl; R2 is hydrogen, primarily alkanyl, or -(spacer)-primarily alkanyl; and R3 is

(A) -Z-R3″, where Z is a linker moiety consisting of one or more alkyl moieties and/or one or more spacers; and R3″ is a polyunsaturated moiety or an organic moiety comprising a steroidal moiety; or

(B) -Z-CF2-R3″, where Z is a linker moiety consisting of one or more alkyl moieties and/or one or more spacers; and R3″ is primarily alkanyl, or

(C) -Z(-R3b)-R3″, where Z is a trivalent linker moiety consisting of one or more alkyl moieties, including at least one secondary carbon, and/or one or more spacers; where R3b and R3″ are the same or different primarily alkyl moieties.

In preferred embodiments of series A, one or more of the following preferences apply, most preferably all of them (denoted series AA).

Preferably R is hexosyl, pentosyl, or nonosyl. If hexosyl, it may be deoxyhexosyl, aminohexosyl, or N-acetylaminohexosyl. If nonosyl it is preferably sialyl.

Preferably, if R1 contains non-alkyl moieties, they are preferably hydroxyl moieties, more preferably not more than one such moiety. Preferably, if R1 is unsaturated, it is monounsaturated, and more preferably the unsaturated bond is a double bond between C-1 and C-2, where C-1 is the carbon nearest the N of the formula.

Preferably R2, if organic, is —CH2-R2′ or —(C═O)—R2′, where R2′ is primarily alkanyl, and more preferably is alkanyl.

R3 preferably is defined by (A) as -Z-R3″ or by (C) as 13 Z(—R3b)-R3″.

In R3, Z is preferably a single spacerF, or is of the form spacerF-Z′-spacerL, where spacerF is the first spacer in Z, spacerL is the last spacer in Z, and Z′ is the remainder of Z, if any, and may comprise one or more spacers. SpacerF is preferably —C(═O)—. SpacerL is preferably —O— or —C(═O)—. Most preferably, Z is —C(═O)—, —C(═O)—CH2-CH(—O—)—, or —C(═O)—CH(—NH—C(═O)—)—CH2-O—.

In more preferred embodiments of series A, one or more of the following preferences applies, most preferably all of them (denoted series AAA).

Preferably R¹ is a substitution group selected from the group consisting of

—CH₂(CH₂ )ᵢCH₃₁

—CH═CH(CH₂)ᵢCH₃,

—CH(OH)(CH₂)ᵢCH₃,

—CH₂(CH₂)ᵢCH(CH₃)CH₂CH₃, and

—CH(OH)(CH₂)ᵢCH(CH₃)₂ , wherein i is an integer with values from 6 to 20; and

Preferably R² is a substitution group selected from the group consisting of

—H,

—CH₂(CH₂)ⱼCH₃, and

—CO(CH₂)ⱼCH₃, wherein j is an integer with values from 0 to 30.

Preferably R³ is a substitution group selected from the group consisting of

—CO(CF₂)ₘCF₃,

—COCF₂(CH₂)ₘCH₃,

—CO(CH₂)ₖ(CH═CHCH₂)₂(CH═CHCH₂)ₙ(CH₂)ₘCH₃, 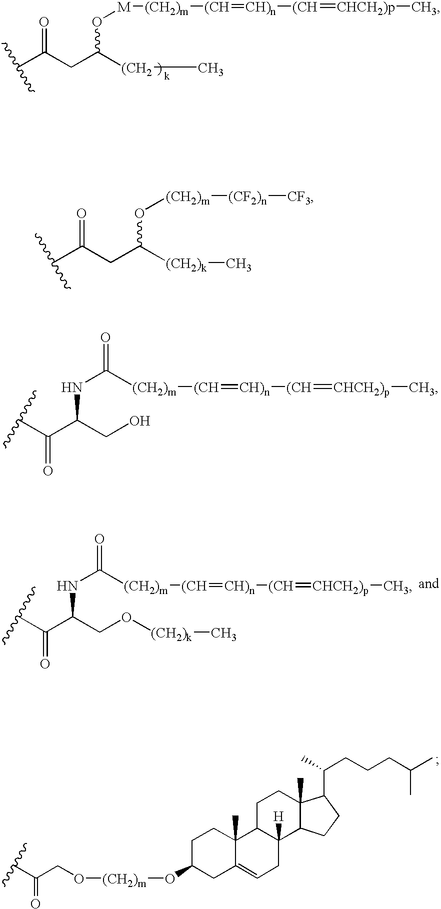
wherein M is CH₂ or CO; k and m are independent integers with values from 0 to 30, and n and p are independent integers with values from 0 to 10.

Even more preferably, said compound of series AAA is further defined by the following structure: 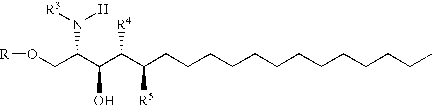
wherein R is chosen from structure I or II, 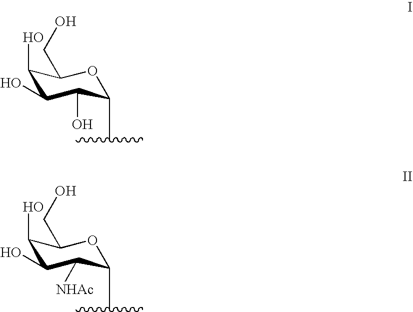
R⁴ is H or OH, and R⁵ is H; or R⁴ and R⁵ form a double bond.

Most preferably, this series AAA compound has the structure

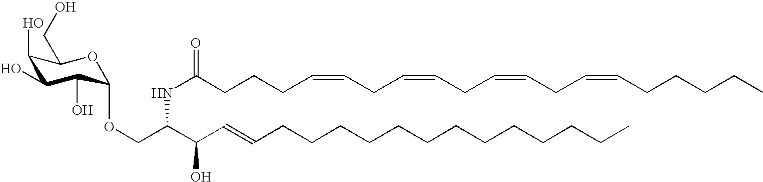

We may further define a separate series AF of the general formula 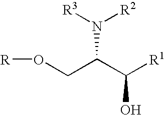
where R, R1 and R2 take on the various preferred values set forth for series A, AA and AAA, and where R3 is of the form -(Z)₀₋₁-CF2-R3′, Z is a single spacer, -spacer-CH2-spacer-, or a spacer cluster, and R3′ is a primarily alkyl moiety. It will be appreciated that this series also belongs to formula F-F.

Preferably, in series AF, R3 is —CO(CF₂)ₘCF₃ or —COCF₂(CH₂)ₘCH₃.

Series B

In a second series of embodiments (series B), the compounds of the present invention are represented by the following formula F-4B: 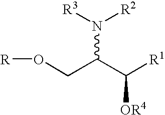
wherein R comprises a carbohydrate moiety;

R1 is hydrogen or -Z1-R1′, where Z1 is a linker moiety consisting of one or more spacers and, optionally, one or more alkanyl moieties; and where R1′ is primarily alkyl;

R2 is hydrogen, primarily alkanyl, or -(spacer)-primarily alkanyl;

R3 is -Z3-R3′, where Z3 is a linker moiety consisting of one or more alkanyl moieties and/or one or more spacers; and where R3′ is primarily alkyl, or is an organic moiety comprising a steroidal moiety; and

R4 is hydrogen or -Z4-R4′, where Z4 is a linker moiety consisting of one or more alkanyl moieties and/or one or more spacers; and where R4′ is primarily alkanyl.

In preferred embodiments of series B, one or more of the following preferences apply, most preferably all of them (denoted series BB).

Preferably R is hexosyl, pentosyl, or nonosyl. If hexosyl, it may be deoxyhexosyl, aminohexosyl, or N-acetylaminohexosyl. If nonosyl it is preferably sialyl.

Z1 is preferably -X-Y-Z, where X and Z are independently —CH2- or —C(═O)—, and Y is —O—, —NH—, or —S—.

R1′ may be a saturated moiety, a monounsaturated moiety, or a polyunsaturated moiety. If it contains non-alkyl moieties, they are preferably hydroxyl moieties, more preferably not more than one such moiety.

R2, if organic, preferably is —CH2-R2′ or —(C═O)—R2′, where R2′ is primarily alkanyl, and more preferably is alkanyl.

R3 is preferably at least partially fluorinated, or comprises a polyunsaturated moiety, or comprises a steroidal moiety.

Z3 is preferably a single spacerF, or is of the form spacerF-Z3′-spacerL, where spacerF is the first spacer in Z3, spacerL is the last spacer in Z3, and Z3′ is the remainder of Z3, if any, and may comprise one or more spacers. SpacerF is preferably —C(═O)—. SpacerL is preferably —O— or —C(═O)—. Most preferably, Z3 is —C(═O)—, —C(═O)—CH2-CH(—O—)—, or —C(═O)—CH(—NH—C(═O)—)—CH2-O—.

Z4 is preferably —CH2- or —C(═O)—. If R4 contains non-alkyl moieties, they are preferably hydroxyl moieties, more preferably not more than one such moiety.

In more preferred embodiments of series BB, one or more of the following preferences apply, most preferably all of them (denoted series BBB).

R¹ preferably is a substitution group selected from the group consisting of

—H,

—X—Y-Z-(CH₂)ᵢCH₃,

—X—Y-Z-(CH₂)ᵣ(CH═CHCH₂)_{q}(CH₂)ᵢCH₃, and

—X—Y-Z-(CH₂)ᵣCH(OH)(CH2)ᵢCH₃,

wherein X and Z are independently CH₂ or CO, and Y is O, NH, or S; i and r are independent integers with values from 0 to 30, and q is an integer with values from 1 to 10;

R² preferably is a substitution group selected from the group consisting of

—H,

—CH₂(CH₂)ⱼCH₃, and

—CO(CH₂)ⱼCH₃, wherein j is an integer with value from 0 to 30;

R³ preferably is a substitution group selected from the group consisting of

—CO(CH₂)ₘCH(OH)(CH₂)ₖCH₃

—CO(CF₂)ₘCF₃,

—COCF₂ (CH₂)ₘCH₃,

—CO(CH₂)ₖ(CH═CHCH₂)ₙ(CH₂)ₘCH₃ , and

a structure of the following: 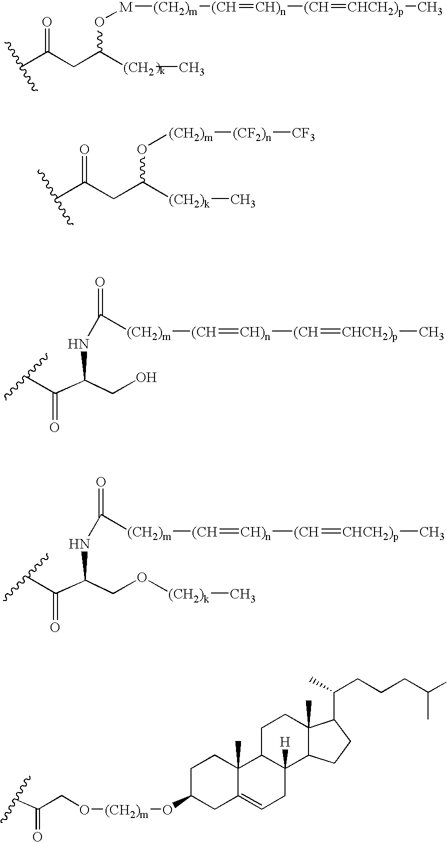
wherein M is CH₂ or CO; k and m are independent integers with values from 0 to 30, and n and p are independent integers with values from 0 to 10; and

R⁴ preferably is a substitution group selected from the group consisting of

—H,

-M-(CH₂)ₛCH(OH)(CH₂)ₜCH₃, and

-M-CH(CH₂OH)(CH₂)ₛCH₃

wherein M is CH₂ or CO; and s and t are independent integers with values from 0 to 30.

Within series B, molecules wherein R¹ and R² are hydrogen atoms, R3 is defined as for series B generally, and R is an α-D-galactopyranosyl residue, are of particular interest. These α-GalCer analogues are characterized by the total replacement of the ceramide moiety with a fatty acyl moiety derived from serinol.

More preferably, the series BBB compound is further defined by the following structure: 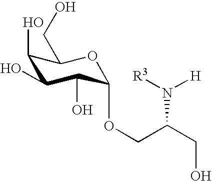
where R3 is as previously defined

Even more preferably, the R3 therein has one of the following structures:

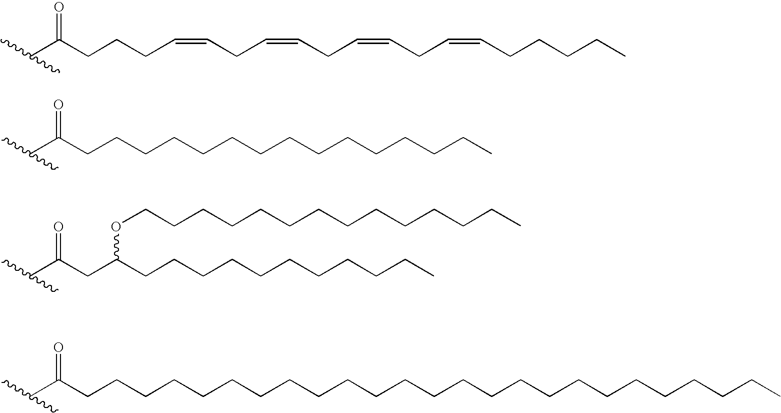

Most preferably, the series BBB compound has the structure 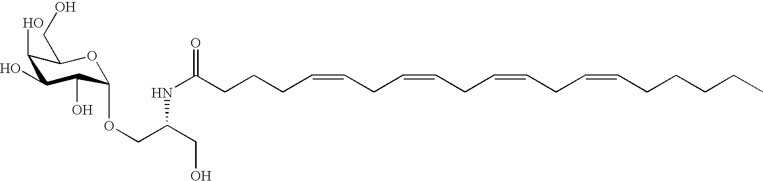
Series C

In a third series of embodiments (series C), the compounds of the present invention are depicted by the following general formula F-8C. 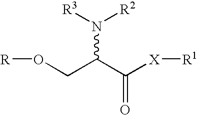
wherein R comprises a carbohydrate moiety; R1 is hydrogen or is an organic moiety which is substantially linear and primarily alkyl; X denotes —O—, —NH— or —S—; R2 is hydrogen, primarily alkanyl, or -(spacer)-primarily alkanyl; and R3 is -Z3-R3′, where Z3 is a linker moiety consisting of one or more alkanyl moieties and/or one or more spacers; and where R3′ is primarily alkyl, or is an organic moiety comprising a steroidal moiety.

In preferred embodiments of series C, one or more of the following preferences apply, most preferably all of them (denoted series CC).

Preferably R is hexosyl, pentosyl, or nonosyl. If hexosyl, it may be deoxyhexosyl, aminohexosyl, or N-acetylaminohexosyl. If nonosyl it is preferably sialyl.

R1 may be a saturated moiety, a monounsaturated moiety, or a polyunsaturated moiety. If it contains non-alkyl moieties, they are preferably hydroxyl moieties, more preferably not more than one such moiety.

R2, if organic, preferably is —CH2-R2′ or —(C═O)-R2′, where R2′ is primarily alkanyl, and more preferably is alkanyl.

R3 is preferably at least partially fluorinated, or comprises a polyunsaturated moiety, or comprises a steroidal moiety.

Z3 is preferably a single spacerF, or is of the form spacerF-Z3′-spacerL, where spacerF is the first spacer in Z3, spacerL is the last spacer in Z3,and Z3′ is the remainder of Z3, if any, and may comprise one or more spacers. SpacerF is preferably —C(═O)—. SpacerL is preferably —O— or —C(═O)—. Most preferably, Z3 is —C(═O)—, —C(═O)—CH2-CH(—O—)—, or —C(═O)—CH(—NH—C(═O)—)—CH2-O—.

In more preferred embodiments of series CC, one or more of the following preferences apply, most preferably all of them (denoted series CCC).

R¹ preferably is a substitution group selected from the group consisting of

—H,

—(CH₂)ᵣ(CH═CHCH₂)_{q}(CH₂)ᵢCH₃, and

—(CH₂)ᵣCH(OH)(CH₂)ᵢCH₃,

wherein r and i are independent integers with values from 0 to 30, and q is an integer with values from 0 to 10.

R² preferably is a substitution group selected from the group consisting of

—H,

—CH₂(CH₂)ⱼCH₃, and

—CO(CH₂)ⱼCH₃,

wherein j is an integer with values from 0 to 30.

R³ is a substitution group selected from the group consisting of

—CO(CH₂)ₘCH(OH)(CH₂)ₖCH₃

—CO(CF₂)ₘCF₃,

—COCF₂ (CH₂)ₘCH₃,

—CO(CH₂)ₖ(CH═CHCH₂)ₙ(CH₂)ₘCH₃, and

a structure of the following: 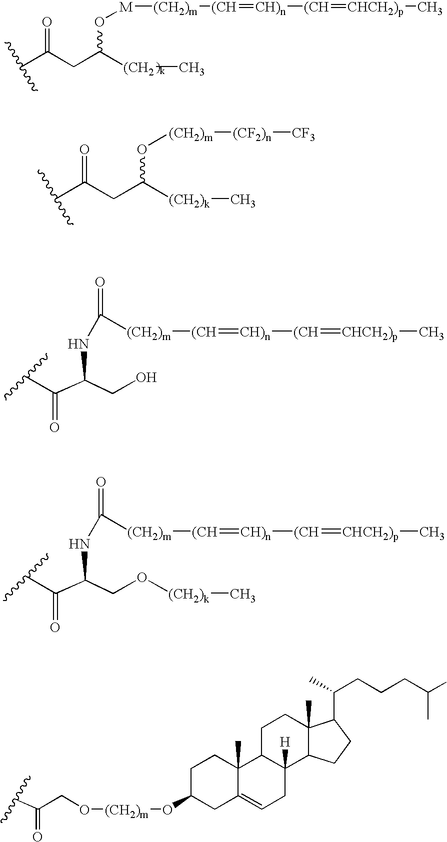
wherein M is CH₂ or CO; k and m are independent integers with values from 0 to 30, and n and p are independent integers with values from 0 to 10.

These series CCC compounds may be characterized as analogues in which ceramide is replaced by serine-based fatty acyl derivatives.

More preferably, said series CCC compound is further defined by the following: 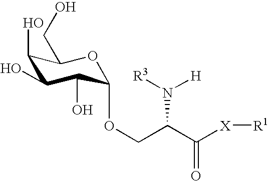
wherein R1, R3 and X are as previously defined.
Series D

In a fourth series of embodiments (series D), the compounds of the present invention have the following general structure F-10D: 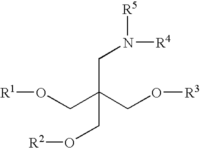
wherein R¹ and R² is are independently selected from the group consisting of hydrogen, an organic moiety comprising a carbohydrate moiety, and an organic moiety comprising another Pet unit, and at least one of R¹ and R² is not hydrogen; R3 is a substantially linear and primarily alkyl moiety; R4 is hydrogen, or a substantially linear, primarily alkanyl moiety; and R5 is -Z5-R5′, where Z5 is a linker moiety consisting of one or more alkyl moieties and/or one or more spacers; and where R5′ is primarily alkyl, or is an organic moiety comprising a steroidal moiety.

In preferred embodiments of series D, one or more of the following preferences apply, most preferably all of them (denoted series DD).

If R1 or R2 is a carbohydrate moiety, then preferably the carbohydrate moiety (chosen independently) is hexosyl, pentosyl, or nonosyl. If hexosyl, it may be deoxyhexosyl, aminohexosyl, or N-acetylaminohexosyl. If nonosyl it is preferably sialyl.

R3 may be a saturated moiety, a monounsaturated moiety, or a polyunsaturated moiety. If it contains non-alkyl moieties, they are preferably hydroxyl moieties, more preferably not more than one such moiety.

R4, if organic, preferably is —CH4-R4′ or —(C═O)—R4′, where R4′ is primarily alkanyl, and more preferably is alkanyl.

R5 is preferably at least partially fluorinated, or comprises a polyunsaturated moiety, or comprises a steroidal moiety.

Z5 is preferably a single spacerF, or is of the form spacerF-Z5′-spacerL, where spacerF is the first spacer in Z5, spacerL is the last spacer in Z5, and Z5′ is the remainder of Z5, if any, and may comprise one or more spacers. SpacerF is preferably —C(═O)—. SpacerL is preferably —O— or —C(═O)—.

Most preferably, Z5 is —C(═O)—, —C(═O)—CH2-CH(—O—)—, or —C(═O) —CH(—NH—C(═O)—) —CH2-O—.

In more preferred embodiments of series DD, one or more of the following preferences apply, most preferably all of them (denoted series DDD).

R³ preferably is a substitution group selected from the group consisting of

—H,

—(CH₂)ᵥCH₃,

—CO(CH₂)ᵥCH₃,

—CO(CH₂)ᵤ(CH═CHCH₂)ᵥ(CH₂)ₜCH₃,

—(CH₂)ᵤCH(OH)(CH₂)ₜCH₃, and

—CO(CH₂)ᵤCH(OH)(CH₂)ₜCH₃,

wherein t and u are independent integers with values from 0 to 30, and v is an integer with values from 1 to 10.

R⁴ preferably is a substitution group selected from the group consisting of

—H,

—CH₂(CH₂)ₛCH₃, and

—CO(CH₂)ₛCH₃ wherein s is an integer with values from 0 to 30.

R⁵ is a substitution group selected from the group consisting of

—CO(CH₂)ₘCH₃,

—CO(CH₂)ₘCH(OH)(CH₂)ₖCH₃

—CO(CF₂)ₘCF₃,

—COCF₂ (CH₂)ₘCH₃,

—CO(CH₂)ₖ(CH═CHCH₂)ₙ(CH₂ )ₘCH₃, and

a structure of the following: 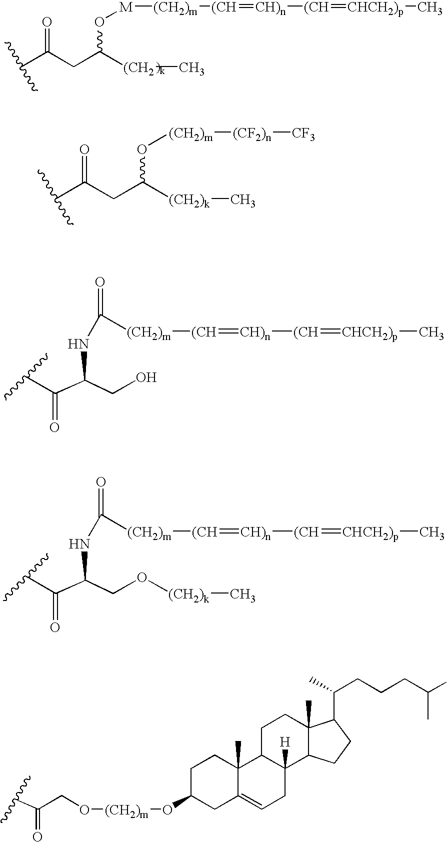
wherein M is CH₂ or CO; k and m are independent integers with values from 0 to 30, and n and p are independent integers with values from 0 to 10.

More preferably, the series DDD compound is further defined by the following: 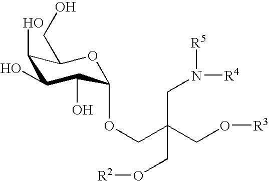
wherein

R² is hydrogen or α-D-galactopyranosyl residue (I), 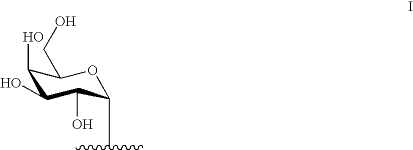
and R3, R4 and R5 are as previously defined.
Series E

In a fifth series of embodiments (series E), the compounds of the present invention are terpenoid, steroid or alkaloid galactosides, as shown by the following structure F-12E: 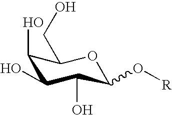
wherein R is a residue of a steroid, terpenoid, or an alkaloid.

It will be appreciated that if terpenoidal, R may be a residue of an iridoid, sesqiterpenoid, diterpenoid, triterpenoid.

In a preferred embodiment of the series E compounds, group R is chosen from the following: 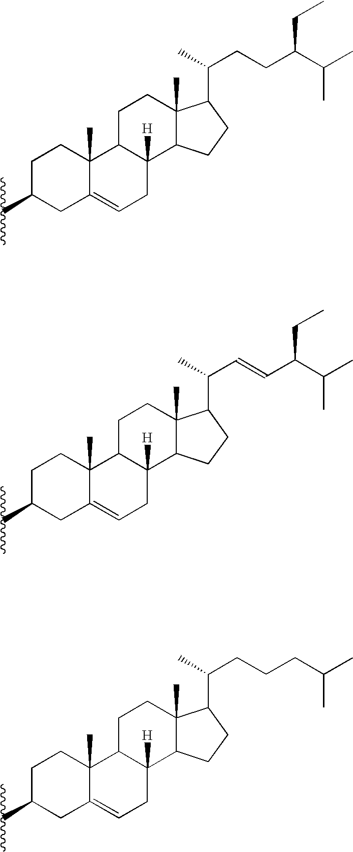
Synthetic Intermediates

The present invention also discloses novel glycosyl donors that are suitable to construct α-linked galactopyranosides. The galactosyl donors are illustrated by the following structure: 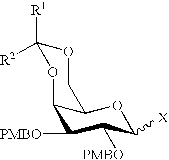
wherein X represents a leaving group including, but not limited to, halogen, —OC(NH)CCl₃, —SR, SO₂R, —O(CH₂)₃CH═CH₂, —P(OR)₂, and P(O) (OR)₂ wherein R is an alkyl or aromatic group.

These galactosyl donors are particularly useful for the preparation of α-GalCer analogues which contain carbon-carbon double bond(s) in the ceramide moiety, because the protecting groups on the galactose residue can be removed without affecting the carbon-carbon double bond(s) in the aglycone.

Synthetic Methods

The present invention also includes a novel process of making α-GalCer analogues (mimics) that contain at least one double bond in the aglycone. The process comprises the following steps:

a) The glycosylation reaction is carried out, in the presence of a Lewis acid as a catalyst, by using the following glycosyl donor: 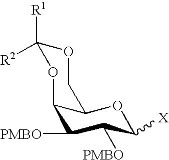
wherein

X represents a leaving group including, but not limited to, halogen, —OC(NH)CCl₃, —SR, SO₂R, —O(CH₂)₃CH═CH₂, —P(OR)₂, and P(O) (OR)₂ wherein R is an alkyl or aromatic group;

R¹ and R² are independently hydrogen atom, alkyl group, or aromatic group;

and the following glycosyl acceptor: 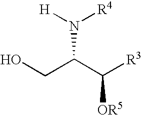
wherein

R³ is hydrogen, or an alkyl or alkenyl group, substituted or unsubstituted;

R⁴ is an amine protecting group or an fatty acyl group; and

R⁵ is a hydroxyl protecting group;

to provide the following glycoside: 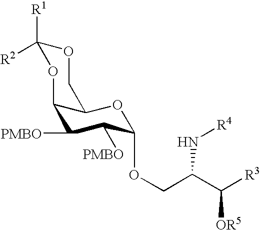
wherein

R¹ to R⁵ are defined as above.

b) The amine protecting group R⁴ (when applicable) in the product formed in step a) is removed to give the following free amine: 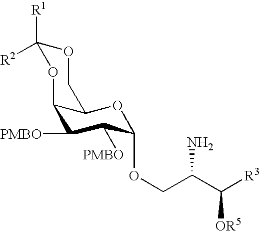
wherein

R¹ to R⁵ are defined as above. c) An fatty acyl group is introduced at amine position of the product formed in step b) in the presence of a conventional coupling reagent to give: 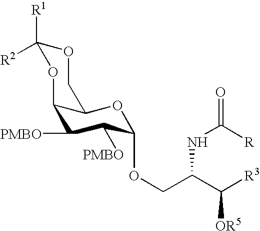
wherein

R is an alkyl or alkenyl group, substituted or unsubstituted, and R¹ to R⁵ are defined as above.

d) The protecting groups R⁵, PMB, and R¹R²CH acetal/ketal at 4,6-O-position in the product formed in step c) are deprotected in a non-preferential order to give the α-GalCer analogue of the following structure: 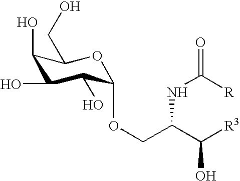
wherein

R and R³ are independently alkyl groups, with at least one group carrying at least one double bond.

In the process, the removal of any one or all of the protecting groups (R⁵, PMB and R¹R²CH acetal/ketal) described in step d) may be carried out before step b) to provide the same final product of α-GalCer analogues.

### DEFINITIONS

Carbohydrate Moiety

The analogues of the present invention comprise a carbohydrate moiety, and/or at least one Pet unit. The term “carbohydrate” (sugar) includes monosaccharides, oligosaccharides and polysaccharides, as well as substances derived from the monosaccharides by reduction of the carbonyl group (alditols), by oxidation of one or more terminal groups to carboxylic acids, or by replacement of one or more hydroxy groups by a hydrogen atom, an amino group, a thiol group, or similar heteroatomic groups. It also include derivatives of the foregoing.

In preferred embodiments, the carbohydrate is a mono, di-, tri-, tetra-, penta- or hexasaccharide.

When the carbohydrate moiety is attached to another moiety, and is not a monosaccharide, the sugar unit closest to the foreign moiety is called the inner or proximal sugar. If a carbohydrate moiety is attached to several non-carbohydrate moieties, the definition of inner or proximal sugar is based on proximity to the largest of the attached non-carbohydrate moieties.

Monosaccharides (Sugar Units)

Parent monosaccharides are polyhydroxy aldehydes (H[CHOH]ₙ—CHO) or polyhydroxy ketones (H—[CHOH]ₙ—CO—[CHOH]ₘ—H) with three or more carbon atoms. The term —monosaccharide unit”, “carbohydrate unit” or “sugar unit” refers to a residue of a monosaccharide, including the derivatives of monosaccharides contemplated herein.

Each monosaccharide unit is preferably a triose (e.g., glyceraldehyde), tetrose (e.g., erythrose, threose), pentose (e.g., ribose, arabinose, xylose, lyxose), hexose (e.g., allose, altrose, glucose, mannose, gulose, idose, galactose, talose), heptose, octose, nonose or decose. More preferably it is a pentose or hexose, or the nonose sialic acid. The term hexosyl includes deoxyhexosyl, aminohexosyl, N-acetylaminohexosyl, and other derivatives of the basic hexosyl structure that do not alter the number of carbon atoms.

Each monosaccharide unit may be an aldose (having an aldehydic carbonyl or potential aldehydic carbonyl group) or a ketose (having a ketonic carbonyl or potential ketonic carbonyl group). (Fructose is an example of a ketose.) The monosaccharide unit further may have more than one carbonyl (or potential carbonyl) group, and hence may be a dialdose, diketose, or aldoketose. The term “potential aldehydic carbonyl group” refers to the hemiacetal group arising from ring closure, and the ketonic counterpart (the hemiketal structure).

The ketoses include the tetrose erythrulose, the pentoses ribulose and xylulose, and the hexoses pscicose, fructose, sorbose and tagatose, and their derivatives. These have both D- and L-forms.

The aldoses are of particular interest and include the triose glyceraldehyde, the tetroses erythrose and threose, the pentoses ribose, arabinose, xylose and lyxose, and the hexoses allose, altrose, glucose, mannose, gulose, idose, galactose and talose, and their derivatives. These have both D- and L-forms.

The monosaccharide unit may be a cyclic hemiacetal or hemiketal. Cyclic forms with a three membered ring are oxiroses; with four, oxetoses, with five, furanoses; with six, pyranoses; with seven, septanoses, with eight, octaviruses, and so forth. The locants of the positions of ring closure may vary. Note that in the more common cyclic sugars, the ring consists of one ring oxygen, the remaining ring atoms being carbon; hence, in pyranose, there is one ring oxygen and five ring carbons.

The monosaccharide unit may further be a deoxy sugar (alcoholic hydroxy group replaced by hydrogen), amino sugar (alcoholic hydroxy group replaced by amino group), a thio sugar (alcoholic hydroxy group replaced by thiol, or C═O replaced by C═S, or a ring oxygen of cyclic form replaced by sulfur), a seleno sugar, a telluro sugar, an aza sugar (ring carbon replaced by nitrogen), an imino sugar (ring oxygen replaced by nitrogen), a phosphano sugar (ring oxygen replaced with phosphorus), a phospha sugar (ring carbon replaced with phosphorus), a C-substituted monosaccharide (hydrogen at a non-terminal carbon atom replaced with carbon), an unsaturated monosaccharide, an alditol (carbonyl group replaced with CHOH group), aldonic acid (aldehydic group replaced by carboxy group), a ketoaldonic acid, a uronic acid, an aldaric acid, and so forth. Amino sugars include glycosylamines, in which the hemiacetal hydroxy group is replaced.

Derivatives of these structures include O-substituted derivatives, in which the alcoholic hydroxy hydrogen is replaced by something else. Possible replacements include alkyl, acyl, phosphate, phosphonate, phosphinate, and sulfate. Likewise, derivatives of amino sugars include N-substituted derivatives, and derivatives of thio sugars include S-substituted derivatives.

Sialic acid, also known as N-acetyl neuraminic acid (NANA), is of particular interest. It is the terminal sugar on several tumor-associated carbohydrate epitopes. It is a pyranose, and a nonose with a methyl-CONH— substitution at C-5.

In biosynthesized glycosphingolipids, the most common sugar units are glucose, galactose, fucose, mannose, GalNAc, GlcNAc, and sialic acid. The inner sugar is usually galactose or glucose.

Preferably, the compounds of the present invention comprise one, two, three four or five sugar or Pet units, the two being considered interchangeable for this purpose. Preferably, each sugar unit is, independently, a hexose or a pentose. The hexose may be, without limitation, a deoxyhexose, aminohexose, or N-acetylaminohexose. Alternatively, the sugar unit may be a sialic acid.

In some embodiments, the carbohydrate moiety is chosen to confer the ability to elicit natural killer cell activity. Kawano et al. (1997) compared the ability of ceramide, and various glycosylceramides, to elicit natural killer cell activity. Specifically, they studied CDld-restricted, TCR-mediated activation of Vα14 NKT cells. The active molecules tested were α-GalCer, α-GlcCer, 3,4-deoxy α-GalCer, Galα1-6Galα1-1′Cer, Galα1-6Glcα1-1′Cer, Galα1-2Galα1-1′Cer, Galβ1-3Galα1-1′Cer. The inactive molecules were ceramide, β-GalCer, α-ManCer. and Galα1-4Glcβ1-1′Cer. The most active molecule was α-GalCer, with the other active molecules being roughly 20-70% as active at DC of 2E4 cells.

Thus, in a preferred embodiment, the “inner” sugar has an alpha anomeric configuration and an equatorially configured 2-hydroxyl group (as in Gal and Glc; Man has axial configuration).

Ijima et al. (1998) pretreated dendritic cells (DC) with various glycosyl ceramides, and determined the degree to which the pretreated DCs stimulated the proliferation of speen cells. Thus, this was a mixed leucocyte reaction with dendritic cells as the stimulator cells and spleen cells as the responder cells. The three beta-glycosyl ceramides tested were inactive, whereas the corresponding alpha-anomers were active. They tested one alpha-furanosyl ceramide, AGL-574; it lacked activity. This implied that the pyranose form was desirable for MLR activity. One of Ijima's active GalCer analogues was AGL-517. AGL-575, a 2″-des-OH analogue of AGL-517 lacked-activity, implying that retention of the 2″-OH on the Gal unit was desirable. Shifting the 4″-OH in AGL-517 from the axial to the equatorial position (AGL-563) reduced, but did not abolish, activity.

Uchimura et al. (1997) studied the immunostimulatory activity of various mono or diglycosylated alpha-galactosylceramides isolated from Okinawan marine sponge. (Note that these comprise di- or trisaccharides, respectively.) The 2″-monoglycosylated alpha galactosylceramide was more potent than the 3″-monoglycosylated alpha GalCer, implying that a free 3″hydroxyl group plays a more important role in the studied immunostimulatory activity than a free 2″-hydroxyl group. However, Constantino et al. had previously concluded that 2″ monoglycosylation of the alpha-GalCer was undesirable because hist derivatives did not show immunostimulatory effects on the proliferation of lymph node cells. Uchimura et al. confirmed that the effects of 2″ monoglycosylated alpha GalCers on spleen cells and lymph node cells were quite different. In another study, this time of chemically synthesized 6″ monoglycosylated alpha-GalCer and 4″ or 6″ monoglycosylated alpha-GluCer, Uchimura et al. reported (1) the 6″OH group of alpha-galcers has no effect, (2) the configuration of 4″ position of the inner pyranose moiety is important, (3) the 4″ group is more important than the 6″ group.

Sakai, et al., Organic Lett. 1: 359-61 (1999) reported that AGL-597, a biotinylated analogue of KRN7000, was substantially more potent than the latter. The biotinylation was of the terminus of the fatty acyl moiety.

In other embodiments, the purpose of the sugar is to bind gp 120 in such manner as to confer anti-HIV-1 activity, analogous to the activity of betaGalCer. Hence, the carbohydrate moiety may be betaGal, or one whose inner sugar is betaGal.

Pet Units

Pentaerythritol (Pet) has a the five carbon backbone (core) which features a central carbon, singly bonded to four peripheral carbons:

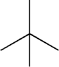

These carbons are, in turn, be joined to other moieties.

Thus, the analogs of the present invention may comprise the structure

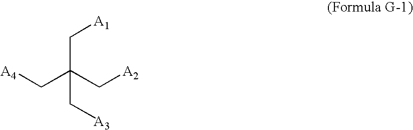

where A1-A4 are hereafter defined. Each of A1-A4 may be considered a “primary branch” of the analog.

In a preferred embodiment, A₁ is Y₁Z₁, A₂ is Y₂Z₂, A₃ is Y₃Z₃ and A₄ is Y₄Z₄, where Y₁-Y₄ are spacers as hereafter defined. Preferably, each of Z₁-Z4 is, independently, selected from the group consisting of hydrogen, an organic group, or a group which in conjunction with the adjacent Y group forms a phosphate, sulfate or borate. To put it another way, preferably each of Z1-Z4 is independently selected from the group consisting of hydrogen, —P(═O)(OH)OH, —C(═O)OH, —S(═O)(═O)OH, —B(OH)OH, or an organic group. Preferably, each of these organic groups has not more than 200 atoms other than hydrogen, more preferably, not more than 150, still more preferably, not more than 100.

The Pet unit may be considered to be the Pet backbone (core) as defined above, together with the Y₁-Y₄ groups which correspond to or replace the hydroxyl oxygens of unmodified Pet:

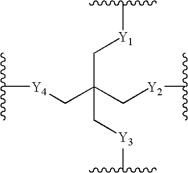

Pentaerythritol can be considered to be the compound of general formula I in which A1-A4 are all —OH. Equivalently, it is the compound of that formula in which Y1-Y4 are all —O— and R1-R4 are all —H.

While pentaerythritol per se is not one of the analogs of the present invention, the latter does contemplate the incorporation of spacers Y1-Y4 which are —O— or analogs thereof.

In a preferred embodiment, each of spacers Y1-Y4 is independently selected from the group consisting of —(CH₂)ₙO—, —(CH₂)ₙS—, and —(CH₂)ₙN<, where n is, independently, 0 to 4. More preferably, each of these spacers is —O—, —S— or —N< (i.e., n is 0). Even more preferably, each of these spacers is —O— or —N<, and the latter still more preferably is —NH—.

Most preferably, either (a) all of these spacers are —O—, or (b) one spacer is —NH— and the other spacers are —O—.

When the Pet unit is serving as a sugar replacement, there are no further constraints on spacers Y1-Y4. However, when the Pet unit is serving as a ceramide replacement, one spacer must be —N<, and is preferably —NH—. The other spacers then are preferably —O—;

Spacers

A spacer is defined as a divalent moiety selected from the group consisting of —NR*- (where R* is hydrogen, or alkanyl of 1-4 carbons), —C(═O)—, —C(═S)—, —O— or —S—. R* is preferably hydrogen or methyl, most preferably hydrogen.

Spacer Clusters

Spacers may occur consecutively, in which case they form a substructure called a “spacer cluster”. Preferably, a spacer cluster is two, three or four consecutive spacers.

Allowed Spacer Clusters

In the compounds of the present invention, a spacer cluster is allowed only if, within the cluster, spacer nitrogen is not immediately adjacent to spacer nitrogen, spacer carbonyl carbon is not immediately adjacent to spacer carbonyl carbon, and spacer chalcogen is not immediately adjacent to spacer chalcogen.

Substantially Linear

A group is substantially linear if (1) all of the non-hydrogen atoms form a single chain, or (2) if the longest chain formed by its non-hydrogen atoms is more than twice the length of the longest non-overlapping chain formed by the remainder of the non-hydrogen atoms. Thus, in —(CH2)6-CH(—CH2CH3)-CH3, the longest non-H chain is 8 atoms, the longest non-overlapping chain is 2 atoms, and 8 is more than twice 2, so this group is substantially linear.

Primarily Alkyl

Strictly speaking, the term alkyl refers to a monovalent radical obtained by removal of a hydrogen from an aliphatic hydrocarbon, and includes both saturated (alkanyl) and unsaturated (alkenyl, alkynyl) radicals However, it is customary in the art to use terms like “substituted alkyl”.

We have coined the term “primarily alkyl” to refer to an aliphatic moiety which is either an alkyl moiety in the strict sense of the term, or a moiety which differs from a strict alkyl moiety solely in that

(1) one or more hydrogens are replaced by halogen, hydroxyl, or sulfhydryl,

and/or

(2) there are a limited number of internal (thio)ether (C—O—C or C—S—C) linkages within the moiety.

The limitation imposed by (2) is that the ratio of the sum of the number of C—O—C and C—S—C linkages, to the number of C—C linkages, must be less than 1:5. However, note that even if a structure of the form X-Ch-Y does not qualify as a primarily alkyl moiety per se, the X and Y groups may still so qualify, the intervening -Ch- then qualifying as a spacer.

Like an alkyl group, a primarily alkyl group may have as little as a single carbon atom. However, it should be noted that in correlating a compound to a disclosed or claimed embodiment, it is desirable to interpret the features of the compound so as to minimize the number of “primarily alkyl moieties”. Thus —CH2-CH2(-CH2)-CH2 should be interpreted as a single primarily alkyl moiety, not as four or even as two primarily alkyl moieties.

Whenever a group is described as being “primarily alkyl”, the ratio stated above is preferably less than 1:10. More preferably, there are no internal (thio)ether linkages within the moiety. Preferably, a primarily alkyl group comprises at least one terminal moiety which is strongly lipophilic.

A “strictly alkyl” group is aliphatic and composed solely of hydrogen and carbon.

Primarily Alkanyl

A group is primarily alkanyl if (1) it is primarily alkyl, and (2) there are a limited number of C═C or C≡C linkages. The ratio of such linkages to the number of C—C must be less than 1:5. (Hence, a short primarily alkanyl group cannot contain any C═C or C≡C bonds.)

Whenever a group is described as being primarily alkanyl, the the ratio is preferably less than 1:10. More preferably, the moiety is strictly alkanyl. A “strictly alkanyl” group is a strictly alkyl group which is completely saturated.

Spacer Interpretation

In comparing a compound with a disclosed or claimed embodiment, there may be more than one way of correlating a spacer in a compound with a disclosed or claimed feature of the embodiment: (1) as a component of an expressly recited spacer cluster, e.g., in the recitation “-(spacer cluster)-primarily alkyl”; (2) as an expressly recited individual spacer, e.g., in the recitation “-(spacer)-primarily alkyl”; (3) as a component of a linker moiety, or other organic moiety, which as set forth expressly includes or can include a spacer; or (4) if —O— or —S—, as an implicitly allowed component of a primarily alkyl moiety. If so, then it is correlated in the aforestated order of preference, with (1) being the most preferred.

“Fatty” and “Fatty Acyl” Moieties

A fatty acid has the general structure R—C(═O)—OH, where R is a lipophilic organic moiety. The cognate “fatty acyl”moiety has the structure R—C(═O)—, where R is the same as for the original fatty acid. The cognate “fatty” moiety is the R of the original fatty acid and its cognate “fatty acyl” moiety.

Polyunsaturated Moiety

The compounds of the present invention may comprise at least one polyunsaturated moiety (PUM). This is defined as an aliphatic moiety comprising at least two alkenyl bonds (—C═C—). Preferably, there are two to ten alkenyl bonds. It is not required that any of the double bonds be of a cis, cis nature. However, that conformation is preferred.

Preferably, it is of the form —CH2-Rem or -spacer-Rem, where Rem is the remainder of the PUM. The —C(═O)-Rem structure is most preferred.

A PUM is not necessarily a primarily alkyl moiety, but it may be one. If it is not one, it is preferably of the form -spacer-unsaturated primarily alkyl.

The PUM is preferably substantially linear, more preferably linear. The PUM preferably consists only of carbon, hydrogen, and, optionally, nitrogen, oxygen and/or halogen, atoms. Preferably, it is composed of not more than 120 atoms other than hydrogen. More preferably, it is composed of not more than 90 such atoms, still more preferably not more than 60 such atoms, even more preferably not more than 40 such atoms, and most preferably not more than 30 such atoms.

The moiety may comprise at least one conjugated structure, that is, two immediately adjacent alkene moieties (—C═C—C═C—); at least one methylene-interrupted structure, that is, two alkene moieties separated by a single (unsubstituted or substituted) methylene (—C═C—C—C═C—); at least one polymethylene-interrupted structure, that is, two alkene moieties separated by two or more methylene units (—C═C—C—(C—)n C═C—, where n>1); or any combination of the foregoing. The methylene-interrupted structure is preferred.

The lipids of all plants and animals contain polyunsaturated fatty acids (PUFAs) with methylene-interrupted double bonds of the cis configurations. In higher plants, the number of double bonds rarely exceeds three, but in algae and animals there can be up to six. In nature, PUFAs are frequently derived either from linoleic (9-cis, 12-cis-octadecadienoic) or alpha-linolenic (9-cis, 12-cis,15-cis-octadecatrienoic) acids. In the shorthand lipid nomenclature these are 9c,12c-18:2 and 9c,12c,15c-18:3, respectively.

Another shorthand nomenclature used for methylene-interrupted PUFAs is the (n-x) form, where n denotes the chain length and x is the number of atoms from terminal double bond (the double bond furthest from the carbonyl carbon). This nomenclature is used only when all the double bonds are methylene-interrupted. In this nomenclature, linoleate and alpha-linolenate are n-6 and n-3 respectively. Preferably, the PUM is a methylene-interrupted “fatty” moiety, more preferably a “fatty acyl” moiety, belonging to one of the (n-6), (n-3), (n-9), (n-4), (n-1) and (n-7) families.

The n-6 family includes naturally occurring fatty acids of the forms 18:2(n-6), 18:3(n-6), 20:3(n-6), 20:4(n-6), 22:5(n-6) 20:2(n-6), 22:3(n-6), and 22:4(n-6). The most highly unsaturated naturally occurring fatty acid of the n-6 family is 28:7(n-6). Arachidonic acid, which is 20:4(n-6), is of particular interest.

The naturally occurring fatty acids of the n-3 family include 18:3(n-3), 20:3(n-3), 18:4(n-3), 20:4(n-3), 20:5(n-3), 22-:5(n-3), 22:6(n-3), 22:3(n-3), 16:3(n-3), 16:4(n-3), 18:5(n-3), 21:5(n-3), 24:5(n-3), 24:6(n-3), 38:7(n-3), 40:7(n-3), and, the most unsaturated member of the family, 28:8(n-3). The (n-9), (n-4), (n-1) and (n-7) families are also known to occur in nature.

For each of these fatty acids, there is a cognate “fatty” moiety. Preferably, the compounds of the present invention comprise a “fatty” moiety cognate to one of the foregoing naturally occurring forms, as this facilitates synthesis of the compound, and may also be beneficial in imparting particular biological activities to the compound.

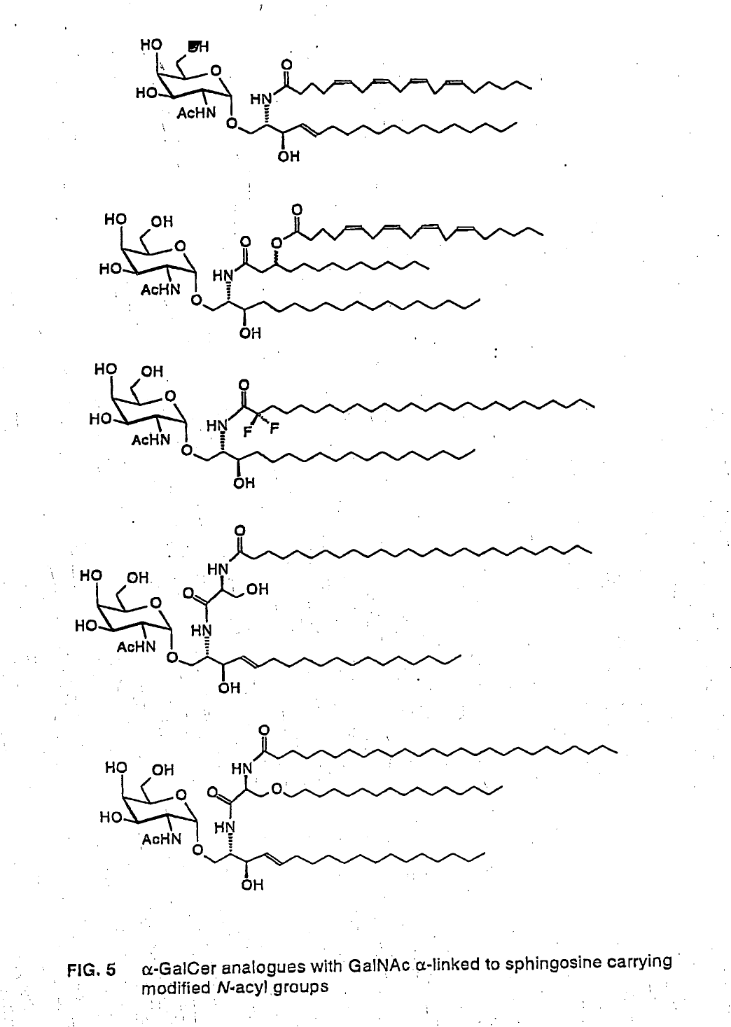

In FIG. 3, the third structure comprises a fatty acyl moiety which is indirectly connected to the nitrogen. This fatty acyl moiety is a methylene-interrupted fatty acyl moiety of the form 20:4(n-6), i.e., the same as arachidonic acid. The same fatty acyl moiety appears directly connected to the nitrogen, in the first structure of FIG. 5.

Alternatively, the PUM may comprise at least one conjugated pair of alkenic double bonds. Preferably, if the PUM comprises a conjugated system, it is a conjugated diene, triene, or tetraene, as such systems occur in naturally occurring fatty acids. Examples of naturally occurring conjugated fatty acids would be 2-trans,4-trans-hexadienoic (sorbic) acid, trans-10, trans-12-octadecadienoic acid, 9-cis, 11-trans,13-trans-octadecatrienoic acid, and 9-cis,11-trans,13-trans,15-cis-octadecateraenoic acid. Again, the PUM may comprise the corresponding “fatty” group.

Alternatively, the PUM may comprise at least one pair of polymethylene-interrupted alkenic double bonds. The term polymethylenic here denotes a chain of the form —C—(C—)n, where n>=1. The chain may be substituted or unsubstituted, the latter being preferred. Preferably, n=1, so that the alkenic carbons are separated by two alkanic carbons (ethylene-interrupted).

If the PUM comprises more than two alkenic double bonds, then combinations of the three basic types of paired systems (conjugated, methylene-interrupted, polymethylene-interrupted) are possible. For example, see pinolenic acid, which is 5-cis, 9-cis, 12-cis-octadecatrienoic acid, and therefore combines methylene-interrupted and ethylene-interrupted systems. Again, the PUM may comprise the corresponding “fatty” group.

Alkaloid Moiety

An alkaloid moiety is a moiety comprising one or more heterocyclic nitrogen atoms, which is not itself an amino acid, a peptide, a nucleotide, or a polynucleotide, and which does not comprise the cis-tetrahydro-2-oxothieno[3,4-d]imidazoline ring system of biotin (see below). A true alkaloid moiety is an alkaloid moiety which is derivable from an amino acid moiety precursor. A pseudoalkaloid moiety is an alkaloid moiety which is not derivable from an amino acid moiety precursor. A pseudoalkaloid moiety is derivable instead from a terpenoid or a purine moiety.

A biotinylated GalCer is known in the art. Since biotin, an imidazole derivative, comprises heterocyclic nitrogen, and it arguably can be synthesized from a benzyl-protected amino acid, see “Biotin: The Legacy http://www.scripps.edu/chem/baran/images/grpmtgpdf/Shenvi_Aug_{—}03.pdf, and especially Goldberg, U.S. Pat. No. 2,489,238, we believe it appropriate to expressly exclude it from our definition of an alkaloid moiety.

In a preferred embodiment, the alkaloid moiety does not comprise an imidazole ring.

In some embodiments, the alkaloid moiety is the residue of a alkaloid of plant origin, and in other embodiments, the alkaloid moiety is the residue of an alkaloid which is not of plant origin.

The ring system of an alkaloid may be one, two, three, four, five, size, or more-rings. The rings may be saturated or unsaturated, bridged or unbridged. Each ring may have three, four, five, six or more members. Two, three, four, five or more rings may be fused together. There may be one, two or more heterocyclic nitrogens, and these may be in the same or different rings. Also, they may be in fused or unfused rings.

One mode of classification of true alkaloids is on the basis of the potential AA precursor. Alkaloids are derivable from, inter alia, ornithine, lysine, phenylalanine, tyrosine and tryptophan. Cocaine and nicotine are derivable from Orn. The opiates thebaine, codeine and morphine are derivable from Phe or Tyr. Vinblastine and vincristine are derivable from Trp.

Another classification is as follows:

Pyridine group: piperine, coniine, trigonelline, arecaidine, guvacine, pilocarpine, cytisine, nicotine, sparteine

Pyrrolidine group: atropine, hyoscyamine, sparteine

Tropine group: atropine, cocaine, hygrine, ecgonine, pelletierine

Quinoline group: quinine, strychnine, brucine, veratrine, cevadine

Isoquinoline group: morphine, codeine, thebaine, papaverine, narcotine,narceine, hydrastine, berberine

Phenylethylamine group: methamphetamine, mescaline, ephedrine

Indole group: trypamine

Purine group: caffeine, theobromine, xanthine

glyoxaline: pilocarpine, ergotoxine, ergometrine

Residues of the foregoing alkaloids may be used as alkaloid moieties of the present invention, as may other alkaloids of the same or different groups.

It should be noted that both terpenoidal alkaloids and steroidal alkaloids are known in the art. Hence, the three classes (terpenoids, steroids, alkaloids) are not to be considered mutually exclusive.

The alkaloidal moieties of particular interest are those which are residues of alkaloids with immunomodulatory, antiviral, antimicrobial, antiparasitic or antitumor activity. Immunomodulatory alkaloids may be immunostimulatory, immunosuppressive, or both (on different immune functions, of course).

Immunosuppressive alkaloids include the indoles ibogaine and harmaline, and the bis-benzylisoquinoline tetrandine.

Immunostimulatory alkaloids include pentacyclic oxindole alkaloids from Cat's Claw (Uncaria tomentosa), manzamines from certain deep-sea Indo-Pacific sponges, swainsonine (8alphabeta-indolizidine-1alpha,2alpha,8beta-triol) and so forth.

Steroid Moiety

Steroids are compounds possessing the skeleton of cyclopenta[a]phenanthrene or a skeleton derived therefrom by one or more bond scissions or ring expansions or contractions. Methyl groups are normally present at C-10 and C-13. An alkyl side chain may also be present at C-17. Sterols are steroids containing a hydroxyl group at C-3 and most of the skeleton of cholestane. Additional carbon atoms may be present in the side chain.

A steroid moiety is the residue of a steroid as above defined.

Preferably, the steroid moiety has three 6-carbon rings and 1 5-carbon rings. Steroid moieties of interest include residues of testoterone, progesterone, cholesterol, stigmasterol, sitosterol, and the steroid moiety of compound BCI-054 (see table).

Terpenoid Moiety

Terpenes are compounds structurally related to isoprene. An isoprene unit is the carbon skeleton of isoprene, ignoring the double bonds. A terpene is a compound with a carbon skeleton consisting of one or more isoprene units. The branched end of the unit is considered the “head”, and the other end, the “tail”. The isoprene units may be joined head to tail, as in myrcine, tail to tail, as in squalene, or head to head. A hemiterpene is composed of one such unit (5 C atoms), a monoterpene is composed of two such units (hence 10 C atoms), a sesquiterpene of three units (15 C atoms), a diterpene of four units (20 C atoms), a sesterterpene of five units (25 C atoms), a triterpene of six units (30 C atoms), a tetraterprene of eight units (40 C atoms) and so forth. Alpha-phellandrene, methol and citral are monoterpenes. Alpha-selinene is a sesquiterpene. Myrcene, taxol (paclitaxel), docetaxol, and vitamin A are diterpenes. Squalene and bruceantin are triterpenes.

A terpenoid is a compound which, like a terpene, is structurally related to isoprene, but which may differ from strict additivity of isoprene units by the loss or shift of a fragment, normally a methyl group. The terpenoids therefore include the terpenes.

A terpenoid moiety is the residue of a terpenoid. The terpenoids of the present invention are preferably residues of monoterpenoids, sesquiterpenoids, diterpenoids, sesterterpenoids, triterpenoids, or tetraterpenoids.

The terpenoids of the present invention may be cyclic. Thus, they may be iridoids, which are cyclic monoterpenoids, having the iridane skeleton (1-isopropyl-2,3-dimethylcyclopentane). They may likewise be caratenoids, which are cyclized tetraterpenoids. Other cyclic terpenoids are included, too.

The terpenoids of the present invention may be hydrocarbons, or they may be substituted, e.g., with —OH or ═O.

It should be noted that some steroids are also terpenoids.

Lipophilic and Strongly Lipophilic Groups

Groups may be classified as lipophilic (hydrophobic), lipophobic (hydrophilic), or neutral. The lipophilicity of groups may be determined by measuring the partition coefficient of the molecule HZ (where Z is the side chain in question) between a nonpolar solvent (e.g., ethanol, dioxane, acetone, benzene, n-octanol) and water, at STP. The lipophilicity may be defined as the logarithm of this partition coefficient; it will then be positive for molecules which prefer the nonpolar solvent. Thus, a lipophilic group is one for which logP is greater than zero.

The partition coefficient (P) is defined as the ratio of the equilibrium concentrations of a dissolved substance in a two-phase system consisting of two largely immiscible solvents. One such system is n-octanol:water; the octanol phase will contain about 20% water and the water phase about 0.008% octanol. Thus, the relevant partition coefficient (Pow) is the ratio of the molar concentration of the solute in octanol saturated with water to its molar concentration in water saturated with octanol. N-octanol is a useful surrogate for biological membranes because it, like many membrane components, is amphiphilic. (Reference hereafter to log P shall mean log Pow, unless otherwise stated.)

For more information on methods of determining Pow, see Sangster, J., Octanol-Water Partition Coefficients: Fundamentals and Physical Chemistry (April 1997) (ISBN 0-471-9739).

For tabulations of octanol-water partition coefficients, see the EPA “Chemicals in the Environment: OPPT Chemicals Fact Sheets” the USDA Pesticide Properties Database, Sangster, J., “Octanol-Water Partition Coefficients of Simple Organic Compounds”, J. Phys. Chem. Ref. Data, 18:1111-1230 (1989); Verbruggen, E. M. J., et al., “Physiochemical Properties of Higher Nonaromatic Hydrocarbons: Literature Study,” J. Phys. Chem. Ref. Data, 29:1435-46 (2000). For more sources, see references cited at Penn State University Libraries, Physical Sciences Library, octanol-water Partition Coefficients (last updated Aug. 21, 2001), at the URL libraries.psu.edu/crsweb/physci/coefficients.htm. It should be noted that the Pow values compiled for different compounds may have been determined by different methodologies.

To avoid the need for experimental determinations of log Pow, for the purpose of the present invention, the value predicted by Meylan's method will be used.

In Meylan's method, the predicted log Pow is obtained by adding weighted coefficients for each fragment (the raw coefficient multiplied by the number of copies of that fragment) to the constant 0.2290. The fragments considered include

aliphatically attached —CH3 (0.5473), —CH2- (0.4911), —CH (0.3614), —OH (−1.4086), —NH2 (−1.4148), —C(═O)N (−0.5236), —SH (−0.0001), —NH— (−1.4962), —N═C (−0.0010), —O— (−1.2566), —CHO (−0.9422), -tert C so 3+ C attached (0.2676), C no H not tert (0.9723), —C(═O)O— (−0.9505), —C(═O)— (−1.5586), ═CH or C< (0.3836), #C (0.1334), —C(═O)N (−0.5236), —O—CO—C—N—CO (−0.5), —SO—O (−9), —O—P (−0.0162); O═P (−2.4239), phosphate attached —OH (0.475); aromatic C (0.2940), aromatic N (5 membered ring) (−0.5262), and aromatically attached —OH (−0.4802)

The Meylan algorithm is implemented in the program LogPow (KowWin). An online version of the program, available at esc.syrres.com/interkow/kowdemo.htm accepts either CAS registry numbers or SMILES structure notations. The program also reports experimentally determined values, if in its database.

A group is expected to be a lipophilic group if its logP, as predicted by the Meylan algorithm, is greater than zero.

For the purpose of this disclosure, a strongly lipophilic group is defined as being a group, comprising at least five atoms other than hydrogen, for which the predicted log P is at least 3.

Preferably, the logP predicted by the Meylan algorithm is at at least 4, at least 5, at least 6, at least 7, at least 8, at least 9, or at least 10, the higher the more preferred.

Preferably, the strongly lipophilic group will comprise not more than 100 atoms other than hydrogen, more preferably, not more than 80 such atoms, still more preferably, not more than 60 such atoms, even more preferably not more than 40 such atoms.

As noted previously, the strongly lipophilic group must comprise at least five atoms other than hydrogen. Preferably, it comprises at least six, more preferably at least 8, still more preferably at least 9, even preferably, it comprises at least 11 such atoms, still more preferably at least 13 such atoms, most preferably at least 21 such atoms.

Preferably, the strongly lipophilic group has an elemental composition limited to the elements carbon, silicon, hydrogen, oxygen, nitrogen, sulfur, and phosphorous. Preferably, the majority of the bonds within the side chain which do not involve hydrogen are carbon-carbon bonds.

Since the presence of oxygen, nitrogen, sulfur and phosphorous tends to reduce lipophilicity, in the strongly lipophilic group, preferably more than 50%, still more preferably more than 75%, of the non-hydrogen atoms are carbon atoms.

For the same reason, the strongly lipophilic group preferably comprises at least 5, at least 6, at least 7, at least 8, at least 9, or at least 10 carbon atoms.

Using the program LogKow, we have calculated (see below) low Pow values for certain structures:

| SMILES (lower case is arom) | Comments | PredLogP |
|---|---|---|
| CCCCC | alkyl (C5) | 2.8 |
| CCCCC C | alkyl (CE) | 3.29 |
| CCCCC CCCCC CCCCC CCCCC | alkyl (C20) | 10.16 |
| CCCC 0 CCCC | primarily | 3.01 |
| | alkanyl (CS) | |
| | with internal - | |
| | O- | |
| CC(C) (C)C | Pet Core | 2.69 |
| CCCCC CCCCC CCCC | alkyl (C14) | 7.22 |
| O═C CCCCC CCCCC CCC | acyl (14:0) | 5.73 |
| CO CC(O) CCCCC CCCCC C | acyl 14:0, 3-OH | 4.19 |
| O═C CC(═O) CCCCC CCCCC | acyl 13:0 with | 3.68 |
| | internal | |
| | carbonyl | |

The predicted logP is used even if an experimental logP is available, e.g., for Pet core, it is 3.11.

Carbon Chains

The strongly lipophilic group will in general comprise one or more carbon chains. Each carbon chain will be composed of carbon atoms linked sequentially by single, double or triple bonds.

Carbon chains which are at least sik carbons in length are considered “major” carbon chains. Other carbon chain are considered “minor” carbon chains. The strongly lipophilic group preferably comprises at least one major carbon chain. There is no preference one way or another as to the presence of minor carbon chains.

Minor carbon chains can be considered a species of linker.

The carbon atoms of a carbon chain may be bonded to 3, 2, 1 or 0 hydrogens. In a major carbon chain, the —CH< and >C< carbons are usually branching points for the attachment (with or without a linker) of another carbon chain. They may also be substituted with a side group, such as amino or hydroxyl.

Purely as a matter of definition, the strongly lipophilic group cannot comprise a Pet unit (it may comprise a Pet core if it lacks one or more of the required spacers Y1-Y4) However, what might otherwise have been interpreted as one large strongly lipophilic group comprising a Pet unit may be reinterpreted as a Pet unit with one or more smaller strongly lipophilic groups attached to it.

The carbon atoms of any major carbon chain may include one or more carbonyl or thiocarbonyl carbons, i.e., —C(═O)— or —C(═S)—. Carbonyl is preferred. If there is only one carbonyl or thiocarbonyl carbon, it is preferably at the beginning of the chain, so the chain is an acyl chain (saturated or unsaturated). Thus, if the linker is —O—, the attachment to carbonyl forms an ester (—O—(C═O)—), and if it is —NH—, the attachment forms an amide (—NH—(C═O)—.

A particular lipophilic group may be a simple (unbranched, acyclic) lipid, or a complex (branched and/or cyclic, including partially aromatic) lipid.

If the lipophilic group comprises more than one major carbon chain, the major chain beginning closest to the sugar or pet core is considered the primary major chain of the group. Any chains attached to the primary major chain are considered secondary major chains. Any major chains attached to the secondary major chains are considered tertiary major chains, etc. (Reference to primary, secondary, etc. chains hereafter is to major chains unless otherwise indicated.)

It is possible that several major chains will be equally close to the sugar or Pet core, in which case they will each be primary chains.

A secondary chain may be attached to the distal end (relative to the sugar or Pet core) of the primary chain, in which case the lipophilic group remains linear (absent other moieties). Or it may be attached to an interior carbon of the primary chain, in which case the lipophilic group is a branched lipid.

A secondary chain may be attached to a primary chain by a simple —O—, —S— or —NH— linker, or it may be attached directly without a linker (i.e., C—C). It also may be attached by a complex linker, i.e., a combination of a simple linker and the distal linker previously defined. A tertiary chain may be attached to a secondary chain in the same manner, and so on. A preferred point of attachment of a higher order chain to a lower order chain (e.g. secondary to primary) is at the C-3 carbon of the lower order (e.g., primary) chain.

Like a primary chain, a secondary or higher order chain may comprise doubly or triply bonded carbon atoms, and/or carbonyl or thiocarbonyl carbons.

The various carbon chains referred to above may be substituted with hydroxyl or amino groups, with hydroxyl being preferred. Preferred positions for the hydroxyl group would be as substituents on the C-2 or C-3 carbon of the chain.

The strongly lipophilic group may be entirely aliphatic or (unless expressly excluded by another limitation) it may be partially aromatic in character. If it includes an aromatic structure, that structure is deemed a separate major carbon chain even if directly attached to an aliphatic chain.

Non-Naturally Occurring

When a compound is identified as non-naturally occurring, that means only that it does not occur as the result of wholly natural processes. If an organism is genetically engineered to produce a compound that otherwise would not be produced in a biological system, then the organism is not wholly natural, and its production of the compound does not make the compound a naturally occurring one.

Also, just because a compound is identified as non-naturally occurring does not exclude the possibilities that (1) it exists in nature as a portion of a larger, naturally occurring compound, (2) portions of the non-naturally occurring compound occur, as compounds in their own right, in nature, or (3) portions of the naturally occurring compound occur as parts of other, naturally occurring, compounds.

Phosphate Equivalents

The present disclosure contains a proviso excluding, from certain Pet unit-containing compounds, certain phosphate equivalents that were featured in previously disclosed lipid A analogues.

The following moieties are considered phosphate equivalents: —O—P(═O)(OH)—O—, —C(═O)OH, —O—S(═O)₂—O—, or —O—B(OH)—O— moiety, these being listed in order from most to least preferred. Note that this list includes phosphate itself.

Analogues and Homologues

Also of interest are analogues of the disclosed compounds which are identified on the basis of structural similarity as determined by “fingerprinting” software.

Analogues may be identified by assigning a hashed bitmap structural fingerprint to the compound, based on its chemical structure, and determining the similarity of that fingerprint to that of each compound in a broad chemical database. The fingerprints are determined by the fingerprinting software commercially distributed for that purpose by Daylight Chemical Information Systems, Inc., according to the software release current as of Jan. 8, 1999. In essence, this algorithm generates a bit pattern for each atom, and for its nearest neighbors, with paths up to 7 bonds long. Each pattern serves as a seed to a pseudorandom number generator, the output of which is a set of bits which is logically OR-ed to the developing fingerprint. The fingerprint may be fixed or variable size.

The database may be SPRESI'95 (InfoChem GmbH), Index Chemicus (ISI), MedChem (Pomona/Biobyte), World Drug Index (Derwent), TSCA93(EPA) Maybridge organic chemical catalog (Maybridge), Available Chemicals Directory (MDLIS Inc.), NCI96 (NCI), Asinex catalog of organic compounds (Asinex Ltd.), or IBIOScreen SC and NP (Inter BioScreen Ltd.), or an inhouse database.

A compound is an analogue of a reference compound if it has a Daylight fingerprint with a similarity (Tanamoto coefficient) of at least 0.85 to the Daylight fingerprint of the reference compound.

A compound is also an analogue of a reference compound if it may be conceptually derived from the reference compound by isosteric replacements or homologous changes.

Homologues are compounds which differ by an increase or decrease in the number of methylene groups in an alkyl moiety.

Classical isosteres are those which meet Erlenmeyer's definition: “atoms, ions or molecules in which the peripheral layers of electrons can be considered to be identical”. Classical isosteres include

| Monovalents | Bivalents | Trivalents | Tetra | Annular |
|---|---|---|---|---|
| F, OH, NH₂, CH₃ | —O— | —N═ | ═C═ | —CH═CH— |
| | | | ═Si═ | |
| C1, SH, PH₂ | —S— | —P═ | —N+═ | —S— |
| Br | —Se— | —As— | ═P+═ | —O— |
| i | —Te— | —Sb— | ═As+═ | —NH— |
| | | | —CH═ | ═Sb+═ |

Nonclassical isosteric pairs include —CO— and —SO₂—, —COOH and —SO₃H, —SO₂NH₂ and —PO(OH)NH₂, —H and —F, —OC(═O)— and C(═O)O—, and —OH and —NH₂.

Compositions

A composition of the present invention comprises at least one compound of the present invention, as previously described, in a therapeutically effective amount.

When said compound is immunostimulatory, the composition may further comprise at least one immunogen.

The composition may comprise, with or without said immunogen, at least one other immunostimulatory agent (adjuvant), such as a lipid-A derivative, CpG containing oligonucleotide, Muramyl di-peptide, sitosterol, alum, QS-21 or any other adjuvant preparation that stimulates the immune system.

Combinations

Any of the compounds of the present invention may be used in combination with each other, with other immunological agents, and with other pharmaceutical agents. Immunological agents include antigens (including both immunogens and haptens), adjuvants, and other immodulatory molecules (including cytokines).

A combination may be a covalent conjugate, a noncovalent conjugate, a simple mixture, or use such that all of the elements of the combination are simultaneously active in the subject to which they are administered. Simultaneous activity may, but need not, be achieved by'simultaneous administration. Compounds may be simultaneously active even if they are not simultaneously administered, e.g, compound A with a long half-life is administered prior to compound B with a short half-life, but A is still present in the body at an effective level when B is administered.

Immunogen

The immunogen of the present invention is a molecule, comprising at least one disease-associated B or T cell epitope, as defined below, and which, when suitably administered to a subject (which, in some cases, may mean associated with a liposome or with an antigen-presenting cell), elicits a humoral and/or cellular immune response which is protective against the disease.

The present invention contemplates, in some embodiments, the use of the disclosed compounds

(1) to stimulate innate immunity, and/or

(2) to adjuvant the specific immune response to an administered immunogen.

If the epitope is a carbohydrate epitope, it may be an analog of a naturally occurring epitope containing at least one amino sugar, in which at least one amino sugar is replaced with an aminated Pet unit.

Epitope

The epitopes of the present invention may be B-cell or T-cell epitopes, and they may be of any chemical nature, including without limitation peptides, carbohydrates, lipids, glycopeptides and glycolipids. The epitope may be identical to a naturally occurring epitope, or a modified form of a naturally occurring epitope.

A term such as “MUC1 epitope”, without further qualification, is intended to encompass, not only a native epitope of MUC1, but also a mutant epitope which is substantially identical to a native epitope. Such a mutant epitope must be cross-reactive with a native MUC1 epitope. Likewise, a term such as “tumor-associated epitope” includes both native and mutant epitopes, but the mutant epitope must be cross-reactive with a native tumor-associated epitope.

B-cell Epitopes

B-cell epitopes are epitopes recognized by B-cells and by antibodies. B-cell peptide epitopes are typically at least five amino acids, more often at least six amino acids, still more often at least seven or eight amino acids in length, and may, be continuous (“linear”) or discontinuous (“conformational”) (the latter being formed by the folding of a protein to bring noncontiguous parts of the primary amino acid sequence into physical proximity). B-cell epitopes may also be carbohydrate epitopes.

T-cell Epitopes

The T cell epitope, if any, may be any T cell epitope which is at least substantially the same as a T-cell epitope of an antigen including a hapten) which is associated with a disease or adverse condition to a degree such that it could be prophylactically or therapeutically useful to stimulate or enhance a cellular immune response to that epitope. Such diseases and conditions include, but are not limited to parasitic diseases such as schistosomiasis and leishmania, fungal infections such as candidiasis, bacterial infections such as leprosy, viral infections such as HIV infections, and cancers, especially solid tumors. Of course, the greater the degree of specificity of the epitope for the associated disease or adverse condition, the more likely it is that the stimulation of an immune response to that epitope will be free of adverse effects.

The epitope must, of course, be one amenable to recognition by T-cell receptors so that a cellular immune response can occur. For peptides, the T-cell epitopes may interact with class I or class II MHC molecules. The class I epitopes usually, 8 to 15, more often 9-11 amino acids in length. The class II epitopes are usually 5-24 (a 24 mer is the longest peptide which can fit in the Class II groove), more often 8-24 amino acids. If the immunogen is larger than these sizes, it will be processed by the immune system into fragments of a size more suitable for interaction with MHC class I or II molecules.

The carbohydrate T-cell epitopes may be as small as a single sugar unit (e.g., Tn). They are preferably no larger than five sugars.

Many T-cell epitopes are known. Several techniques of identifying additional. T-cell epitopes are recognized by the art. In general, these involve preparing a molecule which potentially provides a T-cell epitope and characterizing the immune response to that molecule. Methods of characterizing the immune response are discussed in a later section.

The reference to a CTL epitope as being “restricted” by a particular allele of MHC Class I molecules, such as HLA-A1, indicates that such epitope is bound and presented by the allelic form in question. It does not mean that said epitope might not also be bound and presented by a different allelic form of MHC, such as HLA-A2, HLA-A3, HLA-B7, or HLA-B44.

Disease-Associated and Disease-Specific Epitopes

A disease is an adverse clinical condition caused by infection or parasitization by a virus, unicellular organism, or multicellular organism, or by the development or proliferation of cancer (tumor) cells.

The unicellular organism may be any unicellular pathogen or parasite, including a bacteria, fungus or protozoan. The multicellular organism may be any pathogen or parasite, including a protozoan, worm, or arthropod. Multicellular organisms include both endoparasites and ectoparasites. Endoparasites are more likely to elicit an immune, response, but, to the extent they can elicit a protective immune response, ectoparasites and their antigens are within the purview of the present invention.

An epitope may be said to be directly associated with a viral disease if it is presented by a virus particle, or if it is encoded by the viral genome and expressed in an infected cell.

An epitope may be said to be directly associated with a disease caused by a unicellular or multicellular organism if it presented by an intracellular, surface, or secreted antigen of the causative organism.

An epitope may be said to be directly associated with a particular tumor if it is presented by an intracellular, surface or secreted antigen of said tumor. It need not be presented by all cell lines of the tumor type in question, or by all cells of a particular tumor, or throughout the entire life of the tumor. It need not be specific to the tumor in question. An epitope may be said to be “tumor associated” in general if it is so associated with any tumor (cancer, neoplasm).

Tumors may be of mesenchymal or epithelial origin. Cancers include cancers of the colon, rectum, cervix, breast, lung, stomach, uterus, skin, mouth, tung, lips, larynx, kidney, bladder, prostate, brain, and blood cells.

An epitope may,be indirectly associated with a disease if the epitope is of an antigen which is specifically produced or overproduced by infected cells of the subject, or which is specifically produced or overproduced by other cells of the subject in specific, but non-immunological, response to the disease, e.g., an angiogenic factor which is overexpressed by nearby cells as a result of regulatory substances secreted by a tumor.

The term “disease associated epitope” also includes any non-naturally occurring epitope which is sufficiently similar to an epitope naturally associated with the disease in question so that antibodies or T cells which recognize the natural disease epitope also recognize the similar non-natural epitope. Similar comments apply to epitopes associated with particular diseases or classes of diseases.

An epitope may be said to be specific to a particular source (such as a disease-causing organism, or, more particular, a tumor), if it is associated more frequently with that source than with other sources, to a detectable and clinically useful extent. Absolute specificity is not required, provided that a useful prophylactic, therapeutic or diagnostic effect is still obtained.

In the case of a “specific tumor-specific” epitope, the epitope is more frequently associated with that tumor that with other tumors, or with normal cells. Preferably, there should be a statistically significant (p=0.05) difference between its frequency of occurrence in association with the tumor in question, and its frequency of occurrence in association with (a) normal cells of the type from which the tumor is derived, and (b) at least one other type of tumor. An epitope may be said to be “tumor-specific” in general is it is associated more frequently with tumors (of any or all types) than with normal cells. It need not be associated with all tumors.

The term “tumor specific epitope” also includes any non-naturally occurring epitope which is sufficiently similar to a naturally occurring epitope specific to the tumor in question (or as appropriate, specific to tumors in general) so that antibodies or T cells stimulated by the similar epitope will be essentially as specific as CTLs stimulated by the natural epitope.

In general, tumor-versus-normal specificity is more important than tumor-versus-tumor specificity as (depending on the route of administration and the particular normal tissue affected), higher specificity generally leads to fewer adverse effects. Tumor-versus-tumor specificity is more important in diagnostic as opposed to therapeutic uses.

The term “specific” is not intended to connote absolute specificity, merely a clinically useful difference in probability of occurrence in association with a pathogen or tumor rather than in a matched normal subject.

In one embodiment, the epitope is a parasite-associated epitope, such as an epitope associated with leishmania, malaria, trypanosomiasis, babesiosis, or schistosomiasis. In another embodiment, the epitope is a viral epitope, such as an epitope associated with human immunodeficiency virus (HIV), Epstein-Barr virus (EBV), or hepatitis.

The epitope may also be associated with a bacterial antigen, such as an antigen of the tuberculosis bacterium, Staphylococcus, E. coli or Shigella sonnei.

In another embodiment, the epitope is associated with a cancer (tumor), including but not limited to cancers of the respiratory system (lung, trachea, larynx), digestive system (mouth, throat, stomach, intestines) excretory system (kidney, bladder, colon, rectum), nervous system (brain), reproductive system (ovary, uterus, cervix), glandular system (breast, liver, pancreas, prostate), skin, etc. The two main groups of cancers are sarcomas, which are of mesenchymal origin and affect such tissues as bones end muscles, and carcinomas, which are of epithelial origin and make up the great majority of the glandular cancers of breasts, stomach, uterus, skin and tongue. The sarcomas include fibrosarcomas, lymphosarcomas, osteosarcomas, chondrosarcomas, rhabdosarcomas and liposarcomas. The carcinomas include adenocarcinomas, basal cell carcinomas and squamous carcinomas.

Cancer-associated: epitopes include, but are not limited to, peptide epitopes such as those, of mutant p53, the point mutated Ras oncogene gene product, her 2/neu, c/erb2, and the MUC1 core protein, and carbohydrate epitopes such as sialyl Tn (STn), TF, Tn, CA 125, sialyl Le^{x}, sialyl Le^{a} and P97.

Identification of Natural Epitopes

Naturally occurring epitopes may be identified by a divide-and-test process. One starts with a protein known to be antigenic or immunogenic. One next tests fragments of the protein for immunological activity. These fragments may be obtained by treatment of the protein with a proteolytic agent, or, if the peptide sequence is known, one may synthetically prepare smaller peptides corresponding to subsequences of the protein. The tested fragments may span the entire protein sequence, or just a portion thereof, and they may be abutting, overlapping, or separated.

If any of the fragments are immunologically active, the active fragments may themselves be subjected to a divide-and-test analysis, and the process may, be continued until the minimal length immunologically active sequences are identified. This approach may be used to identify either B-cell or T-cell-epitopes, although the assays will of course be different. Geysen teaches systematically screening all possible oligopepitide (pref. 6-10 a.a.) abutting or overlapping fragments of a particular protein for immunological activity in, order to identify linear epitopes. See WO 84/03564.

It is also possible to predict the location of B-cell or T-cell peptide epitopes if an amino acid sequence is available. B-cell epitopes tend to be in regions of high local average hydrophilicity. See Hopp and Wood, Proc. Nat. Acad. Sci. (USA) 78: 3824 (1981); Jameson and Wolf, CABIOS, 4: 181 (1988). T-cell epitopes can be predicted, on the basis of known consensus sequences for the peptides bound to MHC class I molecules of cells of a particular haplotype. See e.g., Slingluff, WO98/33810, especially pp. 15-16; Parker, et al., “Scheme for ranking potential HLA-A2 binding peptides based on independent binding of individual peptide side chains”, J. Immunol. 152: 163 (1994).

Naturally occurring T-cell epitopes may be recovered by dissociating them from their complexes with MHC class I molecules and then sequencing them, e.g., by mass spectroscopic techniques.

Generally speaking, in addition to epitopes which are identical to the naturally occurring disease- or tumor-specific epitopes, the present invention embraces epitopes which are different from but substantially identical with such epitopes, and therefore disease- or tumor-specific in their own right. It also includes epitopes which are not substantial identical to a naturally occurring epitope, but which are nonetheless cross-reactive with the latter as a result of a similarity in 3D conformation.

Peptide Epitopes

A peptide epitope is considered substantially identical to a reference peptide epitope (e.g., a naturally occurring epitope) if it has at least 10% of an immunological activity of the reference epitope and differs from the reference epitope by no more than one non-conservative substitution.

Carbohydrate Haptens; Epitopes

The carbohydrate, hapten of the present invention is a carbohydrate which comprises (and preferably is identical to) a carbohydrate epitope, but which does not elicit a humoral immune response by itself.

Normally, a carbohydrate hapten will not be a polysaccharide, as a polysaccharide is usually large enough to be immunogenic in its own right. The borderline between an oligosaccharide and a polysaccharide is not fixed, however, we will define an oligosaccharide as consisting of 2 to 20 monosaccharide (sugar) units.

The hapten may be a monosaccharide (without glyosidic connection to another such unit), or an oligosaccharide. If an oligosaccharide, it preferably is not more than 10 sugar units.

Tumor associated carbohydrate epitopes are of particular interest.

A variety of carbohydrates can be conjugated according to the present invention, for use particularly in detecting and treating tumors. The Tn, T, sialyl Tn and sialyl (2→6)T haptens are particularly preferred.

In particular, for detecting and treating tumors, the three types of tumor-associated carbohydrate epitopes which are highly expressed in common human cancers are conjugated to aminated compounds. These particularly include the lacto series type 1 and type 2 chain, cancer associated ganglio chains, and neutral-glycosphingolipids.

Examples of the lacto series Type 1 and Type 2 chains are as follows: Lewis a, dimeric Lewis a, Lewis b, Lewis b/Lewis a, Lewis x, Lewis, y, Lewis a/Lewis x. dimeric Lewis x, Lewis y/Lewis x, trifucosyl Lewis y, trifucosyl Lewis b, Sialosyl Lewis x, sialosyl Lewis y, sialosyl dimeric Lewis x, Tn, sialosyl Tn, sialosyl TF, TF. Examples of cancer-associated ganglio chains are as follows: GM3. GD3, GM2, GM4, GD2, GM1, GD-1a, GD-1b. Neutral sphingolipids include globotriose, globotetraose, globopentaose, isoglobotriose, isoglobotetraose, mucotriose, mucotetraose, lactotriose, lactotetraose, neolactotetraose, gangliotriose, gangliotetraose, galabiose, and 9-O-acetyl-GD3.

Numerous antigens of clinical significance bear carbohydrate determinants. One group of such antigens comprises the tumor-associated mucins (Roussel, et al., Biochimie 70, 1471, 1988).

Generally, mucins are glycoproteins found in saliva, gastric juices, etc., that form viscous solutions and act as lubricants or protectants on external and internal surfaces of the body. Mucins are of high molecular weight (often >1,000,000 Dalton) and extensively glycosylated. The glycan chains of mucins are O-linked (to serine or threonine residues) and may amount to more than 80% of the molecular mass of the glycoprotein. Mucins are produced by ductal epithelial cells and by tumors of the same origin, and may be secreted, or cell-bound as integral membrane proteins (Burchell, et al., Cancer Res., 47, 5476, 1987; Jerome, et al., Cancer Res., 51, 2908, 1991).

Cancerous tissues produce aberrant mucins which are known to be relatively less glycosylated than their normal counter parts (Hull et al., Cancer Commun., 1, 261, 1989). Due to functional alterations of the protein glycosylation machinery in cancer cells, tumor-associated mucins typically contain short, incomplete glycans. Thus, while the normal mucin associated with human milk fat globules consists primarily of the tetrasaccharide glycan, gal β1-4 glcNAcp1-6(gal β1-3) gal NAc-α and its sialylated analogs (Hull, et al.), the tumor-associated Tn hapten consists only of the monosaccharide residue; α-2-acetamido-3-deoxy-D-galactopyranosyl, and the T-hapten of the disaccharide β-D-galactopyranosyl-(1-3)α-acetamido-2-deoxy-D-galactopyranosyl. Other haptens of tumor-associated mucins, such as the sialyl-Tn and the sialyl-7(2-6)T haptens,, arise from the attachment of terminal sialyl residues to, the short Tn and T glycans (Hanisch, et al., Biol. Chem. Hoppe-Seyler, 370,. 21, 1989; Hakormori, Adv. Cancer Res., 52:257, 1989; Torben, et al., Int. J. Cancer, 45 666, 1980; Samuel, et al., Cancer Res., 50, 4801, 1990).

The T and Tn antigens (Springer, Science, 224, 1198, 1984) are found in immunoreactive form on the external surface membranes of most primary carcinoma cells and their metastases (>90% of all human carcinomas). As cancer markers, T and Tn permit early immunohistochemical detection and prognostication of the invasiveness of some carcinomas (Springer). Recently, the presence of the sialyl-Tn hapten on tumor tissue has been identified as an unfavorable prognostic parameter (Itzkowitz, et al. Cancer, 66, 1960, 1990; Yonezawa, et al., Am. J. Clin. Pathol., 98 167, 1992). Three different types of tumor-associated carbohydrate antigens are highly expressed in common human cancers. The T and Tn haptens are included in the lacto series type, and type 2 chains. Additionally, cancer-associated ganglio chains and glycosphingolipids are expressed on a variety of human cancers.

The altered glycan determinants displayed by the cancer associated mucins are recognized as non-self or foreign by the patient's immune system (Springer). Indeed, in most patients, a strong autoimmune response to the T hapten is observed. These responses can readily be measured, and they permit the detection of carcinomas with greater sensitivity and specificity, earlier than has previously been possible. Finally, the extent of expression of T and Tn often correlates with the degree of differentiation of carcinomas. (Springer).

An extensive discussion of carbohydrate haptens appears in Wong, U.S. Pat. No. 6,013,779. A variety of carbohydrates can be incorporated into a synthetic glycolipopeptide immunogen, according to the present invention, for use particularly in detecting and treating tumors. The Tn, T, sialyl Tn and sialyl (2→6)T haptens are particularly preferred.

In particular, for detecting and treating tumors, the three types of tumor-associated carbohydrate epitopes which are highly expressed in common human cancers are conjugated to aminated compounds. These particularly include the lacto series type 1 and type 2 chain, cancer associated ganglio chains, and neutral glycosphingolipids.

Examples of the lacto-series Type 1 and Type 2 chains are as, follows:

Lacto Series Type 1 and Type 2 Chains

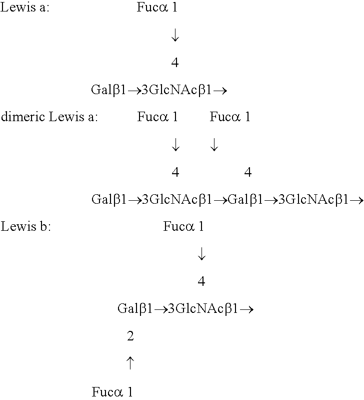

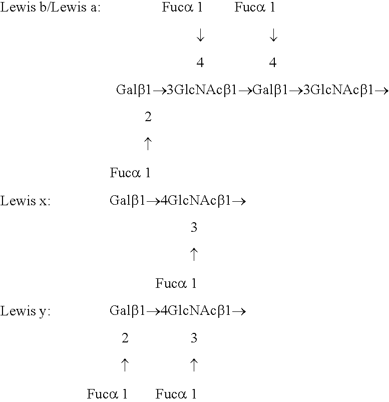

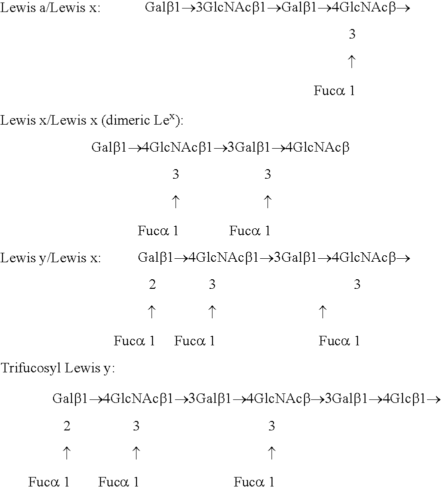

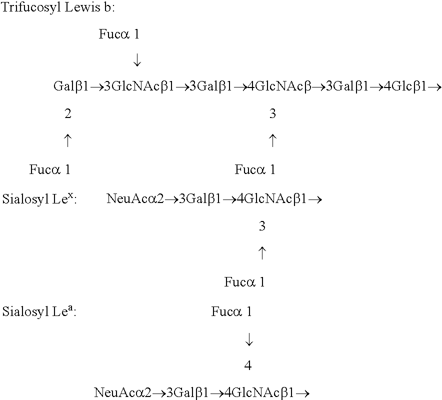

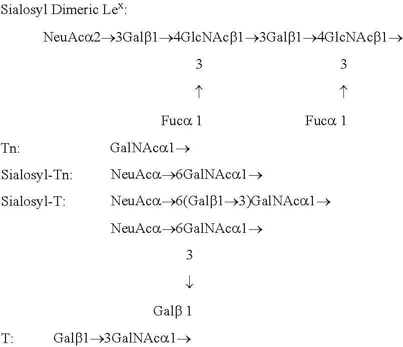

Examples of cancer-associated ganglio chains that can be conjugated to aminated compounds according to; the present invention are as follows:

Cancer Associated Ganglio Chains

CANCER ASSOCIATED GANGLIO CHAINS

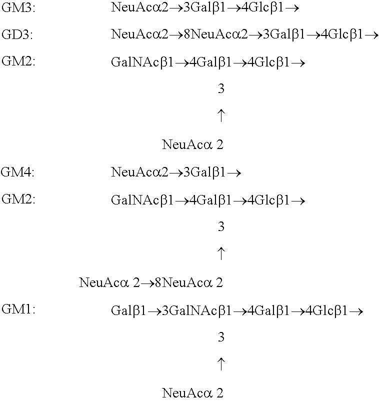

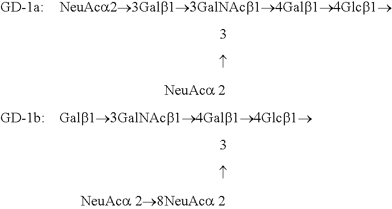

In addition to the above, neutral glycosphingolipids can also be conjugated to aminated compounds according to the present invention:

Selected Neutral Glycosphingolipids

| | |
|---|---|
| Globotriose: | Galα→4Galβ1→4Glcβ1→ |
| Globotetraose: | GalNAcβ1→3Galα→4Galβ1→4Glcβ1→ |
| Globopentaose: | GalNAcα1→3GalNAcβ1→3Galα→4Galβ→ |
| | 4Glcβ1→ |
| Isoglobotriose: | Galα→3Galβ1→4Glcβ1→ |
| Isoglobotetraose: | GalNAcβ1→3Galα1→3Galβ1→4Glcβ1→ |
| Mucotriose: | Galβ1→4Galβ1→4Glcβ1→ |
| Mucotetraose: | Galβ1→3Galβ1→4Galβ1→4Glcβ1→ |
| Lactotriose: | GalNAcβ1→3Galβ1→4Glcβ1→ |
| Lactotetraose: | GalNAcβ1→3GalNAcβ1→3Galβ1→4Glcβ1→ |
| Neolactotetraose: | Galβ1→4GlcNAcβ1→3Galβ1→4Glcβ1→ |
| Gangliotriose: | GalNAcβ1→4Galβ1→4Glcβ1→ |
| Gangliotetraose: | Galβ1→GlcNAcβ1→4Galβ1→4Glcβ1→ |
| Galabiose: | Galα→4Galβ1→ |
| 9-O-Acetyl-GD3: | 9-O-Ac-NeuAcα2→8NeuAcα2→3Galβ1→4Glcβ1→ |

Immunoconjugates

The immunogen of the present invention may be an immunoconjugate in which one or more epitopes are joined with other chemical moieties to create a molecule with different immunological properties, such as increased ability to elicit a humoral immune response. For example, one or more epitopes may be conjugated to a macromolecular carrier, such as albumin, keyhole limpet hhemocyanin (KLH) or polydextran. Or several epitopes may be joined to a branched lysine core, such as a MAP-4 peptide. Or several epitopes may simply be conjugated together using some other linker or molecular scaffold.

Adjuvants

It is generally understood, that a synthetic antigen of low molecular weight can be weakly immunogenic, which is the biggest obstacle to the success of a fully synthetic vaccine. One way to improve the imunogenicity of such a synthetic antigen is to deliver it in the environment of an adjuvant.

As conventionally known in the art, adjuvants are substances that act in conjunction with specific antigenic stimuli to enhance the specific response to the antigen. An ideal adjuvant is believed to non-specifically stimulate the immune system of the host, which upon the subsequent encounter of any foreign antigen can produce strong and specific immune response to that foreign antigen. Such strong and specific immune response, which is also characterized by its memory, can be produced only when T-lymphocytes (T-cells) of the host immune system are activated.

T-cell blastogenesis and IFN-gamma production are two important parameters for measuring the immune response. Experimentally, T-cell blastogenesis measures. DNA synthesis that directly relates to T-cell proliferation, which in turn is the direct result of the T-cell activation. On the other hand, IFN-gamma is a major cytokine secreted by T-cells when they are activated. Therefore, both T-cell blastogenesis and IFN-gamma production indicate T-cell activation, which suggests the ability of an adjuvant in helping the host immune system to induce a strong and specific immune response to any protein-based antigen.

The compound is considered an adjuvant if it significantly (p=0.05) increases the level of either T-cell blastogenesis or of interferon gamma production in response to at least one liposome/immunogen combination relative to the level elicited by the immunogen alone. Preferably, it does both. Preferably, the increase is at least 10% , more preferably at least 50%, still more preferably, at least 100%.

Preferably, the toxicity of the lipid compounds of the present invention is not more than 50% that of said natural Lipid-A product; more preferably it is less than 10% that of the latter.

A large number of adjuvants are known in the art, including Freund's complete adjuvant, saponin, DETOX (Ribi Immunochemicals), Montanide ISA-51, -50 and -70, QS-21, monophosphoryl lipid A and analogs thereof. A lipid adjuvant can be presented in the context of a liposome.

The present liposomal vaccines may be formulated advantageously with an adjuvant. Monophosphoryl lipid A (MPLA), for example, is an effective adjuvant that causes increased presentation of liposomal antigen to specific T Lymphocytes. Alving, C. R., Immunobiol., 187: 430-446 (1993). The skilled artisan will recognize that lipid-based adjuvants, such as Lipid A and derivatives thereof, are also suitable. A muramyl dipeptide (MDP), when incorporated into liposomes, has also been shown to increase adjuvanticity (Gupta R K et al., Adjuvants-A balance between toxicity and adjuvanticity,” Vaccine, 11, 293-306 (1993)).

Use of an adjuvant is not required for immunization.

Liposome Formulations

Liposomes are microscopic vesicles that consist of one or more lipid bilayers surrounding aqueous compartments. See e.g., Bakker-Woudenberg et al., Eur. J. Clin. Microbiol. Infect. Dis. 12 (Suppl.1): S61 (1993) and Kim, Drugs; 46: 618 (1993). Because liposomes can be formulated with bulk lipid molecules that are also found in natural cellular membranes, liposomes generally can be administered safely and are biodegradable.

Liposomes are globular particles formed by the physical self-assembly of polar lipids, which define the membrane organization in liposomes. Liposomes may be formed as uni-lamellar or multi-lamellar vesicles of various sizes. Such liposomes, though, constituted of small molecules having no immunogenic properties of their own, behave like macromollecular particles and display strong immunogenic characteristics.

Depending on the method of preparation, liposomes may be unilamellar or multilamellar, and can vary in size with diameters ranging from about 0.02 microm to greater than about 10 microm. A variety of agents can be encapsulated in liposomes. Hydrophobic agents partition in the bilayers and hydrophilic agents partition within the inner aqueous space(s). See e.g., Machy et al., Liposomes in Cell Biology and Pharmacology (John Libbey, 1987), and Ostro et al., American J. Hosp. Pharm. 46: 1576 (1989).

Liposomes can adsorb to virtually any type of cell and then release an incorporated agent. Alternatively, the liposome can fuse with the target cell, whereby the contents of the liposome empty into the target cell. Alternatively, a liposome may be endocytosed by cells that are phagocytic. Endocytosis is followed by intralysosomal degradation of liposomal lipids and release of the encapsulated agents. Scherphof et al., Ann. N.Y. Acad. Sci., 446: 368 (1985).

Other suitable liposomes that are used in the methods of the invention include multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV). The skilled artisan will recognize that the techniques for preparing these liposomes are well known in the art. See Colloidal Drug, Delivery Systems, vol. 66 (J. Kreuter, ed., Marcel Dekker, Inc., 1994).

A “liposomal formulation” is an in vitro-created lipid vesicle in which a pharmaceutical agent, such as an antigen, of the present invention can be incorporated or to which one can be attached. Thus, “liposomally bound” refers to an agent that is partially incorporated in or attached to a liposome. The immunogen of the present invention may be a liposomally-bound antigen which, but for said liposome, would not be an immunogen, or it may be immunogenic, even in a liposome-free state. Several different agents may be incorporated into or attached to the same liposome, or different agents may be associated with different liposomes, and the liposomes administered separately or together to a subject.

A lipid-containing molecule can be incorporated into a liposome because the lipid portion will spontaneously integrate into the lipid bilayer. Thus, a lipid-containing agent may be presented on the “surface” of a liposome. Alternatively, an agent may be encapsulated within a liposome.

Formation of a liposome requires one or more lipids. Any lipids may be used which, singly or in combination, can form a liposome bilayer structure. Usually, these lipids will include at least one phospholipid. The phospholipids may be phospholipids from natural sources, modified natural phospholipids, semisynthetic phospholipids, fully synthetic phospholipids, or phospholipids (necessarily synthetic) with nonnatural head groups. The phospholipids of greatest interest are phosphatidyl cholines, phosphatidyl phosphatidyl ethanolamines, phosphatidyl serines, phosphatidyl glycerols, phosphatidic acids, and phosphatidyl inositols.

The liposome may include neutral, positively charged, and/or negatively charged lipids. Phosphatidyl choline is a neutral phospholipid. Phosphatidyl glycerol is a negatively charged glycolipid. N- [1-(2,3-dioleylox)propyl]-N,N,N-trimethylammonium chloride is a positively charged synthetic lipid. Another is 3-beta-[N-(N′,N″-dimethylaminoethane)-carbamoyl]-cholesterol.

Usually, the lipids will comprise one or more fatty acid groups. These may be saturated or unsaturated, and vary in carbon number, usually from 12-24 carbons. The phospholipids of particular interest are those with the following fatty acids: C12:0, C14:0, C16:0, C18:0, C18:1, C18:2, C18:3 (alpha and gamma), C20:0, C20:1, C20:3, C20:4, C20:5, C22:0, C22:5, C22:6, and C24:0, where the first number refers to the total number of carbons in the fatty acids chain, and the second to the number of double bonds. Fatty acids from mammalian or plant sources all have even numbers of carbon atoms, and their unsaturations are spaced at three carbon intervals, each with an intervening methylene group.

Cholesterol reduces the permeability of “fluid-crystalline state” bilayers.

A liposome may include lipids with a special affinity for particular target cells. For example, lactosylceramide has a specific affinity for hepatocytes (and perhaps also for liver cancer cells).

In a preferred, liposome formulation, the component lipids include phosphatidyl choline. More preferably they also include cholesterol, and still more preferably, also phosphatidyl glycerol. Taking advantage of the self-assembling properties of lipids, one or more immunogens may be attached to the polar lipids that in turn become part of the liposome particle. Each immunogen comprises one or more antigenic determinants (epitopes). These epitopes may be B-cell epitopes (recognized by antibodies) or T-cell epitopes (recognized by T-cells). The liposome can act to adjuvant the immune response elicited by the associated immunogens. It is likely to be more effective than an adjuvant that is simply mixed with an immunogen, as it will have a higher local effective concentration.

Moreover, a hapten may be attached in place of the aforementioned immunogen. Like an immunogen, a hapten comprises an antigenic determinant, but by definition is too small to elicit an immune response on its own (typically, haptens are smaller than 5,000 daltons). In this case, the lipid moiety may act, not only as an adjuvant, but also as an immunogenic carrier, the conjugate of the hapten and the lipid acting as a synthetic immunogen (that is, a substance against which humoral and/or cellular immune responses may be elicited).

Even if the lipid does not act as an immunogenic carrier, the liposome borne hapten may still act as a synthetic antigen (that is, a substance which is recognized by a component of the humoral or cellular immune system, such as an antibody or T-cell). The term “antigen” includes both haptens and immunogens.

Thus, in some embodiments, the invention contemplates a liposome whose membrane comprises a compound as disclosed herein, and at least one B-cell or T-cell epitope. The epitope may be furnished by a lipopeptide, glycolipid or glycolipopeptide.

The lipidation of an immunogen normally will facilitate the incorporation of the immunogen into a liposome, which in turn can improve the immune presentation of the immunogen. For most efficient incorporation, at least one strongly lipophilic group of the immunogen preferably should be similar in size to at least one of the lipid components of the liposome. For example, the size should be in the range of 50%-200% of the size of the reference lipid component of the liposome. Size may be measured by counting the number of non-hydrogen atoms of each, by calculating the molecular weight of each, or by calculating (with the aid of 3D molecular models) the molecular volume or longest dimension of each.

Preferably, the lipidated immunogen comprises a lipophilic moiety which adjuvants the humoral or cellular immune response to the immunogen.

Characterizing the Immune Response

The cell-mediated immune response may be assayed in vitro or in vivo. The conventional in vitro assay is a T cell proliferation assay. A blood sample is taken from an individual who suffers from the disease of interest, associated with that disease, or from a vaccinated individual. The T cells of this individual should therefore be primed to respond to a new exposure to that antigen by proliferating. Proliferation requires thymidine because of its role in DNA replication.

Generally speaking, T cell proliferation is much more extensive than B cell proliferation, and it may be possible to detect a strong T cell response in even an unseparated cell population. However, purification of T cells is desirable to make it, easier to detect a T cell response. Any method of purifying T cells which does not substantially adversely affect their antigen-specific proliferation may be employed. In our preferred procedure, whole lymphocyte populations would be first obtained via collection (from blood, the spleen, or lymph nodes) on isopycnic gradients at a specific density of 10.7, ie Ficoll-Hypague or Percoll gradient separations. This mixed population of cells could then be further purified to a T cell population through a number of means. The simplest separation is based on the binding of B cell and monocyte/macrophage populations to a nylon wool column. The T cell population passes through the nylon wool and a >90% pure T population can be obtained in a single passage. Other methods involve the use of specific antibodies to B cell and or monocyte antigens in the presence of complement proteins to lyse the non-T cell populations (negative selection). Still another method is a positive selection technique in which an anti-T cell antibody (CD3) is bound to a solid phase matrix (such as magnetic beads) thereby attaching, the T cells and allowing them to be separated (e.g., magnetically) from the non-T cell population. These may be recovered from the matrix by mechanical or chemical disruption.

Once a purified T cell population is obtained it is cultured in the presence of irradiated antigen presenting cells (splenic macrophages, B cells, dendritic cells all present). (These cells are irradiated to prevent them from responding and incorporating tritiated thymidine). The viable T cells (100,000-400,000 per well in 100 μl media supplemented with IL2 at 2.0 units) are then incubated with test peptides or other antigens for a period of 3 to 7 days with test antigens at concentrations from 1 to 100 μg/mL.

At the end of the antigen stimulation period a response may be measured, in several ways. First the cell free supernatants may be harvested and tested for the presence of specific cytokines. The presence of α-interferon, IL2 or IL12 are indicative of a Th helper type 1 population response. The presence of IL4, IL6 and IL10 are together indicative of a T helper type 2 immune response. Thus this method allows for the identification of the helper T cell subset.

A second method termed blastogenesis involves the adding tritiated thymidine to the culture (e.g., 1 μcurie per well) at the end of the antigen stimulation period, and allowing the cells to incorporate the radiolabelled metabolite for 4-16 hours prior to harvesting, on a filter for scintillation counting. The level of radioactive thymidine incorporated is a measure of the T -cell replication activities. Negative antigens or no antigen control wells are used to calculated the blastogenic response in terms of a stimulation index. This is CPM test/CPM control. Preferably the stimulation index achieved is at least 2, more preferably at least 3, still more preferably 5, most preferably at least 10.

CMI may also be assayed in vivo in a standard experimental animal, e.g., a mouse. The mouse is immunized with a priming antigen. After waiting for the T cells to respond, the mice are challenged by footpad injection of the test antigen. The DTH response (swelling of the test mice is compared with that of control mice injected with, e.g., saline solution.

Preferably, the response is at least 0.10 mm, more preferably at least 0.15 mm, still more preferably at least 0.20 mm, most preferably at least 0.30 mm.

The humoral immune response, in vivo, is measured by withdrawing blood from immunized mice and assaying the blood for the presence of antibodies which bind an antigen of interest. For example, test antigens may be immobilized and incubated with the samples, thereby capturing the cognate antibodies, and the captured antibodies then measured by incubating the solid phase with labeled anti-isotypic antibodies.

Preferably, the humoral immune response, if desired, is at least as strong as that represented by an antibody titer of at least 1/100, more preferably at least 1/1000, still more preferably at least 1/10,000.

Carrier

The compounds of the present invention can be formulated with a pharmaceutically acceptable carrier for injection or ingestion. The pharmaceutically acceptable carrier is a medium that does not interfere with the immunomodulatory activity of the active ingredient and is not toxic to the host to which it is administered. Pharmaceutically acceptable carriers include without limitation oil-in-water or water-in-oil emulsions, aqueous compositions, liposomes, micro beads and microsomes.

Pharmaceutical Subjects, Preparations and Methods

Applicants hereby incorporate by reference the discussion at pp. 32-46 of WO98/33810.

Subjects

The recipients of the vaccines of the present invention may be any vertebrate animal which can acquire specific immunity via a humoral or cellular immune response.

Among mammals, the preferred recipients are mammals of the Orders Primata (including humans, apes and monkeys), Arteriodactyla (including horses, goats, cows, sheep, pigs), Rodenta (including, mice, rats, rabbits, and hamsters), and Carnivora (including cats, and dogs). Among birds, the preferred recipients are turkeys, chickens and other members of the same order. The most preferred recipients are humans.

The preferred animal subject of the present invention is a primate mammal. By the term “mammal” is meant an individual belonging to the class. Mammalia, which, of course, includes humans. The invention is particularly useful in the treatment of human subjects, although it is intended for veterinary uses as well. By the term “non-human primate” is intended any member of the suborder Anthropoidea except for the family Hominidae. Such non-human primates include the superfamily Ceboidea, family Cebidae (the New World monkeys including the capuchins, howlers, spider monkeys and squirrel monkeys) and family Callithricidae (including the marmosets); the superfamily Cercopithecoidea, family Cercopithecidae (including the macaques, mandrills, baboons, proboscis monkeys, mona monkeys, and the sacred hunaman monkeys of India); and superfamily Hominoidae, family Pongidae (including gibbons, orangutans, gorillas, and chimpanzees). The rhesus monkey is one member of the macaques.

Pharmaceutical Compositions

Pharmaceutical preparations of the present invention, comprise at least one immunogen in an amount effective to elicit a protective immune response. The response may be humoral, cellular, or a combination thereof. The composition may comprise a plurality of immunogens.

At least one immunogen will be either a glycolipopeptide which is immunogenic per se, or a glycolipopeptide, which is immunogenic as a result of its incorporation into a liposome.

The composition preferably further comprises a liposome. Preferred liposomes include those, identified in Jiang,et al., PCT/US00/31281, filed Nov. 15, 2000 (our docket JIANG3A-PCT), and Longenecker, et al., Ser. No. 08/229,606, filed Apr. 12, 1994 (our docket LONGENECKER5-USA, and PCT/US95/04540, filed Apr. 12, 1995 (our docket LONGENECKER5-PCT).

The composition may comprise antigen-presenting cells, and in this case the immunogen may be pulsed onto the cells, prior to administration, for more effective presentation.

The composition may contain auxiliary agents or excipients which are known in the art. See, e.g., Berkow et al, eds., The Merck Manual, 15th edition, Merck and Co., Rahway, N.J., 1987; Goodman et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Katzung, ed. Basic and Clinical Pharmacology, Fifth Edition, Appleton and Lange, Norwalk, Conn. (1992), which references and references cited therein, are entirely incorporated herein by reference.

A composition may further comprise an adjuvant to nonspecifically enhance the immune response. Some adjuvants potentiate both humoral and cellular immune response, and other s are specific to one or the other. Some will potentiate one and inhibit the other. The choice of adjuvant is therefore dependent on the immune response desired.

A composition may include immunomodulators, such as cytokines which favor or inhibit either a cellular or a humoral immune response, or inhibitory antibodies against such cytokines.

A pharmaceutical composition according to the present invention may further comprise at least one cancer chemotherapeutic compound, such as one selected from the group consisting of an anti-metabolite, a bleomycin peptide antibiotic, a podophyllin alkaloid, a Vinca alkaloid, an alkylating agent, an antibiotic, cisplatin, or a nitrosourea. A pharmaceutical composition according to the present invention may further or additionally comprise at least one viral chemotherapeutic compound selected from gamma globulin, amantadine, guanidine, hydroxybenzimidazole, interferon-α, interferon-β, interferon-γ, thiosemicarbarzones, methisazone, rifampin, ribvirin, a pyrimidine analog, a purine analog, foscarnet, phosphonoacetic acid, acyclovir, dideoxynucleosides, or ganciclovir. See, e.g., Katzung, supra, and the references cited therein on pages 798-800 and 680-681, respectively, which references are herein entirely incorporated by reference.

Anti-parasitic agents include agents suitable for use against arthropods, helminths (including roundworms, pinlworms, threadworms, hookworms, tapeworms, whipworms, and Schistosomes), and protozoa (including amebae, and malarial, toxoplasmoid, and trichomonad organisms) Examples include thiabenazole, various pyrethrins, praziquantel, niclosamide, mebendazole, chloroquine HCl, metronidazole, iodoquinol, pyrimethamine, mefloquine HCl, and hydroxychloroquine HCl.

Pharmaceutical Purposes

A purpose of the invention is to protect subjects against a disease. The term “protection”, as in “protection from infection or disease”, as used herein, encompasses “prevention,” “suppression” or “treatment.” “Prevention” involves administration of a Pharmaceutical composition prior to the induction of the disease. “Suppression” involves administration of the composition prior to the clinical appearance of the disease. “Treatment” involves administration of the protective composition after the appearance of the disease. Treatment may be ameliorative or curative.

It will be understood that in human and veterinary medicine, it is not always possible to distinguish between “preventing” and “suppressing” since the ultimate inductive event or events may be unknown, latent, or the patient is not ascertained until well after the occurrence of the event or events. Therefore, it is common to use the term “prophylaxis” as distinct from “treatment” to encompass both “preventing” and “suppressing” as defined herein. The term “protection,” as used herein, is meant to include “prophylaxis.” See, e.g., Berker, supra, Goodman, supra, Avery, supra and Katzung, supra, which are entirely incorporated herein by reference, including all references cited therein.

The “protection” provided need not be absolute, i.e., the disease need not be totally prevented or eradicated, provided that there is a statistically significant improvement (p=0.05) relative to a control population. Protection may be limited to mitigating the severity or rapidity of onset of symptoms of the disease. An agent which provides protection to a lesser degree than do competitive agents may still be of value if the other agents are ineffective for a particular individual, if it can be used in combination with other agents to enhance the level of protection, or if it is safer than competitive agents.

The effectiveness of a treatment can be determined by comparing the duration, severity, etc. of the disease post-treatment with that in an untreated control group, preferably matched in terms of the disease stage.

The effectiveness of a prophylaxis will normally be ascertained by comparing the incidence of the disease in the treatment group with the incidence of the disease in a control group, where the treatment and control groups were considered to be of equal risk, or where a correction has been made for expected differences in risk.

In general, prophylaxis will be rendered to those considered to be at higher risk for the disease by virtue of family history, prior personal medical history, or elevated exposure to the causative agent.

Pharmaceutical Administration

At least one protective agent of the present invention may be administered by any means that achieve the intended purpose, using a pharmaceutical composition as previously described.

Administration may be oral or parenteral, and, if parenteral, either locally or systemically. For example, administration of such a composition may be by various parenteral routes such as subcutaneous, intravenous, intradermal, intramuscular, intraperitoneal, intranasal, transdermal, or buccal routes. Parenteral administration can be by bolus injection or by gradual perfusion over time. A preferred mode of using a pharmaceutical composition of the present invention is by subcutaneous, intramuscular or intravenous application. See, e.g., Berker, supra, Goodman, supra, Avery, supra and Katzung, supra, which are entirely incorporated herein by reference, including all references cited therein.

A typical regimen for preventing, suppressing, or treating a disease or condition which can be alleviated by an immune response by active specific immunotherapy, comprises administration of an effective amount of a pharmaceutical composition as described above, administered as a single treatment, or repeated as enhancing or booster dosages, over a period up to and including between one week and about 24 months.

It is understood that the effective dosage will be dependent upon the age, sex, health, and weight of the recipient, kind of concurrent treatment, if any, frequency of treatment, and the nature of the effect desired. The ranges of effective doses provided below are not intended to limit the invention and represent preferred dose ranges. However, the most preferred dosage will be tailored to the individual subject, as is understood and determinable by one of skill in the art, without undue experimentation. This will typically involve adjustment of a standard dose, e.g., reduction of the dose if the patient has a low body weight. See, e.g., Berkow et al, eds., The Merck Manual, 15th edition, Merck and Co., Rahway, N.J., 1987; Goodman et al., eds., Goodman and Gilman's The Pharmacological Basis of Therapeutics, 8th edition, Pergamon Press,: Inc., Elmsford, N.Y., (1990); Avery's Drug Treatment: Principles and Practice of Clinical Pharmacology and Therapeutics, 3rd edition, ADIS Press, LTD., Wiliams and Wilkins, Baltimore, Md. (1987), Ebadi, Pharmacology, Little, Brown and Co., Boston, (1985); Chabner et al., supra; De Vita et al., supra; Salmon, supra; Schroeder et al., supra; Sartorelli et al., supra; and Katsung, supra, which references and references cited therein, are entirely incorporated herein by reference.

Prior to use in humans, a drug will first be evaluated for safety and efficacy in laboratory animals. In human clinical studies, one would begin with a dose expected to be safe in humans, based on the preclinical data for the drug in question, and on customary doses for analogous drugs (if any). If this dose is effective, the dosage may be decreased, to determine the minimum effective dose, if desired. If this dose is ineffective, it will be cautiously increased, with the patients monitored for signs of side effects. See, e.g., Berkow, et al., eds., The Merck Manual, 15th edition, Merck and Co., Rahway, N.J., 1987; Goodman, et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985), which references and references cited therein, are entirely incorporated herein by reference.

The total dose required for each treatment may be administered in multiple doses (which may be the same or different) or in a single dose, according to an immunization schedule, which may be predetermined or ad hoc. The schedule is selected so as to be immunologically effective, i.e., so as to be sufficient to elicit an effective immune response to the antigen and thereby, possibly in conjunction with other agents, to provide protection. The doses adequate to accomplish this are defined as “therapeutically effective doses.” (Note that a schedule may be immunologically effective even though an individual dose, if administered by itself, would not be effective, and the meaning of “therapeutically effective dose” is best interpreted in the context of the immunization schedule.) Amounts effective for this use will depend on, e.g., the peptide composition, the manner of administration, the stage and severity of the disease being treated, the weight and general state of health of the patient, and the judgment of the prescribing physician.

Typically, the daily dose of an active ingredient of a pharmaceutical, for a 70 kg adult human, is in the range of 10 nanograms to 10 grams. For immunogens, a more typical daily dose for such a patient is in the range of 10 nanograms to 10 milligrams, more likely 1 microgram to 10 milligrams. However, the invention is not limited to these dosage ranges.

It must be kept in mind that the compositions of the present invention may generally be employed in serious disease states, that is, life-threatening or potentially life threatening situations. In such cases, in view of the minimization of extraneous substances and the relative nontoxic nature of the peptides, it is possible and may be felt desirable by the treating physician to administer substantial excesses of these peptide compositions.

The doses may be given at any intervals which are effective. If the interval is too short, immunoparalysis or other adverse effects can occur. If the interval is too long, immunity may suffer. The optimum interval may be longer if the, individual doses are larger. Typical intervals are 1 week, 2 weeks, 4 weeks (or one month), 6 weeks, 8 weeks (or two months) and one year. The appropriateness of administering additional doses, and of increasing or decreasing the interval, may be reevaluated on a continuing basis, in view of the patient's immunocompetence (e.g., the level of antibodies to relevant antigens).

A variety of methods are available for preparing liposomes, as described in, e.g., Szoka et al., Ann. Rev. Biophys. Bioeng. 9:467 (1980), U.S. Pat. Nos. 4,235,871, 4,501,728, 4,837,028, and 5,019,369, incorporated herein by reference.

The appropriate dosage form will depend on the disease, the immunogen, and the mode of administration; possibilities include tablets, capsules, lozenges, dental pastes, suppositories, inhalants, solutions, ointments and parenteral depots. See, e.g., Berker, supra, Goodman, supra, Avery, supra and Ebadi, supra, which are entirely incorporated herein by reference, including all references cited therein.

The antigen may be delivered in a manner which enhance, e.g., delivering the antigenic material into the intracellular compartment such that the “endogenous pathway” of antigen presentation occurs. For example, the antigen may be entrapped by a liposome (which fuses with the cell), or incorporated into the coat protein of a viral vector (which infects the cell).

Another approach, applicable when the antigen is a peptide, is to inject naked DNA encoding the antigen into the host, intramuscularly. The DNA is internalized and expressed.

It is also possible to prime autologous PBLs with the compositions of the present invention, confirm that the PBLs have manifested the desired response, and then administer the PBLs, or a subset thereof, to the subject.

Compound List

List of compounds (the alternate code given below is only to clarify those that have been used in the figures of biodata. No additional codes are introduced for those not mentioned in Figures or text.

| | alternate | | name |
|---|---|---|---|
| Compound | code | formula / MW | name |
| 1 | BC1-050 | C₃₅H₆₈NO₈ | (2R)-1-O-(α-D-galactopyranosyl)-2- |
| | 050 | 631.91 | hexacosanoylamino-propan-1,3-di-ol |
| 2 | BC1-038 | C₂₅H₄₉NO₈ | (2R)-1-O-(α-D-galactopyranosyl)-2- |
| | 038 | 491.65 | palmitoylamino-propan-1,3-di-ol |
| 3 | BC1-040 | C₃₇H₇₃NO₉ | (2R)-1-O-(α-D-galactopyranosyl)-2-(3- |
| | 040 | 675.96 | tetradecanoyloxytetradecanoyl)amino- |
| | | | propan-1,3-di-ol |
| 4 | BC-1548- | C₂₉H₄₉NO₈ | (2R)-1-O-(α-D-galactopyranosyl)-2- |
| | 03 | 539.69 | arachidonoylamino-propan-1,3-di-ol |
| 5 | BF-1508- | C₄₄H₇₇NO₈ | (2S,3R,4E)-1-(α-D-galactopyranosyloxy)-2- |
| | 84 | 748.06 | arachidonoylamino-3-hydroxy-4-octadecene |
| | BF 84 | | |
| 6 | BC1-041 | C₅₀H₉₇NO₈ | (2S,3R,4E)-1-(α-D-galactopyranosyloxy)-2- |
| | 041 | 840.28 | hexacosanoylamino-3-hydroxy-4-octadecene |
| 7 | BC1-049 | C₅₀H₉₉NO₈ | (2S,3R)-1-(α-D-Galactopyranosyloxy)-2- |
| | 049 | 842.30 | hexacosanoylamino-3-hydroxy-octadecane |
| 8 | BC1-046 | C₃₈H₅₆O₆ | 3-O-β-D-galactopyranosyl-cholesterol |
| | 046 | 548.78 | |
| 9 | BC1-051 | C₃₃H₅₆O₈ | 3-O-α-D-galactopyranosyl-cholesterol |
| | | 548.78 | |
| 10 | BC1-048 | C₃₅H₅₈O₆ | 3-O-β-D-galactopyranosyl-stigmasterol |
| | 048 | 574.81 | |
| 11 | BC1-047 | C₃₅H₅₈O₆ | 3-O-α-D-galactopyranosyl-stigmasterol |
| | 047 | 574.81 | |
| 12 | BC1-054 | C₃₅H₆₀O₆ | 3-O-β-D-galactopyranosyl-stigmasterol |
| | | 576.83 | |
| 13 | BC1-052 | C₃₅H₆₀O₆ | 3-O-α-D-galactopyranosyl-stigmasterol |
| | | 576.83 | |
| 033 | BC1-033 | C36H71NO7 | 1-O-(2-acetamido-2-deoxy-a-D- |
| | | 629.67 | galactopyranosyl)-3-tetradecanyloxy- |
| | | | tetradecan-1-ol |

### EXAMPLES

Preparation of Compound 15:

A mixture of N-Fmoc-serine allyl ester ( 96.0 g, 0.017 mol), stannous chloride (3.79 g, 0.02 mol), silver perchlorate (4.15 g, 0.20 mol), and molecular sieves 4 Å ( 2.0 g) in dry THF (30.0 mL) was stirred at room temperature for 20 minutes and cooled to −10° C. under nitrogen atmosphere. To the reaction mixture a solution of 2,3,3,4-O-tetra benzyl -D-galactopyranosyl fluoride 14 (11.05 g, 0.02 mol) in dry THF (25 mL) was added drop wise and stirred for 2 hrs at −10° C. The reaction mixture was filtered through celite, washed with ethyl acetate and solvent from combined filtrate distilled off. The residue was taken up in dichloromethane washed with saturated sodium bicarbonate, water and dried over anhydrous sodium sulphate. The solvent was distilled off and residue was chromatographed over silica gel and elution with hexane/ethyl acetate (4:1) gave 15 as white solid (6.01 g, 40% ) ¹H NMR (CDCl₃): δ 3.6 (m, 1H, Ser_{α}-H), 3.8-4.3 (m, 10H), 4.35-4.8 (m, 3H), 4.90-4.95 (d, 1H), 5.15-5.21(m,2H), 5.8-5.95 (m,1H, HC═), 6.35 (d, 1H, NH, 8.0 Hz), and 7.2-7.8 (m, 28H, Ar). ¹³C NMR: 99.77 (C-1).

Preparation of Compound 16:

To a solution of N-Fmoc serine (tetrabenzyl galactoparynosyl) allyl ester 15 (6.0 g, 0.0067 mol) in dry THF (60.0 mL) N-methyl aniline (1.46 mL, 0.0135 mol) was added under nitrogen. The reaction mixture was protected from light and tetrakis(triphenylphosphine) palladium (0) (0.780 g) was added. After stirring for 2 hrs. the solvent was distilled off and residue chromatographed on silica gel. Elution with dichloromethane/methanol/acetic acid (10:1:1) gave 16 as colorless solid (4.3 g, 75%). ¹H NMR (CDCl₃): δ 3.4-3.55 (m,2H), 3.7-3.8 (m, 2H), 3.9-4.0 (m, 3H), 4.1-4.2 (m, 3H), 4.35-4.6 (m, 5H), 4.7-4.98 (m, 5H), 6.25-6.30 (d,1H, NH, 7.0 Hz) and 7.3-7.8 (m, 28H, Ar)

Preparation of Compound 17:

To a solution of N-Fmoc (tetrabenzyl-α-D-galactopyranosyl) serine 16 (4.3 g, 0.0051 mol) in dry dichloromethane (40.0 mL) dry pyridine was added and cooled to −15° C. under nitrogen. Cyanuric fluoride (0.92 mL, 0.01 mol) was added and reaction mixture stirred at −15° C. for one hour followed by addition of dichloromethane and reaction mixture allowed to warm to room temperature. It was washed with cold water (100 mL), dried over anhydrous sodium sulphate and solvent distilled off. The residue was dissolved in dry dichloromethane (50 mL) and, with stirring under nitrogen, a solution of 2M sodium borohydride in triethylene glycol dimethyl ether (5.1 mL) was added. After stirring for 1.5 hrs. at room temperature the reaction mixture was quenched with 0.5 M sulfuric acid (5.0 mL) and diluted with methylene chloride. The organic phase was washed with 0.5M sulfuric acid, saturated sodium bicarbonate, water and dried. After distilling of the solvent the residue was chromatographed on silica gel and elution with hexane/ethyl acetate (3:2) gave 17 as colorless solid (2.84 g, 67%). ¹H NMR (CDCl₃): δ 3.2-3.3 (m, 1H), 3.35-3.4 (m, 3H), 3.85-3.55 (m, 5H), 4.0-4.05 (m, 1H), 4.2 (t, 1H), 4.3-4.45 (m, 4H), 4.5-4.9 (m,7H, 3×CH₂Ph & H-1), 5.68 (d, 1H, NH, J=7.5 Hz), 7.2-7.8 (m, 28H, Ar).

Preparation of Compound 18:

The N-Fmoc amino serinol derivative 17 (780 mg, 0.933 mmol) was dissolved in morpholine. (20 mL) and stirred at room temperature for 2 hrs. The solvent was distilled off using toluene as co-solvent and the residue was chromatographed. Elution with/hexane/ethyl acetate/methanol (10:10:4) gave free amine 18 as yellow syrup (634 mg). ¹H NMR (CDCl₃+CD₃OD): δ 3.35-3.50 (m, 4H), 3.55-3.8 (m), 4.0-4.05 (m,1H), 4.4-4.9 (md, 9H, 4×CH₂Ph, & H-1), and 7.3-7.4 (m, 40H, Ar).

Prepartion of Compound 22:

The N-Fmoc amino serinol derivative 17 (320 mg, 0.38 mmol) was dissolved in a solution of 0.1M TEAF in THF (20.0 mL) and stirred at room temperature to form in situ the free amine 18. In a separate round bottom flask a mixture of hexacosanoic acid 19 (285 mg, 0.72 mmol), TBTU (231 mg, 0.72 mmol), HOBt (97 mg, 0.72 mmol) and triethyl amine (167 μL, 1.20 mmol) was stirred in dry DMF and heated at 40° C. under nitrogen for 15 minutes. To this reaction mixture was added the solution of free amine 18 drop wise and the reaction mixture was heated at 40° C. overnight under nitrogen. The reaction mixture was diluted with dichloromethane (100 mL) and ice-cold water (300 mL) and extracted with dichloromethane three times (100 mL). The combined organic extract was washed with cold water and dried over anhydrous sodium sulphate. The solvent was distilled off and residue purified on silica gel. Elution with hexane/ethyl acetate/methanol (10:10:0.2) gave 22 as light yellow solid (165 mg, 44%). ¹HNMR (CDCl₃): δ 0.9 (t, 3H, CH₃), 1.3 (br s, 43H), 1.6 (rm, 2H), 1.75-1.80 (br s, 1H), 1.85-1.88 (m, 4H), 2.1 (m, 2H), 3.4-3.52 (m, 2H), 3.55-3.62 (m, 2H), 3.65 (brs, 1H), 3.72-3.8 (m, 2H), 3.82-3.9 (m,5H), 3.95 (m, 1H), 4.0-4.05 (m, 3H), 4.35-4.5 (q, 2H, CH₂Ph, J=12 Hz), 4.55-4.68 (q, 2H, CH₂Ph, J=12 Hz), 4.72 (d,1H, H-1, J=3.5 Hz), 4.75-4.95 (m, 4H, 2×CH₂Ph), 6.35 (d,1H, NH, J=8.0 Hz) and 7.25-7.4 (m, 20H, Ar).

Prepartion of Compounds 23:

A mixture of 2-amino serinol derivative 18 (207 mg, 0.375 mmol), sodium bicarbonate (38 mg, 0.450 mmol), palmitoyl succinimide 20 (168 mg, 0.450 mmol) in THF and water (1:1, 10 mL) was stirred overnight at room temperature. The solvent was distilled off and residue dissolved in dichloromethane, washed with water and organic phase dried over anhydrous sodium sulphate. The solvent was distilled off and residue chromatographed on silica gel. Elution with hexane/ethyl acetate/methanol (10:10:0.03) gave 23 as colorless solid (240 mg, 62%). ¹HNMR (CDCl₃): δ 0.9 (t, 3H, CH₃), 1.2-1.3 (brs, 25H, alkyl CH), 1.5-1.65 (m, 3H), 2.1 (t, 1H), 3.4-3.7 (m, 7H), 3.8-4.0 (m, 5H), 4.4-4.68 (4d, 4H, 2×CH₂Ph, J=12.0 Hz), 4.73 (d,1H, H-1, J=3.5 Hz), 4.75-4.9 (4d, 4H, 2×CH₂Ph, J=12.0 Hz), 6.4 (d, 1H, NH, J=8.0 Hz), 7.3-7.45 (m, 20H, Ar).

Prepartion of Compound 24:

A mixture of 3-tetradecyloxy myristic acid 21 (116 mg, 0.263 mmol, 4-methylmorpholine (30 μL) in dry THF (2 mL) was cooled to −20° C. with stirring under nitrogen for 10 minutes and then isobutylchloroformate (37 μL, 0.289 mmol) was added and the reaction mixture stirred for another 15 minutes. To this mixture a solution of amino serinol derivative 18 (194 mg, 0.263 mmol) in dry THF (2 mL) and 4-methylmorpholine (30 μL) was added drop wise and reaction mixture stirred for 1 hr at −20° C. The reaction was quenched with methanol (2 mL) reaction mixture allowed to warm up to room temperature and solvent distilled off. The residue was chromatographed on silica gel and elution with hexane/ethyl acetate/methanol (20:10:0.5) gave 24 as white solid (197 mg, 72%). ¹H NMR (CDCl₃): δ 0.9 (t, 6H, 2×CH₃), 1.2 (br s, 33H, alkyl CH₂), 1.4-1.6 (m, 5H, OH and 2×CH₂), 2.35 (m, 2H), 3.4-3.7 (m, 7H), 3.85-3.95 (m, 5H), 4.05-4.15 (m, 2H), 4.4-4.95 (md, 9H), and 7.35-7.45 (m, 20H, Ar).

Preparation of Compound 1:

The glycolipid 22 (160 mg, 0.161 mmol) was dissolved in mixture of ethyl acetate/methanol/acetic acid (75 mL/5 mL/7 mL) and hydrogenated in the presence of Pd—C (10%) and followed by TLC. After 72 hrs reaction mixture showed absence of the starting compound, and catalyst was filtered through celite and washed with chloroform/methanol (5:1). The solvent from combined filtrate was distilled off, residue chromatographed on silica gel and elution with chloroform/methanol (4:1) gave 1 as colorless solid (50 mg, 50%). ¹H NMR (CDCl₃+CD₃OD): δ 0.9 (t, 3H, CH₃), 1.25 (br s, 41H, allyl CH), 1.60-1.65 (m, 2H), 2.2-2.25 (t, 2H, CH₂), 3.59-3.69 (m,3H), 3.72-3.82 (m, 6H), 3.95 (d,1H, H-4, J=1.25 Hz), 4.04-4.08 (m,1H) and 4.9 (d, 1H, H-1, J=2.5H_{z}). C₃₅H₆₉NO₈ (631.5). ESIMS found 654.5 (M+Na).

Preparation of Compound 2:

A mixture of 23 (214 mg, 0.251 mol) and Pd—C (10%, 125 mg) in ethyl acetate/methanol/acetic acid (75 mL/5 mL/7 mL) was hydrogenated with stirring for 24 hrs. The catalyst was filtered through celite and washed with chloroform/methanol/water (80:20:3). The solvent from combined filtrate was distilled off using toluene as co-solvent, the residue was chromatographed and elution with chloroform/methanol (3:1) gave 2 as white solid (100 mg, 81%). ¹H NMR (CD₃OD): δ 0.9 (t, 3H, CH₃), 1.3-1.4 (br s, 25H, alkyl CH₂), 1.6 (br t, 2H), 2.15-2.25 (t, 2H), 3.57-3.62 (m, 2H), 3.65-3.69 (m, 2H), 3.74-3.79 (m,4H), 3.85 (dd, 1H), 4.5 (m,1H), 4.84 (d,1H, H-1, J=3.5 Hz).

Preparation of Compound 3:

The tetrabenzyl-α-D-Glactopyranoside serinol derivative 9 (180 mg, 0.174 mmol) was hydrogenated in the presence of Pd—C (10%, 125 mg) in mixture of ethyl acetate/methanol/acetic acid (75 mL, 5 mL, 7 mL) for 24 hrs. The catalyst was filtered through celite and washed with chloroform/methanol/water (100 mL, 80:20:3). The solvent from combined filtrate was distilled off and residue purified on silica gel. Elution with chloroform/methanol (4:1) gave 3 as white solid (83 mg, 71%). ¹H NMR (CD₃OD): δ 0.89 (t, 6H, 2×CH₃), 1.4-1.48 (br s, 37H, alkyl CH₂), 1.49-1.6 (brt, 4H), 2.3 (dd, 1H, J=5.5 Hz & 12.0 Hz), 2.4-2.5 (m, 1H), 3.4-3.55 (m, 2H), 3.6-3.8 (m, 10H), 3.9 (m,1H), 4.10 (t,1H) and 4.85 (d, 1H, H-1, J=3.5 Hz). ¹³C NMR: 14.45 (CH₃), 101.06 (C-1), and 174.08 (C═O).

Prepartion of Compound 26:

Compound 25 (11.10 g, 50.45 mmol) was treated with benzaldehyde dimethyl acetal (15.1 mL, 100.9 mmol) and p-toluenesulfonic acid (479 mg, 2.52 mmol) in dry acetonitrile (100 mL) at room temperature overnight. Triethylamine (1.0 mL) was added and the mixture was concentrated in vacuo. The residue was purified by flash chromatography (dichloromethane:methanol, 100:2.5 and 100:5) to give 26 (9.5 g, 60%).

Preparation of Compound 27:

To a solution of allyl-4,6-O-benzylidene-β-D-galactopyranoside 26 (22.66 g, 0.073 mol) in dry DMF, under nitrogen atmosphere and with stirring at 0° C., sodium hydride (95%; 4.4 g, 0.183 mol) was added in small portions over a period of 30 minutes and stirred for another 45 minutes. A solution of p-methoxy benzyl chloride (24.83 mL, 0.183 mol) was added drop wise, reaction mixture allowed to warm to room temperature and stirred over night. The reaction was quenched by adding methanol (25 mL) drop wise and solvent distilled off under high vacuum. The residue as syrup was dissolved in dichloromethane (250 mL), washed with water (3×250 mL) and organic extract dried over anhydrous sodium sulphate. The solvent distilled off to a get solid which was crystallized from ether/hexane to get 27 as colorless solid (25.89 g, 64%). ¹H NMR (CDCl₃+CD₃OD): δ: 3.45 (dd, 1H,J=3.5 & 10.5 Hz), 3.7-3.85 (m, 10H, 2×OCH₃ & other protons), 4.15-4.2(m, 1H), 4.3 (dd, 1H, J=2.0 & 12.0 Hz), 4.4-4.5 (m, 2H), 4.68-4.71 (m, 1H), 4.85 (br d, 1H, J=10.0 Hz, H-1), 5.2-5.25 (m, 1H), 5.32-5.4 (br m, 1H, CH═CH,), 5.5 (s, 1H, CHPh), 5.92-6.04 (m, 1H, CH═CH), 6.8-6.9 (m, 4H, Ar), 7.3-7.4 (m,7H, Ar) and 7.55-7.6 (m, 2H, Ar).

Preparation of Compound 28:

Hydrogen gas was bubbled in to a solution of Ir(I) catalyst (231 mg 0.27 mmol) in dry THF (75 mL) till a clear yellow solution was obtained and this was transferred to a solution of allyl glycoside 27 (15.0 g, 0.027 mol) in dry THF (75 mL) and the mixture was stirred under nitrogen atmosphere at room temperature for 2 hrs. To the reaction mixture N-bromosuccinimide (7.2 g, 0.041 mol) added and stirred in dark at room temperature for two hours. The solvent was distilled and the syrup dissolved in dichloromethane (200 mL), washed with water (3×200 mL) and organic extract dried over anhydrous sodium sulphate. The solvent was removed in vacuo, residue chromatographed on silica gel, using dichloromethane/ethyl acetate (10:1), to get 28 as colorless solid (9.63 g, 69%). ¹H NMR (CDCl₃): δ 2.92 (d, 1H, J=2.5 Hz) 3.1 (d, 1H, J=7.0H_{z}), 3.55 (dd, 2H, J=3.5 H_{z} & 11.0 H_{z}), 3.8-3.95 (m, 8H), 3.99-4.05 (m, 2H), 4.15-4.25 (m, 2H), 4.6-4.8 (m,5H), 5.35(m, 1H), 5.5 (s, 1H, CHPh), 6.85 (m, 4H, Ar), 7.25-7.35 (m, 7H, Ar) and 7.55 (m, 2H, Ar).

Preparation of Compound 29:

To a mixture of compound 28 (9.63 g, 0.019 mol) in dry dichloromethane (250 mL) and trichloroacetonitrile (19 mL), with stirring and under nitrogen, DBU (1.40 mL) was added drop wise at room temperature. The reaction was analyzed by TLC and after 2 hrs and the solvent distilled off and residue chromatographed with hexane/ethyl acetate (3:1) to get 29 as colorless solid (5.0 g, 40%). ¹H NMR(CDCl₃): δ 3.8 (s, 6H, 2×OCH₃), 4.0-4.1 (m,2H), 4.2-4.3 (m,3H), 4.68-4.75 (m, 4H), 5.5 (s, 1H, CHPh), 6.6 (d,1H, H-1, J=3.5 Hz), 6.8-7.6 (m,13H, Ar), and 8.6 (s, 1H, NH).

Preparation of Compound 31:

A mixture of trichloroacetimidate 29 (100 mg, 0.153 mmol), N-Fmoc-serine phenacyl ester 30 (81.0 mg, 0.184 mmol) and molecular sieves 4 Å (0.5 g) in dry THF (2 mL) was stirred for 10 minutes at room temperature under nitrogen atmosphere and then cooled to 0° C. To the reaction mixture a solution of TMSOTf (0.01 M, 0.0153 mmol) in dry THF was added drop wise very slowly and stirred for 30 minutes at 0° C. The reaction mixture was quenched with triethyl amine, filtered through celite and washed with THF. The solvent from the combined filtrate was distilled off and residue chromatographed. Elution with toluene/acetone (10:1) gave 31 as white solid (77 mg, 54%). ¹H NMR (CDCl₃) δ: 3.7-3.8 (m, 7H, 2×OCH₃ & Ser-α), 3.9 (m, 4H), 4.2-4.3 (m, 4H), 4.35-4.45 (m, 2H), 4.6 (d, 1H, J=12.0 Hz), 4.66-4.79 (m, 4H), 497 (d, 1H, J=3.0 Hz, H-1), 5.32 (br s, 2H), 5.48 (s, 1H, CHPh), 6.2 (d, 1H, J=8.0 Hz, NH), 6.8-6.85 (m, 4H, Ar), 7.3-7.42 (m, 11H, Ar), 7.45-7.55 (m, 4H, Ar), 7.57-7.61 (m, 3H, Ar) and 7.74-7.84 (m, 4H, Ar).

Preparation of Compound 32:

To a solution of 31 (14.94 g) in 80% acetic acid/toluene (750 mL) activated zinc dust (20.81 g) was added and reaction mixture stirred at room temperature and reaction was followed by TLC (hexane/ethyl acetate/methanol/acetic acid, 10:10:1:1). Zinc dust was filtered off on celite and washed several times with methylene chloride. The solvent from the combined filtrate was distilled off and the yellowish off white residue was chromatographed. Elution with methylene chloride methanol/acetic acid (40:1:0.1) gave 32 as white solid (2.72 g, 25%). ¹H NMR (CDCl₃+CD₃OD) δ: 3.75-3.95 (m, 10H, 2×OCH₃, & other protons), 4.05-4.25 (m, 5H), 4.35-4.40 (m,3H), 4.60-4.70 (m, 4H), 4.85 (d, 1H, J=10.0 Hz), 4.90 (d, 1H, J=3 Hz,H-1). 5.5 (s, 1H, CHPh), 6.82-6.80 (m, 4H, Ar—OCH₃), 7.25-7.42 (m, 11H, Ar), 7.48-7.6 (m, 4H, Ar) and 7.22-7.77 (m, 2H, Ar).

Preparation of Compound 33:

A solution of serine compound 32 (800 mg, 0.734 mmol) in mixture of dry methylene chloride (15 mL) and pyridine (90 μL) was cooled to −15° C. under nitrogen atmosphere and cyanuric fluoride (99 μL, 1.10 mmol) was added and reaction mixture was stirred for 2 hours. It was diluted with methylene chloride and organic extract washed with cold water and dried over anhydrous sodium sulphate. The solvent was distilled off and the residue, as white foam, dissolved in methylene chloride (5 mL) and to it 2 M sodium borohydride solution in triethylene glycol dimethyl ether (0.245 mg, 0.490 mmol) was added at room temperature and stirred for 2 hours. The reaction was quenched by adding water, diluted with methylene chloride, washed with water, saturated sodium bicarbonate and with water again. The organic extract was dried over anhydrous sodium sulphate and solvent distilled off. The residue was chromatographed and elution with hexane/ethyl acetate/methanol, (10:10:0.5) gave 33 as white solid (770 mg, 98%).

Preparation of Compound 34:

A mixture of 33 (640 mg) in methylene chloride (32 mL) and 95% aq. TFA (1.6 mL) was stirred at room temperature and reaction followed by TLC hexane/ethyl acetate/methanol (10:10:1). After one hour the reaction was quenched with saturated sodium bicarbonate, diluted with water (100 mL) and extracted with methylene chloride (3×50). The aqueous extract was freeze dried, residue chromatographed on LH-20 and elution with ethanol gave 34 as white solid (270 mg, 67%). ¹H NMR (CDCl₃+CD₃OD) δ: 3.4-3.64 (m,10H), 3.71 (d, 1H 2.5 Hz), 4.0 (brt,1H), 4.17 (br d, 2H, J=7.5 Hz), 4.75 (d, 1H; J=3.0 Hz, H-1), 7.1-7.25 (m, 4H, Ar), 7.38-7.40 (m, 2H, Ar) and 7.55-7.6 (m, 2H, Ar).

Preparation of Compound 4:

A mixture of N-Fmoc serinol compound 34 (50 mg) and morpholine(1 mL) was stirred at room temperature and after one hour the solvent was distilled off using toluene as co-solvent and the residue, as yellow solid, was dried under high vacuum for 2 hours. The solid was dissolved in a mixture of acetone/water (1:1, 2 ml) and stirred with sodium bicarbonate (11 mg, 0.126 mmol) and arachidonyl succinimide ester 36 (51 mg, 0.126 mmol) and stirred, under dark condition, at room temperature for overnight. The solvent was distilled off under high vacuum and the residue chromatographed on silica gel. Elution with chloroform/methanol/water (8:1:0.1) gave 4 as viscous syrup (44 mg, 75%). ¹H NMR (CDCl₃+CD₃OD) δ: 0.9 (t, 3H, J=7.0 Hz, CH₃), 1.2-1.35 (m, 8H, CH₂), 1.68-1.72 (m, 2H), 2.03-2.15 (m, 4H), 2.21-2.25 (m, 2H), 2.6 (s, 1H), 2.79-2.85 (m, 5H), 3.57-3.61 (m, 2H), 3.67-3.82 (m, 8H), 3.95 (d, 1H, J=3.5 Hz, H-1), 4.03-4.07 (m, 1H), 4.9 (s, 1H) and 5.33-5.4 (m, 7H, CH═CH).

Preparation of Compound 36:

Arachidonic acid 35 (300 mg, 0.985 mmol), N-hydroxysuccinimide (NHS, 125 mg, 1.08 mmol) and DCC (223 mg, 1.08 mmol) were dissolved in ethyl acetate (10 mL) and the mixture was stirred at room temperature for 16 h. The solid was filtered and the solid washed with ethyl acetate. The filtrate was concentrated in vacuo and the residue purified by flash chromatography (hexane:ethyl acetate, 4:1) to give 36 (243 mg; 61%).

Preparation of Compound 38:

A mixture of D-erythro-sphingosine 37 (2.26, 7.55 mmol), and sodium bicarbonate (761 mg, 9.06 mmol), in a mixture of acetone/water (40 mL, 1:1) was stirred at room temperature for 30 minutes and the Fmoc-N-hydroxy succinimide (3.04 g, 9.06 mmol) added and stirring continued for 70 hrs. The acetone was distilled off, water (200 mL) added and extracted with dichloromethane. The organic extracted was dried over anhydrous, solvent distilled off and residue was chromatographed, dichloromethane/ethyl acetate (2:1) to get 38 as colorless solid (2.8 g, 71%). ¹H NMR (CDCl₃): δ 0.89 (t, 3H J=7.5 Hz CH₃), 1.25 (br s, 22H, alkyl CH₂), 1.65 (br s, 1H, OH), 2.05 (m, 2H, ═CH—CH₂), 2.45 (br s, 1H, OH), 3.6-3.75 (m, 2H), 3.95-4.12 (m, 1H), 4.2-4.25 (t, 1H, J=7.5 H_{z}), 4.34-4.45 (m, 3H), 5.5-5.6 (m, 2H, NH, HC═), 5.8-5.9 (m, 1H, HC═), 7.3-7.45 (m, 4H, Ar), 7.6 (d, 2H, Ar), 7.75 (d, 2H, Ar).

Preparation of Compound 39:

Trityl chloride (5.57 g, 20.0 mmol) was added to a mixture of N-Fmoc-sphingosine 38 (2.61 g, 5.00 mmol) in dry pyridine (30 mL) and DMAP (183 mg, 1.5 mmol) at room temperature and stirred for 48 hrs. The solvent was distilled under reduced pressure, to the residue water (300 mL) added and extracted with dichloromethane. The organic phase was washed with water three times (100 mL), dried over anhydrous sodium sulphate, solvent distilled off using toluene as co solvent to remove trace amount of pyridine. The yellow solid was chromatographed with hexane/ethyl acetate (10:1) to get 39 as light yellow solid (2.89 g, 76%). ¹H NMR (CDCl₃): δ 0.88 (t, 3H, J=7.0 H_{z}, CH₃), 1.25 (br s, 22H, alkyl CH₂), 1.95 (m, 2H, ═CHCH₂), 2.95 (d, 1H, J=7.0 H_{z}), 3.29-3.34 (dd, 1H), 3.39-3.44 (dd, 1H), 3.80 (br s, 1H), 4.23-4.30 (m, 2H), 4.35-4.43 (m, 2H), 5.25-5.30 (dd, 1H, H-4), 5.45 (d, 1H, J=7.5 H_{z}, NH), 5.64-5.70 (m, HC═) and 7.2-7.6 (m, 23H, Ar).

Preparation of Compound 40:

To a stirred mixture of N-Fmoc-amino-1-O-trityl-D-erythro-sphingosine 39 (2.89 g, 3.78 mmol) in dry pyridine (30 mL) and DMAP (92 mg, 0.757 mmol), at room temperature benzoyl chloride (91.32 mL, 11.34 mmol) was added drop wise and allowed to stir for 18 hrs The solvent was distilled off and residue extracted in dichloromethane, washed with water three times (100 mL) and dried over anhydrous sodium sulphate. The solvent was removed and traces of pyridine distilled of using toluene as co solvent to get a syrup which was chromatographed with hexane/ethyl acetate (20:1) gave 40 (3.01 g, 92%). ¹H NMR (CDCl₃): δ 0.85 (t, 3H, J=7.0 Hz, CH₃), 1.2-1.3 (br s, 22H, alkyl CH₂), 2.05 (m, 2H, ═HCCH₂), 3.25 (m, 1H), 3.45 (m, 1H), 4.15-4.4 (m, 4H), 5.15 (d, 1H,J=9.0 H_{z}), 5.45 (dd, 1H, J=12.5 Hz & 7.0 Hz) 5.75 (m, 1H), 5.90 (m, 1H, CH═CH), 7.2-7.5 (m, 24H, Ar), and 7.8-7.80 (m, 4H, Ar).

Prepartion of Compound 41:

To a solution of protected sphingosine derivative 40 (2.89 g, 3.83 mmol) in a mixture of dry dichloromethane/methanol (30 mL, 2:1), with stirring at room temperature, p-toluene sulfonic acid (317 mg, 1.66 mmol) was added and reaction was followed by TLC. After four hrs. the reaction was quenched by triethyl ethyl amine. The solvent was distilled off and residue chromatographed with dichloromethane/ethyl acetate (20:1) to get 41 as white solid (1.38 g, 66% ). ¹H NMR (CDCl₃): δ 0.85 (t, 3H, J=7.5 H_{z}, CH₃), 1.2-1.4 (br s, 21H, alkyl CH₂), 2.0-2.05 (m, 2H, ═CHCH₂), 2.55 (m, 1H), 3.75 (m, 1H), 4.25(t, 1H, J=7.0 Hz), 4.31-4.4 (m, 2H), 5.3-5.4 (br d, 1H, J=9.0 Hz), 5.5-5.7 (m, 2H), 5.85-5.95 (m, 1H, HC═), 7.3-7.45 (m, 6H, Ar), 7.5 (m, 3H, Ar), 7.85 (m, 2H, Ar) and 8.10 (m, 2H, Ar).

Preparation of Compound 42:

A mixture of the trichloroacetimidate 29 (1.83 g, 2.80 mmol), 2-N-Fmoc-sphingosine 41 (1.17 g, 1.87 mmol) and molecular sieves 4 Å (500 mg) in dry THF (20 mL) was stirred at room temperature under nitrogen for 1 h and cooled to −10° C. To the reaction mixture a solution of TMSOTf (0.01 M, 56 μL in 28 mL of THF) was added drop wise and stirred at −10° C. The reaction was followed by TLC, quenched with triethyl amine after 30 minutes, filtered on celite and washed with methylene chloride. The solvent from the combined filtrate was distilled off and residue was chromatographed. Elution with toluene:acetone (30:1) gave 42 (1.33 g, 59%). ¹H NMR (CDCl₃) δ: 0.89 (t, 3H, J=7.5 Hz, CH₃), 1.25 (br d, 22H, CH₂), 1.95 (m, 2H, CH₂), 3.6 (br s, 1H), 3.7-3.8 (m, 8H, 2×OCH₃ & 2H), 3.90-4.05 (m, 3H), 4.10-4.3.(m, 5H), 4.44-4.45 (m, 1H), 4.6-4.75 (m, 4H), 4.89 (d, 1H, J=3.0 Hz, H-1), 5.35 (d, 1H, J=8.5 Hz, NH), 5.45 (s, 1H, CHPh), 5.59-5.65 (m, 2H), 5.80-5.90 (m, 1H, HC═), 6.9 (m, 4H, Ar), 7.3-7.6 (m, 18H), 7.75-7.85 (m, 2H, Ar), and 8.05-8.81 (m, 2H, Ar).

Preparation of Compound 43:

A mixture of arachidonic acid 35 (162 μL, 0.491 mmol), TBTU (158 mg, 0.491 mmol), HOBT (66.0 mg, 0.491 mmol) and N-methylmorpholine (98 μL, 0.892 mmol) in dry THF (10 mL) was stirred, in a three necked flask fitted with a dropping funnel, at room temperature under nitrogen atmosphere for 15 minutes. In a separate flask α-Gal-N-Fmoc-sphingosine 42 (498 mg, 0.446 mmol) was stirred with 0.1M tetra butyl ammonium fluoride solution in THF (10 mL) for 5 minutes and then transferred to the dropping funnel, solution added to the reaction mixture drop wise and stirred overnight. The reaction was followed by TLC (toluene:acetone, 10:1), solvents distilled off under high vacuum and the residue was chromatographed. Elution with hexane and ethyl acetate (3:1 and 0.1% acetic acid) gave 43 as light yellow solid (391 mg, 74.0%). ¹H NMR (CDCl₃) δ: 0.94 (t, 6H,J=7.5 Hz,2×CH₃), 1.212-1.38 (m, 32H, CH₂), 1.62-1.65 (m, 5H), 1.98-2.1 (m, 8H), 2.78-2.84 (m, 6H), 3.75-3.8 (m, 8H, 2×OCH₃ and others H), 3.9-3.41 (m, 4H), 4.18-4.2 (m, 2H), 4.5-4.55 (m, 1H), 4.82-4.90 (m, 8H), 4.95 (d, 1H, J=3.0 Hz,H-1), 5.32-5.42 (m, 8H, HC═CH), 5.46-5.5 (m, 2H, CHPh and other proton ),5.58-5.60 (m, 1H, HC═), 5.74-5.8 (m,1H, HC═), 6.05 (d,1H, J=8.0 Hz, HN), 6.78-6.88 (m, 4H, Ar), 7.3-7.38 (m, 12H, Ar) and 8.02-8.06 (m, 2H, Ar).

Preparation of Compound 44:

A mixture of hexacosanoic acid 19 (235 mg, 0.591 mmol), TBTU (190 mg, 0.591 mmol, HOBT (80.0 mg, 0.591 mmol) and n-methyl morpholine (130 μL, 1.182 mmol) in dry DMF (10 mL) was stirred, in a three necked flask fitted with a dropping funnel, at 40° C. under nitrogen atmosphere for 15 minutes. In a separate flask α-Gal-N-Fmoc-sphingosine 42 (600 mg, 0.537 mmol) was stirred with 0.1 M tetra butyl ammonium fluoride solution in THF (12 mL) for 2 minutes and then transferred to the dropping funnel, solution added to the reaction mixture drop wise and stirred at 40° C. The reaction was followed by TLC (hexane:ethyl acetate, 2:1) and after 16 hrs solvents distilled off under high vacuum and the residue was chromatographed. Elution with toluene:acetone (20:1) gave 44 as white solid (463 mg, 68%). ¹H NMR (CDCl₃+CD₃OD) δ: 0.86-0.88 (m, 6H, 2×CH₃), 1.2-1.3 (br s, 58H, CH₂), 1.51-1.54 (m, 4H), 1.95-2.1 (m, 7H), 3.75-3.8 (2s, 6H, 2×OCH₃), 3.87-3.91 (m, 1H), 3.95-3.98 (dd, 1H, J=2.0 Hz & 12.0 Hz), 4.0-4.04 (d, 1H, J=3.5 & 10.5 Hz), 4.43-4.47 (m, 1H), 4.62-4.65 (d, 2H, J=12.0 Hz,CH₂), 4.69-4.72 (d, 2H, J=12 Hz,CH₂), 4.88 (d, 1H, J=3.5 Hz, H-1), 5.43-5.48 (m, 2H), 5.57 (t, 1H, J=7.0 Hz), 5.73-5.78 (m, 1H, CH═CH), 5.99. (d,1H, J=8.5 Hz, NH), 6.78-6.85 (m, 4H, Ar), 7.28-7.35 (m, 7H, Ar), 7.42-7.50 (m, 4H, Ar), 7.54-7.58 (m, 1H, Ar) and 8.0-8.05 (m, 2H, Ar).

Preparation of Compound 45:

The compound 43 (325 mg, 0.275 mmol) was dissolved in dry THF (5 mL) and treated with 1M solution of sodium methoxide (5 mL) at room temperature and followed by TLC (toluene:methanol, 10:1). The reaction mixture was treated with weak acid resin to pH5-6, filtered and washed the resin with THF several times. The solvent from the combined filtrate was distilled off and residue chromatographed. Elution with toluene and methanol (10:1) gave 45 (270 mg, 91%). ¹H NMR (300 MHz, CDCl₃+CD₃OD) δ: 0.95 (t, 6H, J=7.0 Hz, 2×CH₃), 1.25-1.36 (m, 30H, CH₂), 1.56-1.58 (br s, 3H), 1.65-1.71 (m, 2H), 1-98-2.10 (m, 6H), 2.15-2.18 (br t, 2H), 2.80-2.85 (m, 6H), 3.67-3.70 (m, 2H), 3.79-3.80 (2s, 6H, 2×OCH₃), 3.88-3.91, (m, 2H), 3.95-4.05 (m, 3H), 4.11-4.15 (m, 1H), 4.16-4.19 (dd, 1H, 4.63(d 1H), 4.69 (br s, 2H), 4.81-4.83(m, 2H), 5.32-5.44 (m, 8H, HC═), 5.46 (s, 1H, CHPh), 5.63,-5.68 (m, 1H, CH═CH), 6.34 (d, 1H, J=8.0 Hz, HN), 6.85-6.89 (m, 4H, Ar), 7.26-7.37 (m, 7H, Ar), and 7.49-7.51 (m, 2H, Ar).

Preparation of Compound 46:

Compound 44 (327 mg,0.292 mmol) was dissolved in dry THF (12 mL) and stirred with a solution of 1M sodium methoxide solution (12 mL) at room temperature. TLC (toluene:acetone, 5:1) showed absence of the starting material after 1 hour and was acidified with IR 15 to pH 5-6. The resin was filtered and washed with THF. The solvents from combined filtrate distilled off and the residue was chromatographed. Elution with toluene:acetone(10:1) gave 46 as colorless solid (322 mg, 98%). ¹H NMR (CDCl₃) δ: 0.86 (t, 6H, J=6.5 Hz, 2×CH₃), 1.24 (br s, 58H, CH₂), 1.6 (m, 4H), 1.97-2.1 (m, 4H), 1.97-2.1 (m, 2H), 2.1 (t, 2H, J=7.5 Hz), 3.59 (dd, 1H, J=3.5 & 10.5 Hz) 3.70-3.75 (m,1H), 3.78-3.79 (2s, 6H, 2×OCH₃), 3.86-3.96 (m, 4H), 4.02 (dd, 1H, J=3.5 & 9.5), 4.11-4.18 (m, 3H), 4.61 (d, 1H, J=12.0 Hz), 4.67 (br s, 2H), 4.79-4.82 (m, 2H), 5.38-5.45 (m, 3H), 5.60-5.67 (m, 1H, CH═CH), 6.34 (d, 1H, J=7.5 Hz, NH), 6.83-6.86 (m, 4H, Ar—OCH₃), 7.24-7.37 (m, 7H, Ar), and 7.47-7.5 (m, 2H, Ar).

Preparation of Compound 5:

Trifluoroacetic acid (aq. 95%, 0.5 mL) was added to a solution of 3-hydroxyl blocked compound 27 (120 mg) in dry dichloromethane (9.5 mL) and reaction mixture stirred in dark at room temperature. The reaction was followed by TLC (CHCl₃:MeOH:H₂O, 10:1:0.1) and quenched with few drops of saturated sodium bicarbonate. The reaction mixture was diluted with chloroform and washed with water and organic extract dried over anhydrous sodium sulphate. The solvent was distilled off and residue chromatographed and eluted with chloroform:methanol:water (20:1:0.1) to get the alpha-Gal ceramide analogue 31 (34 mg, 45%). ¹H NMR (CDCl₃+CD₃OD) δ: 0.90 (m, 6H, 2×CH₃), 1.25-1.40 (m, 34H, CH₂), 1.67-1.72 (m, 2H), 2.01-2.15 (m, 6H), 2.21-2.25 (brt, 2H), 2.81-2.86 (m, 6H), 3.59-3.60 (t, 1H), 3.61-3.63 (t, 1H), 3.70-3.83 (m, 11H), 3.89-4.0 (m, 3H), 4.08-4.11 (t, 1H), 4.51-4.53 (br t, 1H), 4.88 (d, 1H, J=3.5 Hz, H-1), 4.97-4.99 (t, 1H), 5.35-5.40 (m, 8H, CH═CH), 5.43-5.48 (m, 1H, CH═CH, Cer), and 5.70-5.77 (m, 1H, CH═CH, Cer).

Preparation of Compound 47:

Compound 46 (226 mg, 0.202 mmol) was dissolved in a mixture of methylene chloride:water (10:1, 22 mL) and stirred with DDQ (138 mg, 0.606 mmol) at room temperature for 4 hours. The reaction mixture was diluted with methylene chloride (80 mL), washed with water (5×30 mL) and dried over anhydrous sodium sulphate. The organic extract was filtered, washed with DCM and solvent from combined filtrate distilled off. The residue was chromatographed and elution with chloroform:methanol (20:1) gave 47 as white solid (150 mg, 80%). ¹H NMR (CDCl₃) δ: 0.95 (t, 6H, J=7.0 Hz, 2×CH₃), 1.22-1.39 (m, 72H, CH₂), 1.59-1.64 (m, 2H), 2.02-2.07 (m, 2H), 2.2 (t, 2H, J=7.5 Hz), 3.69-3.75 (m, 3H), 3.85 (dd, 1H, J=3.5 & 10.5 Hz), 3.89-3.92 (m, 2H), 3.98-4.01 (m, 1H), 4.06-4.08 (dd, 1H, J=2.0 & 12.5 Hz), 4.11-4.14 (t, 1H, J=6.0 Hz), 4.22-4.26 (m, 2H), 4.85 (d, 1H, J=3.0 Hz, H-1), 5.42-5.48 (m, 1H, CH═CH), 5.58 (s, 1H, CHPh), 5.72-5.78 (m, 1H, CH═CH), 7.36-7.39 (m, 3H, Ar), 7.50-7.54 (m, 2H, Ar) and 7.5-7.54 (m, 2H, Ar).

Preparation of Compound 6:

The 4,6-O-benzylidene compound 47 (64 mg) was dissolved in 80% aq acetic acid (6 mL) and heated at 80° C. for 20 hrs. The solvent was distilled off under high vacuum and residue chromatographed. Elution with chloroform:methanol (12:1, with 0.1% water) gave the ∝-Gal-ceramide 6 as colorless product (45 mg, 78%). ¹H NMR (CDCl₃+CD₃OD) δ: (0.89 (t, 6H, J=7.0 Hz, 2×CH₃), 1.25-1.31 (br s, 68H, CH₂), 1.58-1.63 (m, 2H), 1.97-2.06 (m, 2H), 2.18-2.2 (br t, 2H), 3.72-3.83 (m, 6H), 3.95-4.0 (t, 1H,J=7.0 Hz), 4.98 (d,1H, J=3.5 Hz, H-1), 5.43-5.49 (m,1H,CH═CH), and 5.70-5.76 (m, 1H, CH═CH).

Preparation of Compound 7:

A mixture of 47 (55 mg) in THF/MeOH/AcOH (5:5:1, 30 mL) and 10% Pd/C (30 mg) was stirred under hydrogen atmosphere and reaction was monitored by TLC (chloroform/methanol, 8:1). The catalyst was filtered and washed with chloroform/methanol (1:1) and solvent from combined filtrate distilled off. The residue was chromatographed on silica gel and elution with chloroform/methanol/water (10:1:0.1) gave 7 as white solid (27 mg, 68%). ¹H NMR (CDCl₃+CD₃OD, 400 MHz) δ: 0.89 (t, 6H, J=7.0 Hz, 2×CH₃), 1.25 (br s, 68H, CH₂), 1.35 (m, 2H), 1.45 (m, 2H), 1.55 (m, 2H), 2.14 (t, J=7.5 Hz, 2H), 3.44 (m, 2H), 3.60 (m, 1H), 3.64 (dd, J=11.0, 5.0 Hz, 1H), 3.67 (dd, J=11.0, 3.5 Hz, 1H), 3.69 (dd, J=10.5, 2.5 Hz, 1H), 3.70 (m, 1H), 3.73 (dd, J=10.5, 4.0 Hz, 1H), 3.78 (dd, J=10.5, 3.0 Hz, 1H), 3.89 (d, J=4.0 Hz, 1H), 4.80 (d, J=4.0 Hz, 1H). C₅₀ H₉₉NO₈ (841.73); ESI-MS: found 864.7 (M+Na).

Preparation of Compound 50:

HgBr₂ (0.18 g, 0.518 mmol) and Hg(CN)₂ (1.568 g, 6.216 mmol) were dissolved in acetonitrile-benzene (1:1, 22 mL) and the mixture was heated to distill off about 10% of its volume. The mixture was cooled to room temperature and compound 48 (4.26 g, 11.36 mmol), 49 (2.0 g, 5.18 mmol), and calcium sulfate (5.0 g) were added. The mixture was stirred at room temperature for 3 h and dichloromethane (30 mL) was added. The solid was filtered through celite, washed with dichloromethane. The filtrate was washed successively with 30% potassium iodide solution, saturated NaHCO₃ solution and water, and dried over sodium sulfate. After concentration in vacuo, the residue was purified by flash chromatography (ethyl acetate:hexane, 1:5) to give 50 (2.55 g, 69%). R_{f} 0.34 (hexane:ethyl acetate, 3:1). C₄₁H₆₄O₆ (716.43). ¹H NMR (300 MHz, CDCl₃): δ: 0.67 (s, 3H, CH₃), 0.85 (d, J=7.0 Hz, 3H, CH₃), 0.85 (d, J=6.5 Hz, 3H, CH₃), 0.91 (d, J=6.5 Hz, 3H, CH₃), 0.98 (s, 3H, CH₃), 1.00-1.60 (m, 21H), 1.80-1.95 (m, 5H), 1.98 (s, 3H, CH₃CO), 2.04 (s, 3H, CH₃CO), 2.06 (s, 3H, CH₃CO), 2.14 (s, 3H, CH₃CO), 2.18-2.25 (m, 2H), 3.48 (m, 1H, chol-3-H), 3.88 (m, 1H, H-5), 4.10 (dd, J=11.0, 7.0 Hz, 1H, H-6a), 4.18 (dd, J=11.0, 6.5 Hz, 1H), 4.54 (d, J=7.5 Hz, 1H), 5.01 (dd, J=10.5, 3.5 Hz, 1H, H-3), 5.18 (dd, J=10.5, 7.5 Hz, 1H, H-2), 5.37 (m, 2H, H-4, chol-H-6).

Preparation of Compound 8:

Compound 50 (650 mg, 0.908 mmol) was treated with 0.1 M sodium methoxide in methanol (15 mL) at room temperature for 2 h. Add dry chloroform so often as to keep the reaction mixture in a translucent state. When the reaction was complete, add strong acidic resin to neutralize the solution. The resin was filtered off and washed with methanol- dichloromethane (1:1) and the filtrate was concentrated in vacuo. The residue was crystallized from ethyl acetate to afford 8 (411 mg, 83%) as a white solid. R_{f} 0.24 (chloroform:methane, 8:1). ¹H NMR (500 MHz, CDCl₃+CD₃OD): δ: 0.69 (s, 3H, CH₃), 0.85 (d, J=6.5 Hz, 3H, CH₃), 0.85 (d, J=6.5 Hz, 3H, CH₃), 0.92 (d, J=6.5 Hz, 3H, CH₃), 1.01 (s, 3H, CH₃), 1.05-1.65 (m, 21H), 1.80-2.05 (m, 5H), 2.26 (m, 1H), 2.41 (m, 1H), 3.47-3.61 (m, 4H, H-2, H-3, H-5, chol-H-3), 3,76 (d, J=6.0 Hz, 2H, H-6a, H-6b), 3.91 (d, J=2.0 Hz, 1 H, H-4), 4.34 (d, J=7.0 Hz, 1H, H-1), 5.37 (br s, 1H, chol-H-6). C₃₃H₅₆O₆ (548.42). ESIMS found: 571.4 (M+Na).

Preparation of Compounds 51α and 51β:

A mixture of compound 29 (1.82 g, 2.79 mmol), 49 (400 mg, 0.776 mmol) and molecular sieves (3 Å, 0.5 g) in dry tetrahydrofuran (15 mL) was stirred under nitrogen for 15 min. The reaction flask was cooled to −20° C. and trimethylsilyl trifluoromethanesulfonate solution (TMSOTf, 0.01 Main CH₂Cl₂, 2.33 mL) was added drop wise to the reaction mixture. The mixture was stirred at −20° C. for 1 h and the reaction quenched by the addition of triethylamine ((0.2 mL). The solid was filtered out and the filtrate concentrated. The residue was purified by flash chromatography (hexane:ethyl acetate, 9:1 and 6:1) to give 51a (316 mg, 28%) and 51 b (774 mg, 68%).

For 51α: R_{f} 0.61 (hexane:ethyl acetate, 6:1); ¹H NMR (500 MHz, CDCl3): δ: 0.68 (s, 3H, CH₃), 0.86 (d, J=6.5 Hz, 3H, CH₃), 0.86 (d, J=6.5 Hz, 3H, CH₃), 0.92 (d, J=6.5 Hz, 3H, CH₃), 1.01 (s, 3H, CH₃), 1.04-1.64 (m, 21H), 1.80-2.04 (m, 5H), 2.23 (m, 1H), 2.40 (m, 1H), 3.45 (m, 1H, chol-H-3), 3.69 (br s, 1H), 3.80 (s, 6H, 2OCH₃), 3.96 (dd, J=10.0, 3.5 Hz, 1H), 4.00 (dd, J=12.0, 2.0 Hz, 1H, H-6a), 4.01 (dd, J=10.0, 3.5 Hz, 1H), 4.15 (d, J=3.5 Hz, 1H, H-4), 4.19 (dd, J=12.0, 2.0 Hz, 1H, H-6b), 4.58 (d, j=11.5 Hz, 1H, CHHPh), 4.65 (d, J=11.5 Hz, 1H, CHHPh), 4.75 (d, J=11.5 Hz, 1H, CHHPh), 4.76 (d, J=11.5 Hz, 1H, CHHPh), 5.03 (d, J=3.5 Hz, 1H, H-1), 5.31 (m, 1H, chol-H-6), 5.50 (s, 1H, CHPh), 6.85 (m, 4H), 7.30 (m, 7H), 7.50 (m, 2H). C₅₆H₇₆O₈ (876.55); ESIMS found: 899.5 (M+Na).

For 51β: R_{f} 0.50 (hexane:ethyl acetate, 6:1); ¹H NMR (300 MHz, CDCl₃): δ: 0.68 (s, 3H, CH₃), 0.86 (d, J=6.5 Hz, 3H, CH₃), 0.86 (d, J=6.5 Hz, 3H, CH₃), 0.92 (d, J=6.5 Hz, 3H, CH₃), 1.03 (s, 3H, CH₃), 1.05-1.60 (m, 21H), 1.80-2.05 (m, 5H), 2.28-2.45 (m, 2H), 3.30 (br s, 1H, H-5), 3.52 (dd, J=10.0, 3.5 Hz, 1H), 3.59 (m, 1H, chol-H-5), 3.75 (m, 1H), 4.10 (dd, J=12.0, 2.0 Hz, 1H, H-6a), 4.05 (d, J=3.5 Hz, 1H, H-4), 4.25 (d, J=12.0, 2.0. Hz, 1H, H-6b), 4.49 (d, J=8.0 Hz, 1H, H-1), 4.67 (d, J=12.0 Hz, 1H, CHHPh), 4.67 (d, J=10.5 Hz, 1H, CHHPh), 4.73 (d, J=12.0 Hz, 1H, CHHPh), 4.87 (d, J=10.5 Hz, 1H, CHHPh), 5.34 (m, 1H, chol-H-6), 5.50 (s, 1H, CHPh), 6.85 (m, 4H), 7.30 (m, 7H), 7.55 (m, 2H). ESIMS found: 894.5 (M+NH4), 899.5 (M+Na), 915.5 (M+K).

Preparation of Compound 52:

Compound 51α (289 mg, 0.33 mmol) was dissolved in dichloromethane-water (10:1, 30 mL) and DDQ (224 mg, 0.99 mmol) was added. The mixture was stirred at room temperature for 3 h and diluted with dichloromethane (100 mL). The mixture was washed with saturated sodium bicarbonate solution (50 mL) and water (50 mL), and the organic layer dried over sodium sulfate, concentrated. The residue was purified by flash chromatography (hexane:ethyl acetate, 1:1) to give 52 (190 mg, 90%). ¹H NMR (300 MHz, CDCl₃): δ: 0.68 (s, 3H, CH₃), 0.86 (d, J=6.5 Hz, 3H, CH₃), 0.86 (d, J=6.5 Hz, 3H, CH₃); 0.92 (d, J=6.5 Hz, 3H, CH₃), 1.01 (s, 3H, CH₃), 1.04-1.62 (m, 21H), 1.78-2.04 (m, 5H), 2.35 (m, 2H), 3.52 (m, 1H), 3.64 (m,2H), 3.74 (m, 1H), 3.80 (br s, 1H, H-5), 3.88 (br s, 2H, 2 OH), 4.08 (dd, J=12.0, 1.5 Hz, 1H, H-6a), 4.27 (br s, 1H, H-4), 4.28 (dd, J=12.0, 1.5 Hz, 1H, H-6b), 5.19 (br s, 1H, H-1), 5.36 (m, 1H, chol-H-6), 5.56 (s, 1H, CHPh), 7.37 (m, 3H), 7.50 (m, 2H). C₄₀H₆₂O₆ (638.9).

Preparation of Compound 9:

The suspension of compound 52 (179 mg, 0.28 mmol) in acetic acid-water (4:1, 5 mL) was treated at 80° C. for 2 h. the mixture was then cooled to room temperature and concentrated in vacuo. The residue was purified by flash chromatography (chloroform:methanol:water (10:1:0.1) to give 9 (108 mg, 70%). R_{f} 0.22 chromatography (chloroform:methanol:water (10:1:0.1). ¹H NMR (600 MHz, CDCl₃+CD₃OD+D₂O): δ: 0.68 (s, 3H, CH₃), 0.87 (d, J=6.5 Hz, 3H, CH₃), 0.87 (d, J=6.5 Hz, 3H, CH₃), 0.93 (d, J=6.5. Hz, 3H, CH₃), 1.02 (s, 3H, CH₃), 1.05-1.62 (m, 21H), 1.81-2.04 (m, 5H), 2.36 (m, 2H), 3.50 (m, 1H, chol-5-H), 3.72-3.77 (m, 4H), 3.92 (dd, J=6.0, 6.0 Hz, 1H, H-5), 3.98 (br s, 1H, H-4), 5.02 (d, J=3.5 Hz, 1H, H-1), 5.35 (m, 1H, chol-H-6). C₃₃H₅₆O₆ (548.42); ESIMS found: 571.4 (M+Na).

Preparation of Compound 54:

HgBr₂ (175 mg, 0.48 mmol and HgCN₂ (1.47 g, 5.83 mmol) were dissolved in acetonitrile-benzene (1:1, 22 mL) and the mixture were refluxed to distill off about 10% of the total volume. The solution was cooled to room temperature and acetobromogalactose 48 (3.99 g, 9.71 mmol), stigmasterol 53 (2.0 g, 4.85 mmol) and CaSO₄ (5.0 g) were added. The mixture was stirred at room temperature overnight and then diluted with dichloromethane (100 mL), the solid was filtered and the filtrate was washed successively with 30% potassium iodide solution, saturated sodium bicarbonate solution, and water. The organic layer was dried over sodium sulfate and concentrated. The residue was purified by flash chromatography to give 54 (2.49 g, 72%).

Preparation of Compound 10:

Compound 54 (2.37 g, 3.19 mmol) was dissolved in the solution of 0.1 M sodium methoxide in methanol and dry chloroform was added to keep the solution translucent. The mixture was stirred under nitrogen for 2 h and strong acidic resin IR-120 was, added to neutralize the solution. The resin was filtered and washed with chloroform-methanol (1:1) and the filtrate concentrated in vacuo. The residue was crystallized from ethyl acetate to give 10 (1:07 g, 58%) as white solid. ¹H NMR (400 MHz, CDCl₃+CD₃OD): δ: 0.71 (s, 3H, CH₃), 0.80 (d, J=6.5 Hz, 3H, CH₃), 0.81 (t, J=6.5 Hz, 3H, CH₃), 0.86 (d, J=6.5 Hz, 3H, CH₃), 0.93 (m, 1H), 1.01 (s, 3H, CH₃), 1.03 (d, J=6.5 Hz, 3H, CH₃), 1.05-1.73 (m, 17H), 1.83-2.08 (m, 5H), 2.25 (m, 1H), 2.40 (m, 1H), 3.48 (m, 3H), 3.58 (m, 1H, chol-H-3), 3.74 (m, 2H), 3.89 (m, 1H), 5.02 (dd, J=15.5, 9.0 Hz, 1H), 5.16 (dd, J=15.5, 8.5 Hz, 1H), 5.34 (m, 1H, chol-H-6). C₃₅H₅₈O₆ (574.43); ESIMS found: 597.4 (M+Na).

Preparation of Compound 55:

To the mixture of compound 53 (400 mg, 0.97 mmol) and molecular sieve (4 Å, 0.5 g) in dry tetrahydrofuran (2.0 mL) was added trimethylsilyl trifluoromethanesulfonate solution (0.01 M in THF, 9.7 mL) drop wise. A solution of compound 29 (2.00 g, 2.91 mmol) in dry THF (5.0 mL) was added to the reaction mixture drop wise, which was stirred at room temperature for 1.5 h. the reaction was quenched by adding triethylamine (0.2 mL) and the solid was filtered off. The filtrate was concentrated in vacuo and the residue was purified by flash chromatography (hexane:ethyl acetate, 5:1) to give the desired α-glycoside (27.1 mg, 31%). ¹H NMR (300 MHz, CDCl₃): δ: 0.70 (s, 3H, CH₃), 0.80 (d, J=6.5 Hz, 3H, CH₃), 0.81 (t, J=6.5 Hz, 3H, CH₃), 0.85 (d, J=6.5 Hz, 3H, CH₃), 0.92 (m, 1H), 1.02 (s, 3H, CH₃), 1.03 (d, J=6.5 Hz, 3H, CH₃), 1.10-1.70 (m, 17H), 1.85-2.08 (m, 5H), 2.23 (m, 1H), 2.40 (m, 1H), 3.45 (m, 1H, chol-H-3), 3.75 (br s, 1H), 3.80 (s, 6H, 2OCH₃), 4.00 (m, 3H), 4.20 (m, 2H), 4.58 (d, J=12.0 Hz, 1H, CHHPh), 4.65 (d, J=12.0 Hz, 1H), 4.76 (d, 12.0 Hz, 1H, CHHPh), 4.77 (d, J=12.0 Hz, 1H, CHHPh), 5.00 (m, 1H), 5.05 (d, J=3.5 Hz, 1H, H-1), 5.15 (dd, J=12.0, 8.5 Hz, 1H), 5.32 (m, 1H, chol-H-3), 5.45 (s, 1H, CHPh), 6.85 (m, 4H), 7.30 (m, 7H), 7.52 (m, 2H). C₅₈H₇₈O₈ (903.20).

Preparation of Compound 11:

Compound 55 (50 mg, 0.055 mmol) was dissolved in dichloromethane-water (10:1, 1 mL) and DDQ (50 mg, 0.22 mmol) was added. The mixture was stirred at room temperature for 6 h and then diluted with dichloromethane (20 mL), the mixture was washed with sat. sodium bicarbonate solution (10 mL) and water (10 mL) and organic layer was dried over sodium sulfate and concentrated. The residue was purified by flash chromatography (hexane:ethyl acetate, 2:1) to the p-methoxybenzyl-removed product (28 mg, 76%).

The p-methoxybenzyl-deprotected material (70 mg, 0.106 mmol) was dissolved in HOAc-water (4:1, 5 mL) and the solution was treated at 80° C. for 16 h. the solvent was removed and the residue was crystallized from ethyl acetate to give 11 (38 mg, 63%). ¹H NMR (500 MHz, CD₃OD): δ: 0.71 (s, 3H, CH₃), 0.79 (d, J=6.5 Hz, 3H, CH₃), 0.80 (t, J=6.5 Hz, 3H, CH₃), 0.85 (d, J=6.5 Hz, 3H, CH₃), 0.93 (m, 1H), 1.01 (s, 3H, CH₃), 1.02 (d, J=6.5 Hz, 3H, CH₃), 1.13-1.58 (m, 16H), 1.70 (m, 1H), 1.85-2.08 (m, 5H), 2.35 (m, 2H), 2.61 (m, 1H), 3.48 (m, 1H), 3.75 (m, 4H), 3.89 (m, 1H), 3.98 (br s, 1H), 5.03 (m, 2H), 5.16 (dd, J=15.0, 9.0 Hz, 1H), 5.34 (m, 1H). C₃₅H₅₈O₆ (574.42). ESIMS found: 597.4 (M+Na).

Preparation of Compounds 57α and 57β:

A mixture of compound 29 (400 mg, 0.613 mmol), β-sitosterol 56 (100 mg, 0.241 mol) and molecular sieve (3 Å, 0.5 g) in dry THF (5 mL) was stirred at room temperature for 5 min. the reaction flask was cooled to −20° C. and trimethylsilyl trifluoromethanesulfonate solution (0.01 M in dichloromethane, 0.72 mL) was added drop wise. The reaction mixture was stirred at −20° C. for 1 h and then triethylamine (0.1 mL) was added to quench the reaction. The solid was filtered off and the filtrate concentrated in vacuo. The residue was purified by flash chromatography (hexane:ethyl acetate, 6:1) to give 57α (58 mg, 27%) and 57β (95 mg, 44%).

For 57α: R_{f} 0.56 (hexane:ethyl acetate, 3:1); ¹H NMR (400 MHz, CDCl₃): δ: 0.68 (s, 3H, CH₃), 0.81 (d, J=6.5 Hz, 3H, CH₃), 0.83 (d, J=6.5 Hz, 3H, CH₃), 0.85 (t, J=6.5 Hz, 3H, CH₃), 0.93 (d, J=6.5 Hz, 3H, CH₃), 1.01 (s, 3H, CH₃), 1.02-1.70 (m, 22H), 1.80-2.04 (m, 5H), 2.23 (m, 1H), 2.40 (m, 1H), 3.45 (m, 1H), 3.68 (br s, 1H, H-5), 3.80 (s, 6H, 2OCH₃), 3.95 (dd, J=10.0, 3.5 Hz, 1H), 4.00 (dd, J=12.0, 2.0 Hz, 1H, H-6a), 4.01 (dd, J=10.0, 3.5 Hz, 1H), 4.15 (d, J=3.5 Hz, 1H), 4.19 (dd, J=12.0, 1.5 Hz, 1H, H-6b), 4.58-(d, J=11.5 Hz, 1H, CHHPh), 4.65 (d, J=11.5 Hz, 1H, CHHPh), 4.75 (d, J=11.5 Hz, 1H, CHHPh), 4.76 (d, J=11.5 Hz, 1H, CHHPh), 5.03 (d, J=3.5 Hz, 1H, H-1), 5.32 (m, 1H), 5.46 (s, 1H, CHPh), 6.85 (m, 4H), 7.30 (m, 7H), 7.50 (m, 2H). C₅₈H80O₈ (904.59). ESIMS found: 927.6 (M+Na)

For 57β: R_{f} 0.42 (hexane:ethyl acetate, 3:1); ¹H NMR (400 MHz, CDCl₃): δ: 0.68 (s, 3H, CH₃), 0.81 (d, J=6.5 Hz, 3H, CH₃), 0.83 (d, J=6.5 Hz, 3H, CH₃), 0.85 (t, J=6.5 Hz, 3H, CH₃), 0.93 (d, J=6.5 Hz, 3H, CH₃), 1.03 (s, 3H, CH₃), 1.04-1.73 (m, 22H), 1.80-2.05 (m, 5H), 2.33 (m, 1H), 2.42 (m, 1H), 3.28 (br s, 1H, H-5), 3.50 (dd, J=10.0, 4.0 Hz, 1H, H-3), 3.59 (m, 1H), 3.77 (dd, J=10.0, 8.0 Hz, 1H, H-2), 3.80 (s, 6H, 2OCH₃), 3.99 (dd, J=12.0, 2.0 Hz, 1H, H-6a), 4.04 (d, J=4.0 Hz, 1H, H-4), 4.26 (dd, J=12.0, 1.5 Hz, 1H, H-6b), 4.48 (d, J=8.0 Hz, 1H, H-1), 4.65 (d, J=12.0 Hz, 1H, CHHPh), 4.68 (d, J=11.5 Hz, 1H, CHHPh), 4.72 (d, J=12.0 Hz, 1H, CHHPh), 4.87 (d, J=11.5 Hz, 1H, CHHPh), 5.33 (m, 1H), 5.48 (s, CHPh), 6.85 (m, 14H), 7.30 (m, 7H), 7.50 (m, 2H). C₈H₈₀O₈ (904.59). ESIMS found: 927.6 (M+Na).

Preparation of Compound 58:

Compound 57β (82 mg, 0.091 mmol) was dissolved in dichloromethane-water (10:1, 5.5 mL) and DDQ (62 mg, 0.273 mmol) was added. The mixture was stirred at room temperature for 3 h and then diluted with dichloromethane (30 mL), the organic layer was washed with sat. sodium bicarbonate solution (15 mL) and water (15 mL) and aqueous layer extracted with chloroform (3×30 mL), the combined organic layer was dried over sodium sulfate and concentrated. The residue was purified by flash chromatography (hexane:ethyl acetate:methanol, 10:10:0.5) to give 58 (44 mg, 73%). ¹H NMR (300 MHz, CDCl₃): δ: 0.68 (s, 3H, CH₃), 0.81 (d, J=6.5 Hz, 3H, CH₃), 0.83 (d, J=6.5 Hz, 3H, CH₃), 0.85 (t, J=6.5 Hz, 3H, CH₃), 0.92 (d, J=6.5 Hz, 3H, CH₃), 1.02 (s, 3H, CH₃), 1.05-1.73 (m, 22H), 1.80-2.05 (m, 6H), 2.30 (m, 1H), 2.45 (m, 2H), 3.47 (br s, 1H, H-5), 3.60-3.77 (m, 4H), 4.08 (dd, J=12.0, 2.0 Hz, 1H, H-6a), 4.21 (d, J=3.5 Hz, 1H, H-4), 4.32 (dd, J=12.0, 1.0 Hz, 1H, H-6b), 4.40 (d, J=7.5 Hz, 1H, 5.36 (m, 1H), 5.50 (s, 1H, CHPh), 7.35 (m, 3H), 7.50 (m, 2H). C₄₂H₆₄O₆ (664.32). ESIMS found: 687.3 (M+Na).

Preparation of Compound 12:

Compound 58 (11 mg, 0.017 mmol) was dissolved in acetic acid-water (4:1, 5 mL) and treated at 80° C. for 2 h. the solvent was removed and the residue was purified by flashed chromatography (chloroform:methanol:water, 10:1: 0.1) to give 12 (7 mg, 73%). ¹H NMR (300 MHz, CDCl₃+CD₃OD+D₂O): δ: 0.72 (s, 3H, CH₃), 0.84 (d, J=6.5 Hz, 3H, CH₃), 0.86 (d, J=6.5 Hz, 3H, CH₃), 0.87 (t, J=6.5 Hz, 3H, CH₃), 0.95 (d, J=6.5H, 3H, CH₃), 1.04 (s, 3H, CH₃), 1.05-1.73 (m, 22H), 1.80-2.05 (m, 5H), 2.28 (m, 1H), 2.43 (m, 1H), 3.50 (dd, J=10.0, 7.5 Hz, 1H, H-2), 3.53 (m, 2H), 3.62 (mk, 1H), 3.75 (m, 2H), 3.91 (d, J=3.5 Hz, 1H, H-4), 4.37 (d, J=7.5 Hz, 1H, H-1), 5.39 (m, 1H). C₃₅H₆₀O₆ (576.41). ESIMS found: 599.4 (M+Na).

Preparation of Compound 59:

Compound 57α (48 mg, 0.053 mmol) was dissolved in dichloromethane-water (10:1, 5.5 mL) and DDQ (36 mg, 0.159 mmol) was added. The mixture was stirred at room temperature for 3 h and then diluted with dichloromethane (50 mL). The organic layer was washed with sat. sodium bicarbonate solution (20 mL) and water (20 mL), and dried over sodium sulfate and concentrated. The residue was purified by flash chromatography (hexane:ethyl acetate, 1:1) to give 59 (27 mg, 77%). %). ¹H NMR (300 MHz, CDCl₃): δ: 0.68 (s, 3H, CH₃), 0.81 (d, J=6.5 Hz, 3H, CH₃), 0.84 (d, J=6.5 Hz, 3H, CH₃), 0.85 (t, J=6.5 Hz, 3H, CH₃), 0.92 (d, J=6.5 Hz, 3H, CH₃), 1.02 (s, 3H, CH₃), 1.05-1.73 (m, 22H), 1.80-2.05 (m, 5H), 2.35 (m, 2H), 3.54 (m, 1H), 3.75 (m, 1H), 3.80 (r s, 1H, H-5), 3.89 (m, 1H), 4.10 (dd, J=12.0, 2.0 Hz, 1H, H-6a), 4.28 (br s, 1H, H-4), 4.28 (dd, J=12.0, 1.5 Hz, 1H, H-6b), 5.19 (d, J=3.5 Hz, 1H, H-1), 5.35 (m, 1H), 5.56 (s, 1H, CHPh), 7.36 (m, 3H), 7.50 (m, 2H). C₄₂H₆₄O₆ (664.32). ESIMS found: 687.4 (M+Na).

Preparation of Compound 13:

Compound 59 (25 mg, 0.038 mmol) was dissolved in acetic acid-water (4:1, 10 mL) and treated at 80° C. for 2 h. The solvent was removed and the residue was purified by flashed chromatography (chloroform:methanol:water, 10:1:0.1) to give 13 (1.3 mg, 60%). ¹H NMR (300 MHz, CDCl₃+CD₃OD+D₂O): δ: 0.73 (s, 3H, CH₃), 0.82 (d, J=6.5 Hz, 3H, CH₃), 0.84 (d, J=6.5 Hz, 3H, CH₃), 0.86 (t, J=6.5 Hz, 3H, CH₃), 0.94 (d, J=6.5 Hz, 3H, CH₃), 1.02 (s, 3H, CH₃), 1.05-1.73 (m, 22H), 1.80-2.05 (m, 5H), 2.35 (m, 2H), 3.48 (m, 1H), 3.75 (m, 4H), 3.91 (m, 1H), 3.98 (br s, 1H, H-4), 5.01 (br s, 1H), 5.35 (m, 1H). C₃₅H₆₀O₆ (576.41). ESIMS found: 599.4 (M+Na).

Common Abbreviations Used in the Document

All allyl
APC antigen presenting cell
BF₃OEt₂ trifluoroboran diethyl etherate
Bn benzyl
Bz benzoyl
^{t}Bu tert-butyl
m-CPBA m-chloroperbenzoic acid
CPM counts per miniute
DBU 1,8-diazabicyclo[5,4,0]undec-7-ene
DCC dicyclohexylcarbodiimide
(-)-DIPCl (-)-B-Chlorodiisopinocamphenylborane
DMAP 4-dimethylaminopyridine
DMF dimethylformamide
DMPC dimyristoyl phosphatidyl glycerol
DPPC dipalmitoyl phosphatidyl choline
DMSO dimethyl sulfoxide
EDCI 1-[3-(dimethylamino)propyl]-3-ethylcarbodiimide hydrochloride
ES-MS electron spray mass spectrometry
Et ethyl
Fmoc 9-fluorenylmethoxylcarbonyl
IFN-γ interferon-gamma
IL interleukin
LPS lipopolysaccharide
Me methyl
MLV multilamellar large vesicles
NBS N-bromosuccinimide
NMM N-methyl morpholine
NMR nuclear magnetic resonance
Pal palmitoyl
Ph phenyl
Phth phthalimido
PMB para-methoxylbenzyl
iPr isopropyl
Py pyridine
SUV small unilamellar vesicles
Tf trifluoromethylsulfonyl
TFA trifluoroacetic acid
THF tetrahydrofuran
TLC thin layer chromatography
Troc trichloroethoxylcarbonyl
Trt triphenylmethyl
p-TsOH p-toluenesulfonic acid

### REFERENCES CITED

Natori, T.; Morita, M.; Akimoto, K.; Koezuka, Y. Tetrahedron, 1994, 50, 2771-2784.
Kawano, T. Cui, J.; Koezuka, Y.; Toura, I.; Kaneko, Y.; Motoki, K.; Ueno, H.; Nakagawa, R.; Sato, H.; Kondo, E. et al. Science, 1997, 278, 1626-1629.
Gonzalez-Aseguinolaza, G.; Kaer, L. V.; Bergmann, C. C.; Wilson, J. M.; Schmieg, J.; Kronenberg, M.; Nakayama, T.; Taniguchi, M.; Koezuka, Y.; Tsuji, M. J. Exp. Med. 2002, 195, 617-624.
Miyamoto, et al., “A synthetic glycolipid prevents autoimmune encephalomyelitis by inducing Th2 bias of natural killer T cells,” Nature, 413: 531 (Oct. 4, 2001)
Brossay, et al., J. Immunol., 16: 5124-28 (1998)
Park, S.-H. & Bendelac, A. Nature, 2000, 406, 788-792
Uchimura, A. et al. Bioorg. Med. Chem. 1997, 5, 1447
Uchimura, A. et al. Bioorg. Med. Chem. 1997, 5, 2245-2249
Costantino, V. et al. Tetrahedron, 1996, 52, 1573-1578
Morita, M. et al. J. Med. Chem. 1995, 38, 2176-2187.
Kawano et al., Science, 1997, 278, 1616-1629)
Costantino, et al., Bioorgan. Med. Chem. Lett. 9: 271-6 (1999)
Marsh, E. Neil G., “Toward the nonstick egg: designing fluorous proteins”, Chemistry & Biology 7:R153-R157 (2000). Indeed, fluorination can increase binding; trifluoroleucine syubstitution in melittin had enhanced affinity for lipid bilayer membranes. Niemz and Tirrell, “Self-association and membrane-binding behavior of melittins containing trifluoroleucine”, J. Am. Chem. Soc. 123: 7407-13 (2001).
Faroux-Corlay, et al., “Synthesis of single- and double-chain fluorocarbon and hydrocarbon galactosyl amphiphiles and their anti-HIV-1 activity”, Carbohydr. Res., 327: 223-260 (2000)
Faroux-Corlay. et al., “Amphiphilic anionic analogues of galactosylceramide: synthesis, anti-HIV-1 activity, and gp120 binding,” J. Med. Chem., 44: 2188-2203 (2001)
Clary, et al., “Synthesis of single- and double-chain fluorcarbon and hydrocarbon β-linked galactose amphiphiles derived from serine,” Tetrahedron Lett., 36: 539-42 (1995).
Motoki, U.S. Pat. No. 6,555,372
Taniguchi, U.S. Pat. No. 6,531,453
Longwood, U.S. Pat. No. 6,103,883
Shayman, U.S. Pat. No. 6,569,889
Maruyama, U.S. Pat. No. 6,417,167.
Lindhorst, et al, Eur. J. Org. Chem., 2027-34 (2000)
Schmidt, et al. Eur. J. Org. Chem., 669-674 (2002)
Aguilera, Begona; Romero-Ramírez, Lorenzo; Abad-Rodríguez, José; Corrales, Guillermo; Nieto-Sampedro, Manuel; Fermández-Mayoralas, Alfonso. J. Med. Chem. 1998, 41, 4599-4606.
Armspach, Dominique; Cattalini, Marco; Constable, Edwin C.; Housecroft, Catherine E.; Phillips, David. Boron-rich Metallodendrimers—Mix-and-match Assembly of Multifunctional Metallosuperomolecules, Chem. Commun. 1996, 1823-1824.
Cheng, Xiao Hong; Diele, Siegmar; Tschierske, Carsten. Molecular. Design of Liquid-Crystalline Block Molecules: Semifluorinated Pentaerythritol Tetrabenzoates Exhibiting Lamellar, Columnar, and Cubic Mesophases, Angew. Chem. Int. Ed. 2000, 39, 592-595.
Toefper, Alexander; Kretzschmar, Gerhard; Bartnik, Eckart. Tetrahedron Lett. 1995, 36, 9161-9164.
Toefper, Alexander; Kretzschmar, Gerhard; Bartnik, Eckart; Seiffge, Dirk. U.S. Pat. No. 6,136,790, 2000.
Wörl, Ralf; Köster, Hubert. Synthesis of New Liquid Phase Carriers for Use in Large Scale Oligonucleotide Synthesis in Solution, Tetrahedron, 1999, 2941-2956.
Kuzdzal, Scott A.; Monnig, Curtis A.; Newkome, George R.; Moorefield, Charles N. Dendrimer Electrokinetic Capillary Chromatography: Unimolecular Micellar Behaviour of Carbobxylic Acid Terminated Cascade Macromolecules, J. Chem. Soc., Chem. Commun. 1994, 2139-2140.
Hanessian et al. 1996, Synthesis of chemically and functionally diverse scaffolds from pentaerythritol, Can. J. Chem, 74, 1731-1737.
Morita et al. 1995, Structure-activity relationship of a-galactosylceramides against B16-bearing mice, J. Med. Chem. 38, 2176-2187.
Sharif et al. 2001, Activation of natural killer T cells by a-galactosylceramide treatment prevents the onset and recurrence of autoimmune type 1 diabetes, Nat. Med. 7, 1057-1062.
Gonzalez-Aseguinolaza et al. 2000, a-Galactosylceramide-activated Val4 natural killer T cells mediate protection against murine malaria, PNAS, 97, 8461-8466.

Citation of documents herein is not intended as an admission that any of the documents cited herein is pertinent prior art, or an admission that the cited documents is considered material to the patentability of any of the claims of the present application. All statements as to the date or representation as to the contents of these documents is based on the information available to the applicant and does not constitute any admission as to the correctness of the dates or contents of these documents.

The appended claims are to be treated as a non-limiting recitation of preferred embodiments.

In addition to those set forth elsewhere, the following references are hereby incorporated by reference, in their most recent editions as of the time of filing of this application: Kay, Phage Display of Peptides and Proteins: A Laboratory Manual; the John Wiley and Sons Current Protocols series, including. Ausubel, Current Protocols in Molecular Biology; Coligan, Current, Protocols in Protein Science; Coligan, Current Protocols in Immunology; Current Protocols in Human Genetics; Current Protocols in Cytometry; Current Protocols in Pharmacology; Current Protocols in Neuroscience; Current Protocols in Cell Biology; Current Protocols in Toxicology; Current Protocols in Field Analytical Chemistry; Current Protocols in Nucleic Acid Chemistry; and Current Protocols in Human Genetics; and the following Cold Spring Harbor Laboratory publications: Sambrook, Molecular Cloning: A Laboratory Manual; Harlow, Antibodies: A Laboratory Manual; Manipulating the Mouse Embryo: A Laboratory Manual; Methods in Yeast Genetics: A Cold Spring Harbor Laboratory Course Manual; Drosophila Protocols; Imaging Neurons: A Laboratory Manual; Early Development of Xenopus laevis: A Laboratory Manual; Using Antibodies: A Laboratory Manual; At the Bench: A Laboratory Navigator; Cells: A Laboratory Manual; Methods in Yeast Genetics: A Laboratory Course Manual; Discovering Neurons: The Experimental Basis of Neuroscience; Genome Analysis: A Laboratory Manual Series; Laboratory DNA Science; Strategies for Protein Purification and Characterization: A Laboratory Course Manual; Genetic Analysis of Pathogenic Bacteria: A Laboratory Manual; PCR Primer: A Laboratory Manual; Methods in Plant Molecular Biology: A Laboratory Course Manual; Manipulating the Mouse Embryo: A Laboratory Manual; Molecular Probes of the Nervous System; Experiments with Fission Yeast: A Laboratory Course Manual; A Short Course in Bacterial Genetics: A Laboratory Manual and Handbook for Escherichia coli and Related Bacteria; DNA Science: A First Course in Recombinant DNA Technology; Methods in Yeast: Genetics: A Laboratory Course Manual; Molecular Biology of Plants: A Laboratory Course Manual.

All references cited herein, including journal articles or abstracts, published, corresponding prior or otherwise, related U.S. or foreign patent applications, issued U.S. or foreign patents, or any other references, are entirely incorporated by reference herein, including all data, tables, figures, and, text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiments of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Any description of a class or range as being useful or preferred in the practice of the invention shall be deemed a description of any subclass (e.g., a disclosed class with one or more disclosed members omitted) or subrange contained therein, as well as a separate description of each individual member or value in said class or range.

The description of a minimum and the separate description of a maximum, where the maximum is greater than the minimum, imply that in a preferred embodiment the two may be combined to form a fully close-ended range. If the maximum equals the mininimum, a preferred value is implied.

The description of preferred embodiments individually shall be deemed a description of any possible combination of such preferred embodiments, except for combinations which are impossible (e.g, mutually exclusive choices for an element of the invention) or which are expressly excluded by this specification.

The term “comprising”, as used in the claims herein, means that the elements subsequently recited are required, but that the inclusion of additional elements is allowed if not expressly excluded by some other limitation.

The word “a”, unless otherwise qualified, implies “one or more”.

If an embodiment of this invention is disclosed in the prior art, the description of the invention shall be deemed to include the invention as herein disclosed with such embodiment excised.

The invention, as contemplated by applicant(s), includes but is not limited to the subject matter set forth in the appended claims, and presently unclaimed combinations thereof. It further includes such subject matter further limited, if not already such, to that which overcomes one or more of the disclosed deficiencies in the prior art. To the extent that any claims encroach on subject matter disclosed or suggested by the prior art, applicant(s) contemplate the invention(s) corresponding to such claims with the encroaching subject matter excised.

All references, including patents, patent applications, books, articles, and online sources, cited anywhere in this specification are hereby incorporated by reference, as are any references cited by said references.

1. A non-naturally occurring, biologically active compound having the formula F-A 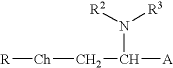
where
R is an organic moiety comprising at least one carbohydrate moiety and/or at least one Pet (pentaerythritol) unit;
Ch is chalcogen;
R2 is hydrogen, or an organic moiety consisting of at least one primarily alkyl moiety and, optionally, one or more spacers;
R3 is —CH2-R3′ or —C(=Ch)-R3′, where R3′ is an organic moiety comprising a steroid moiety, an alkaloid moiety, a terpenoid moiety, a polyunsaturated moiety or a primarily alkyl moiety, and
A is an organic moiety consisting of at least one primarily alkyl moiety and, optionally, one or more spacers; and
at least one of the following conditions applies:
(1) said compound comprises at least one steroid moiety, and/or at least one alkaloid moiety;
(2) R3′ comprises at least one polyunsaturated moiety;
(3) R3′ is of the form -(linker)(-spacer-T^{a})ₐ(-T^{b})_{b}, where linker is an aliphatic moiety with not more than 12 non-hydrogen atoms, and consisting of one or more alkyl moieties and/or one or more spacers, a and b are integers each in the range of 0-3, and a+b is in the range of 1-3, except that if a=0, b is at least 2, and T^{a} and T^{b} are, independently, organic moieties consisting of at least one primarily alkyl moiety and, optionally, one or more spacers, which may differ for each of the a instances of T^{a} and each of the b instances of T^{b};
(4) A is —CH(-spacer-R4)-R1 where
(A) R1 is hydrogen, and R4 is hydrogen or an organic moiety consisting of at least one primarily alkyl moiety and, optionally, one or more spacers;
(B) R1 is an organic moiety consisting of at least one primarily alkyl moiety and, optionally, one or more spacers, and R4 is an organic moiety consisting of at least one primarily alkyl moiety and, optionally, one or more spacers;
(C) R1 is -(spacer cluster)-(organic moiety) and R4 is hydrogen, -(organic moiety), or -(spacer)-(organic moiety), where each organic moiety is one consisting of at least one primarily alkyl moiety and, optionally, one or more spacers; and
(5) A is -(spacer cluster)-R1, where R1 is hydrogen or an organic moiety consisting of at least one primarily alkyl moiety and, optionally, one or more spacers.

2. The compound of claim 1 where each of the organic moieties consists of not more than 120 atoms other than hydrogen atoms.

3. The compound of claim 1 where each chalcogen is oxygen.

4. The compound of claim 1 in which R2 is hydrogen.

5. The compound of claim 1 in which R3 comprises at least one strongly lipophilic group.

6. The compound of claim 1 in which “A” comprises at least one strongly lipophilic group.

7. The compound of claim 1 where condition (1) applies.

8. The compound of claim 7 where R3′ comprises a steroid or alkaloid moiety.

9. The compound of claim 7 where R3′ comprises a steroid moiety.

10. The compound of claim 1 where condition (2) applies.

11. The compound of claim 10 where the polyunsaturated moiety comprises at least one methylene-interrupted pair of alkenic double bonds (—C═C—C—C═C—).

12. The compound of claim 11 where the carbon skeleton of R3 is the same as the carbon skeleton of the fatty acyl moiety of arachidonic acid.

13. The compound of claim 1 in which condition (3) applies.

14. The compound of claim 13 in which each T^{a} and T^{b} is an independently chosen primarily alkyl moiety.

15. The compound of claim 14 in which b=0.

16. The compound of claim 14 in which the linker is divalent.

17. The compound of claim 14 in which the linker is trivalent.

18. The compound of claim 17 in which R3′ is of the form —CH2-CH(—R3′Rem2)-R3′Rem1, and R3′Rem1 and R3′Rem2 are independently chosen organic moieties consisting of at least one primarily alkyl moiety and, optionally, one or more spacers.

19. The compound of claim 17 in which R3′ is of a form selected from the group consisting of
—CH2-CH(—R3b)-(spacerA1)-(spacerA2)-R3″
—CH2-CH(—R3b)-(spacerA)-R3″
—CH2-CH(-(spacerB)-R3b)-(spacerA1)-(spacerA2)-R3″
—CH2-CH(-(spacerB)-R3b)-(spacerA)-R3″
—CH(—R3b)-(spacer A1)-(spacerA2)-R3″
—CH(—R3b)-(spacerA)-R3″
—CH(-(spacerB)-R3b)-(spacer A1)-(spacerA2)-R3″
—CH(-(spacerB)-R3b)-(spacerA)-R3″
where each of spacerA, spacer A1, spacerA2 and spacerB is independently chosen, and R3″ and R3b are primarily alkyl moieties.

20. The compound of claim 18 in which SpacerA1 is —NH— or —O—, Spacer A2 is —C(═O)—, SpacerA is —O—, and SpacerB is —O—.

21. The compound of claim 1 in which condition (4) applies.

22. The compound of claim 19 in which condition (4)(a) applies.

23. The compound of claim 22 in which R4 is hydrogen, -(primarily alkyl), or -(spacer)-(primarily alkyl).

24. The compound of claim 21 in which condition 4(b) applies.

25. The compound of claim 24 in which R4 is -(primarily alkyl), or -(spacer)-(primarily alkyl).

26. The compound of claim 21 in which condition (4)(c) applies.

27. The compound of claim 26 in which the organic moieties of R1 and R4 are both primarily alkyl moieties.

28. The compound of claim 1 in which condition (5) applies.

29. The compound of claim 28 wherein the organic moiety within the group A as defined by (5) is a primarily alkyl moiety.

30. The compound of claim 29 wherein the organic moiety within the group A as defined by (5) is strongly lipophilic.

31. A non-naturally occurring, biologically active compound of the form R—O-Z, where R is an organic moiety comprising a carbohydrate moiety, and Z is an organic moiety comprising a steroidal, terpenoidal or alkaloidal moiety.

32. The compound of claim 31 where Z comprises a steroidal moiety.

33. A non-naturally occurring, biologically active compound which comprises a Pet unit, 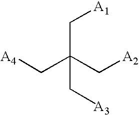
the arms of which are denoted as A1-A4, wherein
(1) one arm of the Pet unit is connected to the 0-1 atom of a ceramide and the other arms are connected to hydrogen or an organic moiety; or
(2) one arm of the Pet unit is a —CH2-NH— arm and is connected to an organic moiety consisting of at least one primarily alkyl moiety and optionally one or more spacers, a second arm is a —CH2-Ch- arm and is connected to an organic moiety consisting of at least one primarily alkyl moiety and optionally one or more spacers, and the remaining arms are connected to hydrogen, or an organic moiety,
with the caveat that the compound does not comprise a phosphate equivalent.

34. The compound of claim 33 where (1) applies.

35. The compound of claim 33 where (2) applies.

36. A non-naturally occurring, biologically active compound defined by the general formula F-AF: 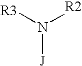
where R2 is hydrogen or an organic moiety; J is an organic moiety comprising at least one sugar unit and/or at least one Pet (pentaerythritol) unit; R3 is of the form -(Z)₀₋₁-CF2-R3′, Z is a single spacer, -spacer-CH2-spacer-, or a spacer cluster, and R3′ is a primarily alkyl moiety.

37. The compound of claim 36 where there is one Z.

38. The compound of claim 37 where it is a single spacer.

39. The compound of claim 38 where Z is —C(═O)—.

40. The compound of claim 36 where R3′ is strictly alkyl.

41. The compound of claim 36 where more than one carbon atom is fluorinated.

42. The compound of claim 36 where all of the alkanyl carbon atoms of R3′ are fluorinated.

43. A non-naturally occurring, biologically active series A compound represented by the following general formula F-1A: 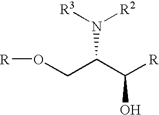
where R comprises a carboydrate moiety; R1 is primarily alkyl or -(spacer)-primarily alkyl; R2 is hydrogen, primarily alkanyl, or -(spacer)-primarily alkanyl; and R3 is
(A) -Z-R3″, where Z is a linker moiety consisting of one or more alkyl moieties and/or one or more spacers; and R3″ is a polyunsaturated moiety or an organic moiety comprising a steroidal moiety; or
(B) -Z-CF2-R3″, where Z is a linker moiety consisting of one or more alkyl moieties and/or one or more spacers; and R3″ is primarily alkanyl, or
(C) -Z(-R3b)-R3″, where Z is a trivalent linker moiety consisting of one or more alkyl moieties, including at least one secondary carbon, and/or one or more spacers; where R3b and R3″ are the same or different primarily alkyl moieties.

44. The compound of claim 43 where if R1 contains non-alkyl moieties, they are hydroxyl moieties.

45. The compound of claim 43 in which R2, if organic, is —CH2-R2′ or —(C═O)—R2′, where R2′ is primarily alkanyl

46. The compound of claim 43 in which, in R3, Z is a single spacerF, or is of the form spacerF-Z′-spacerL, where spacerF is the first spacer in Z, spacerL is the last spacer in Z, and Z′ is the remainder of Z, if any, and may comprise one or more spacers.

47. The compound of claim 46 in which SpacerF is —C(═O)—, and SpacerL is —O— or —C(═O)—.

48. The compound of claim 46 in which Z is —C(═O)—, —C(═O)—CH2-CH(—O—)—, or —C(═O)—CH(—NH—C(═O)—)—CH2-O—.

49. The compound of claim 43 in which
R¹ is a substitution group selected from the group consisting of
—CH₂(CH₂)ᵢCH₃,
—CH═CH(CH₂)ᵢCH₃,
—CH(OH)(CH₂)ᵢCH₃,
—CH₂(CH₂)ᵢCH(CH₃)CH₂CH₃, and
—CH(OH)(CH₂)ᵢCH(CH₃)₂, wherein i is an integer with values from 6 to 20; and
R² is a substitution group selected from the group consisting of
—H,
—CH₂(CH₂)ⱼCH₃, and
—CO(CH₂)ⱼCH₃, wherein j is an integer with values from 0 to 30.
R³ is a substitution group selected from the group consisting of
—CO(CF₂)ₘCF₃,
—COCF₂(CH₂)ₘCH₃,
—CO(CH₂)ₖ(CH═CHCH₂)₂(CH═CHCH₂)ₙ(CH₂)ₘCH₃, 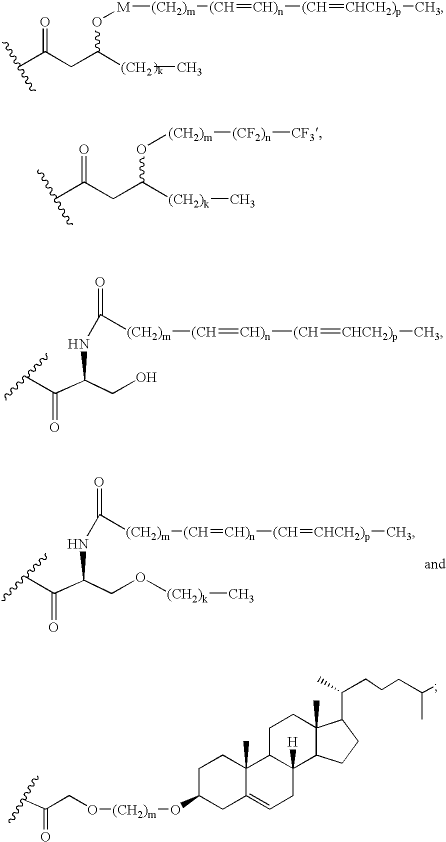
wherein M is CH₂ or CO; k and m are independent integers with values from 0 to 30, and n and p are independent integers with values from 0 to 10.

50. The compound of claim 49, further defined by the following structure: 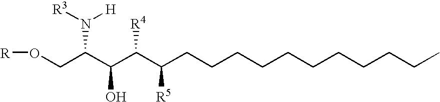
wherein R is chosen from structure I or II, 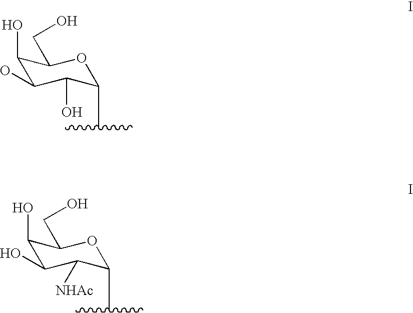
R⁴ is H or OH, and R⁵ is H; or R⁴ and R⁵ form a double bond.

51. The compound of claim 50, having the structure

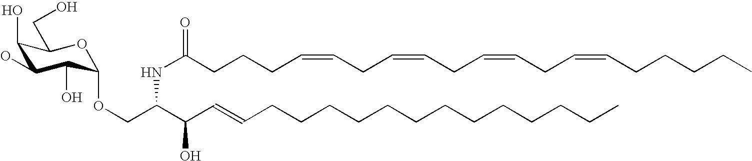

52. A non-naturally occurring, biologically active compound having the following formula F-4B: 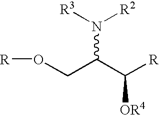
wherein R comprises a carbohydrate moiety;
R1 is hydrogen or -Z1-R1′, where Z1 is a linker moiety consisting of one or more spacers and, optionally, one or more alkanyl moieties; and where R1′ is primarily alkyl;
R2 is hydrogen, primarily alkanyl, or -(spacer)-primarily alkanyl;
R3 is -Z3-R3′, where Z3 is a linker moiety consisting of one or more alkanyl moieties and/or one or more spacers; and where R3′ is primarily alkyl, or is an organic moiety comprising a steroidal moiety; and
R4 is hydrogen or -Z4-R4′, where Z4 is a linker moiety consisting of one or more alkanyl moieties and/or one or more spacers; and where R4′ is primarily alkanyl.

53. The compound of claim 52 in which Z1 is —X—Y-Z, where X and Z are independently —CH2- or —C(═O)-, and Y is —O—, —NH—, or —S—.

54. The compound of claim 52 in which, if R1′ contains non-alkyl moieties, they are hydroxyl moieties.

55. The compound of claim 52 where R2, if organic, is —CH2-R2′ or —(C═O)—R2′, where R2′ is primarily alkanyl.

56. The compound of claim 52 in which R3 is at least partially fluorinated, or comprises a polyunsaturated moiety, or comprises a steroidal moiety.

57. The compound of claim 52 in which Z3 is a single spacerF, or is of the form spacerF-Z3′-spacerL, where spacerF is the first spacer in Z3, spacerL is the last spacer in Z3, and Z3′ is the remainder of Z3, if any, and may comprise one or more spacers.

58. The compound of claim 57 in which SpacerF is —C(═O)—and SpacerL is —O— or —C(═O)—.

59. The compound of claim 58 in which Z3 is —C(═O)—, —C(═O)—CH2-CH(—O—)—, or —C(═O)—CH(—NH—C(═O)—)—CH2-O—.

60. The compound of claim 52 in which Z4 is —CH2- or —C(═O)—.

61. The compound of claim 52 in which if R4 contains non-alkyl moieties, they are hydroxyl moieties.

62. The compound of claim 52 which is a compound of series BBB, where
R¹ is a substitution group selected from the group consisting of
—H,
—X—Y-Z-(CH₂)ᵢCH₃,
—X—Y-Z-(CH₂)ᵣ(CH═CHCH₂)_{q}(CH₂)ᵢCH₃, and
—X—Y-Z-(CH₂)ᵣCH(OH)(CH₂)ᵢCH₃,
wherein X and Z are independently CH₂ or CO, and Y is O, NH, or S; i and r are independent integers with values from 0 to 30, and q is an integer with values from 1 to 10;
R² is a substitution group selected from the group consisting of
—H,
—CH₂(CH₂)ⱼCH₃, and
—CO(CH₂)ⱼCH₃, wherein j is an integer with value from 0 to 30;
R³ is a substitution group selected from the group consisting of
—CO(CH₂)ₘCH(OH)(CH₂)ₖCH₃
—CO(CF₂)ₘCF₃,
—COCF₂(CH₂)ₘCH₃,
—CO(CH₂)ₖ(CH═CHCH₂)ₙ(CH₂)ₘCH₃, and
a structure of the following: 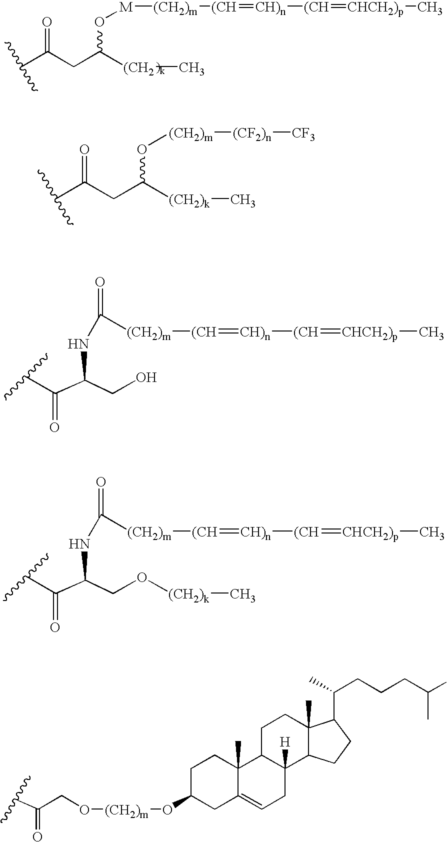
wherein M is CH₂ or CO; k and m are independent integers with values from 0 to 30, and n and p are independent integers with values from 0 to 10; and
R⁴ is a substitution group selected from the-group consisting of
—H,
-M-(CH₂)ₛCH(OH)(CH₂)ₜCH₃, and
-M-CH(CH₂OH)(CH₂)CH₃
wherein M is CH₂ or CO; and s and t are independent integers with values from 0 to 30.

63. The compound of claim 62, further defined by the following structure: 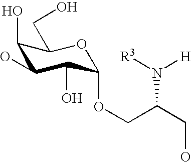
where R3 is as previously defined

64. The compound of claim 63 where the R3 therein has the structure

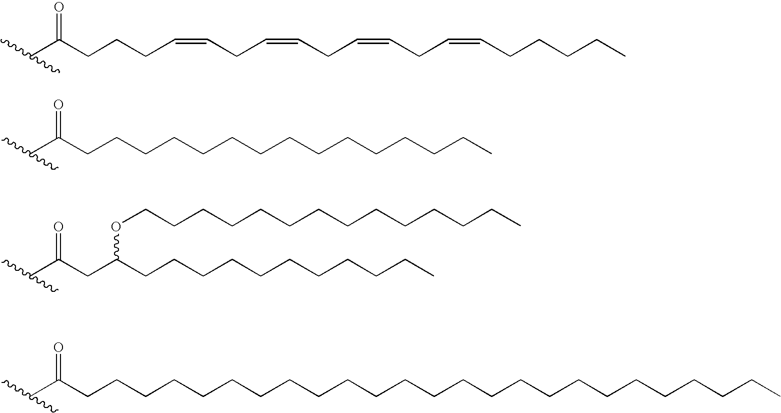

65. The compound of claim 64 which has the structure

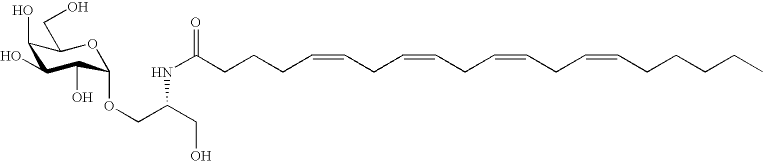

66. A non-naturally occurring, biologically active compound which is a series C compound having the following general formula F-8C 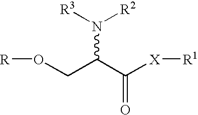
wherein R comprises a carbohydrate moiety; R1 is hydrogen or is an organic moiety which is substantially linear and primarily alkyl; X denotes —O—, —NH— or —S—; R2 is hydrogen, primarily alkanyl, or -(spacer)-primarily alkanyl; and R3 is -Z3-R3′, where Z3 is a linker moiety consisting of one or more alkyl moieties and/or one or more spacers; and where R3′ is primarily alkyl, or is an organic moiety comprising a steroidal moiety.

67. The compound of claim 66 where, if R1 contains non-alkyl moieties, they are hydroxyl moieties.

68. The compound of claim 66 where R2, if organic, is —CH2-R2′ or —(C═O)—R2′, where R2′ is primarily alkanyl.

69. The compound of claim 66 where R3 is at least partially fluorinated, or comprises a polyunsaturated moiety, or comprises a steroidal moiety.

70. The compound of claim 66 where Z3 is a single spacerF, or is of the form spacerF-Z3′-spacerL, where spacerF is the first spacer in Z3, spacerL is the last spacer in Z3, and Z3′ is the remainder of Z3, if any, and may comprise one or more spacers.

71. The compound of claim 70 in which SpacerF is —C(═O)— and SpacerL is preferably —O— or —C(═O)—.

72. The compound of claim 70 in which Z3 is —C(═O)—, —C(═O)—CH2-CH(—O—)—, or —C(═O)—CH(—NH—C(═O)—)—CH2-O—.

73. The compound of claim 66 which is a series CCC compound in which
R¹ is a substitution group selected from the group consisting of
—H,
—(CH₂)ᵣ(CH═CHCH₂)_{q}(CH₂)ᵢCH₃, and
—(CH₂)ᵣCH(OH)(CH₂)ᵢCH₃,
wherein r and i are independent integers with values from 0 to 30, and q is an integer with values from 0 to 10,
R² preferably is a substitution group selected from the group consisting of
—H,
—CH₂(CH₂)CH₃, and
—CO(CH₂)CH₃,
wherein j is an integer with values from 0 to 30,
R³ is a substitution group selected from the group consisting of
—CO(CH₂)ₘCH(OH)(CH₂)ₖCH₃
—CO(CF₂)ₘCF₃,
—COCF₂(CH₂)ₘCH₃,
—CO(CH₂)ₖ(CH═CHCH₂)ₙ(CH₂)ₘCH₃, and
a structure of the following: 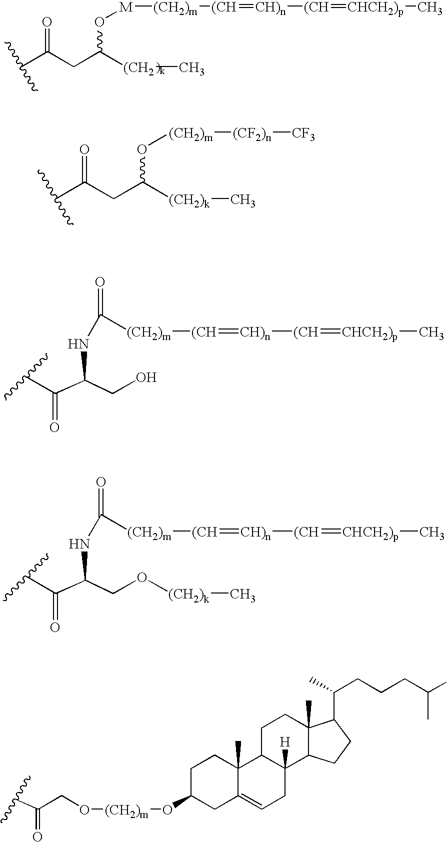
wherein M is CH₂ or CO; k and m are independent integers with values from 0 to 30, and n and p are independent integers with values from 0 to 10.

74. The compound of claim 73, further defined by the following: 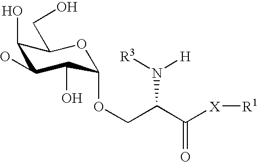
wherein R1, R3 and X are as previously defined.

75. A non-naturally occurring, biologically active compound which is a series D compound having the general structure F-10D: 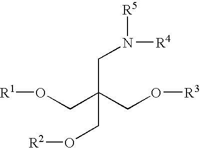
wherein R¹ and R² is are independently selected from the group consisting of hydrogen, an organic moiety comprising a carbohydrate moiety, and an organic moiety comprising another Pet unit, and at least one of R¹ and R² is not hydrogen; R3 is a substantially linear and primarily alkyl moiety; R4 is hydrogen, or a substantially linear, primarily alkanyl moiety; and R5 is -Z5-R5′, where Z5 is a linker moiety consisting of one or more alkyl moieties and/or one or more spacers; and where R5′ is primarily alkyl, or is an organic moiety comprising a steroidal moiety.

76. The compound of claim 75 where, if R3 contains non-alkyl moieties, they are hydroxyl moieties.

77. The compound of claim 75 where R4, if organic, is —CH4-R4′ or —(C═O)—R4′, where R4′ is primarily alkanyl.

78. The compound of claim 75 where R5 is at least partially fluorinated, or comprises a polyunsaturated moiety, or comprises a steroidal moiety.

79. The compound of claim 75 where Z5 is a single spacerF, or is of the form spacerF-Z5′-spacerL, where spacerF is the first spacer in Z5, spacerL is the last spacer in Z5, and Z5′ is the remainder of Z5, if any, and may comprise one or more spacers.

80. The compound of claim 79 where SpacerF is —C(═O)—and SpacerL is —O— or —C(═O)—.

81. The compound of claim 75 where Z5 is —C(═O)—, —C(═O)—CH2-CH(—O—)—, or —C(═O)—CH(—NH—C(═O)—)—CH2-O—.

82. The compound of claim 75 which is a series DDD compound, where
R³ is a substitution group selected from the group consisting of
—H,
—(CH₂)ᵥCH₃,
—CO(CH₂)ᵥCH₃,
—CO(CH₂)ᵤ(CH═CHCH₂)ᵥ(CH₂)ₜCH₃,
—(CH₂)ᵤCH(OH)(CH₂)ₜCH₃, and
—CO(CH₂)ᵤCH(OH)(CH₂)ₜCH₃,
wherein t and u are independent integers with values from 0 to 30, and v is an integer with values from 1 to 10.
R⁴ is a substitution group selected from the group consisting of
—H,
—CH₂(CH₂)ₛCH₃, and
—CO(CH₂)ₛCH₃ wherein s is an integer with values from 0 to 30.
R⁵ is a substitution group selected from the group consisting of
—CO(CH₂)ₘCH₃,
—CO(CH₂)ₘCH(OH)(CH₂)ₖCH₃,
—CO(CF₂)ₘCF₃,
—COCF₂(CH₂)ₘCH₃,
—CO(CH₂)ₖ(CH═CHCH₂)ₙ(CH₂)ₘCH₃, and
a structure of the following: 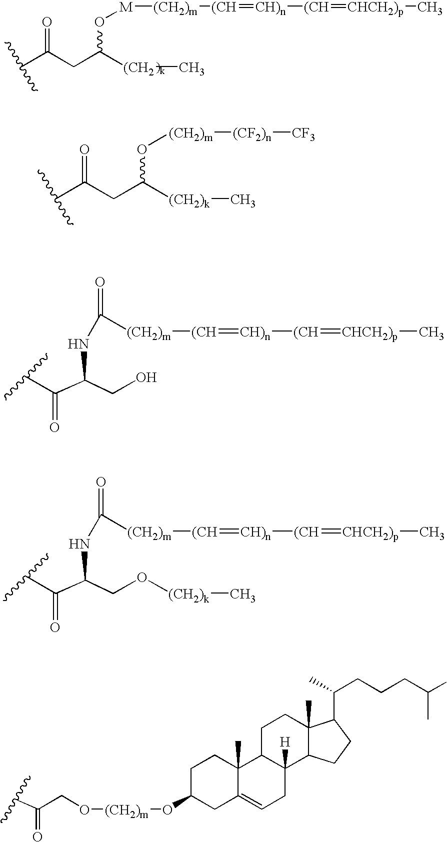
wherein M is CH₂ or CO; k and m are independent integers with values from 0 to 30, and n and p are independent integers with values from 0 to 10.

83. The compound of claim 82, further defined by the following: 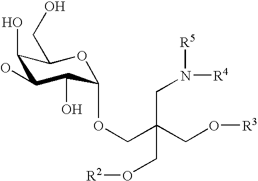
wherein
R² is hydrogen or α-D-galactopyranosyl residue (I), 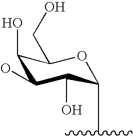
and R3, R4 and R5 are as previously defined.

84. A non-naturally occurring, biologically active compound which is a series E compound defined by the following structure F-12E: 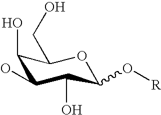
wherein R is a residue of a steroid, terpenoid, or an alkaloid.

85. The compound of claim 84 where R is a residue of a terpenoid.

86. The compound of claim 85 where the terpenoid is a monoterpenoid, sesqiterpenoid, diterpenoid, or triterpenoid.

87. The compound of claim 84 where R is a residue of a steroid.

88. The compound of claim 87 where R is selected from the group consisting of:

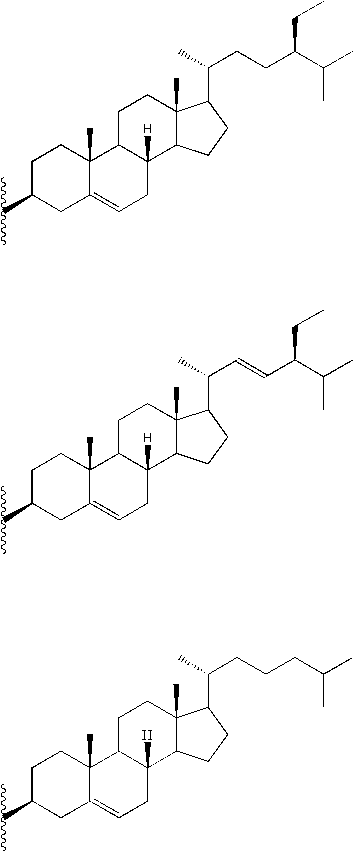

89. The compound of claim 84 where R is the residue of an alkaloid.

90. The compound of claim 89 where the alkaloid is an immunomodulatory alkaloid.

91. The compound of claim 89 where the alkaloid is an antitumor alkaloid.

92. The compound of claim 1 where the carbohydrate moiety is a monosaccharide.

93. The compound of claim 1 where said carbohydrate moiety comprises at least one sugar unit which is hexosyl, pentosyl, or nonosyl.

94. The compound of claim 93 in which each sugar unit is hexosyl, pentosyl or nonosyl.

95. The compound of claim 94 in which each sugar unit is (a) galactose, glucose, mannose or fucose, (b) a deoxy or N-acetyl derivative of (a), of (c) a sialic acid.

96. The compound claim 1 where the inner sugar unit is galactose.

97. The compound of claim 96 where the inner sugar unit is alpha-galactose.

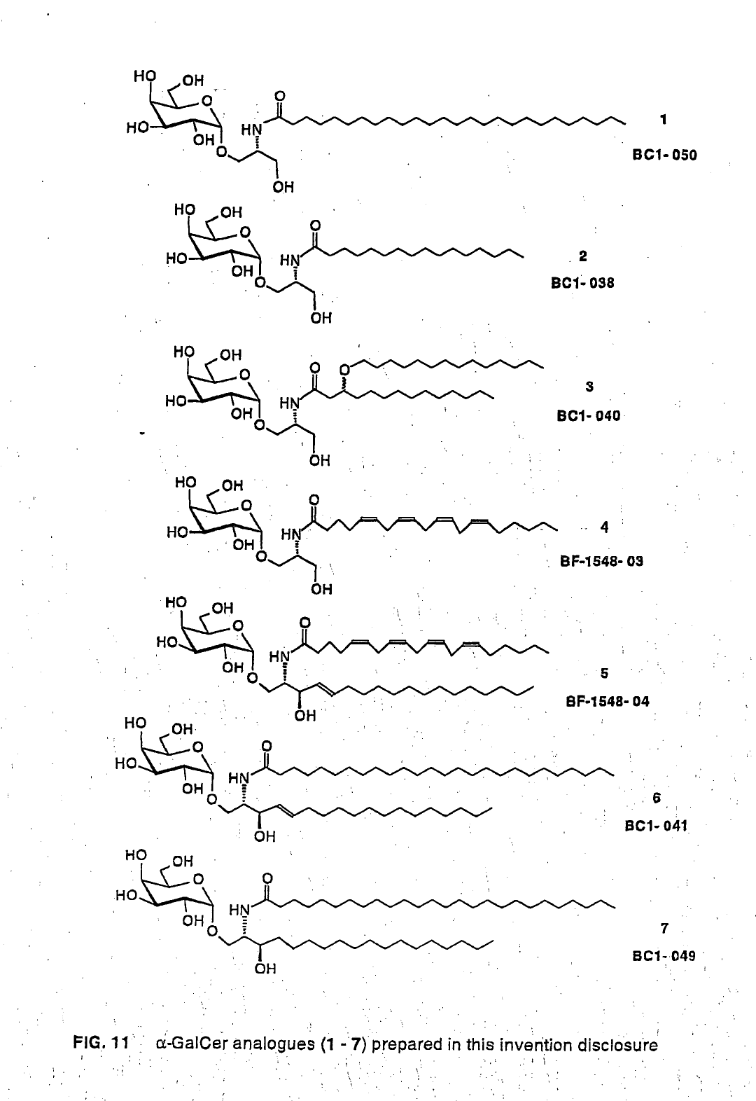

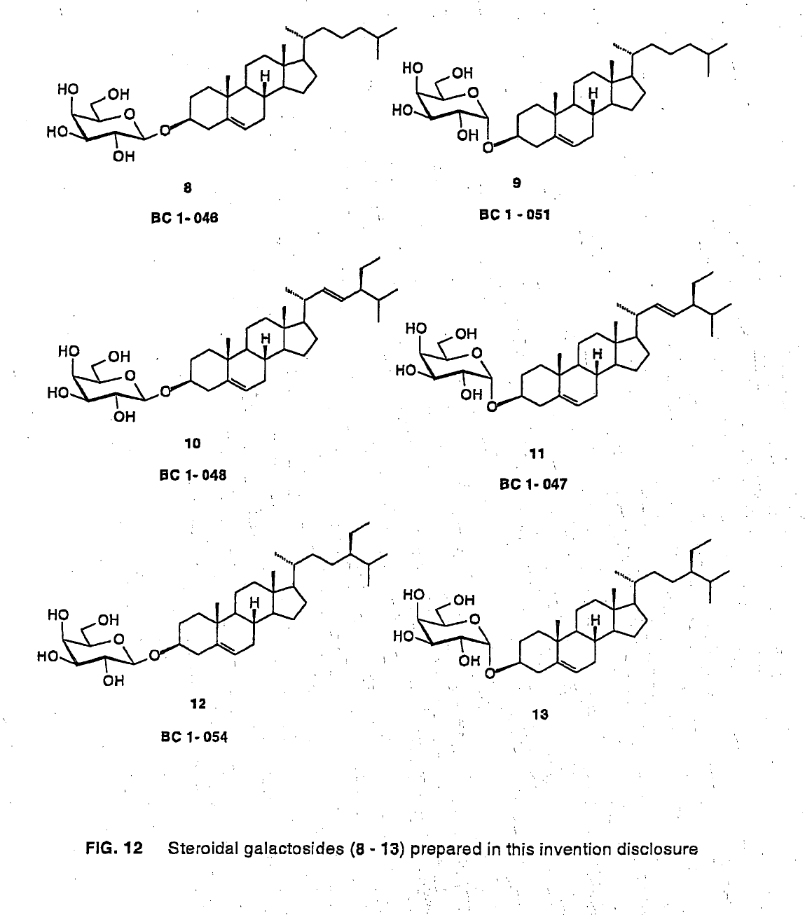

98. A compound selected from the group consisting of compounds 1-5 in FIG. 11, 8-13 in FIG. 12, and 033 in FIG. 31.

99. A pharmaceutically acceptable composition comprising at least one compound according to claim 1.

100. The composition of claim 99, where said compound has immodulatory activity, and further comprising at least one immunomodulatory agent which is not one of said compounds.

101. The composition of claim 100, where at least one such immunomodulatory agent is an immunogen.

102. The composition of claim 100, where at least one such immunomodulatory agent is an adjuvant.

103. The composition of claim 102, where said adjuvant is selected from the group consisting of lipid A, lipid A analogues, CpG-containing oligonucleotides, muramyl dipeptides, sitosterols, alum, and QS-21.

104. The composition of claim 99, further comprising at least one antiviral, antibacterial, antiparasitic or antitumor agent other than said compound.

105. The composition claim 99, in liposomal form.

106. (canceled)

107. A method of protecting a mammalian subject against a virus, microbial infection, parasite or cancer which comprises administering to the subject a pharmaceutically effective amount of a compound according to claim 1 which has pharmaceutical activity against such virus, microbial infection, parasite, or cancer.

108. The method of claim 107 wherein protection is against a virus.

109. The method of claim 108 wherein said virus is HIV-1.

110. The method of claim 107 wherein protection is against a cancer.

111. The method of claim 110 which further comprises administration of an immunogen comprising a tumor-associated epitope.

112. The method of claim 111 where said immunogen comprises a MUC1 epitope.

113. The method of claim 111 where said immunogen comprises a Tn, TF, sialyl Tn, sialylTF, F1-α, Globo H, Fucosyl GM1, or GalNAc GM1 epitope.

114. The method of claim 110 wherein said cancer is a melanoma.

115. The method of claim 107 wherein protection is against a microbial infection.

116. The method of claim 115 wherein the microbial infection is a malaria infection.

117. The method of claim 115 wherein the microbial infection is a tuberculosis infection.

118. A method of protecting a subject against an immune disase or an inflammation which comprises administering an immunoinhibitory amount of a compound according to claim 1.

119. The method of claim 118 where said protection is against an autoimmune disease.

120. The method of claim 119 wherein said autoimmune disease is diabetes.

121. The method of claim 119 wherein said autoimmune disease is asthma, eczema, multiple sclerosis or rheumatoid arthritis.

122. The method of claim 118 where said protection is against inflammation.

123. The method of claim 107 further comprising administering a pharmaceutically effective amount of at least one immunomodulatory agent which is not one of said compounds.

124. The method of claim 123, where at least one such immunomodulatory agent is an immunogen.

125. The method of claim 123, where at least one such immunomodulatory agent is an adjuvant.

126. The method of claim 125, where said adjuvant is selected from the group consisting of lipid A, lipid A analogues, CpG-containing oligonucleotides, muramyl dipeptides, sitosterols, alum, and QS-21.

127. The composition of claim 107, further comprising a pharmaceutically effective amount of at least one antiviral, antibacterial, antiparasitic or antitumor agent other than said compound.

128. The compound claim 1 which has immunostimulatory activity.

129. A method of stimulating the immune system of a mammalian subject which comprises administering to said subject an immunostimulatory amount of the compound of claim 128.

130. The method of claim 129 which further comprises administering to the subject an immunologically effective amount of an immunogen, the immune response to said immunogen being enhanced by said compound.

131. The method of claim 130 in which the immunogen is a disease-associated immunogen and the subject suffers from that disease.

132. The method of claim 131 in which the immunogen is a tumor-associated immunogen.

133. The method of claim 130 in which the immunogen comprises a carbohydrate epitope.

134. The method of claim 133 in which the immunogen comprises a Tn, TF or sialyl-Tn epitope.

135. The method of claim 130 in which the immunogen comprises a peptide epitope.

136. The method of claim 135 in which the immunogen comprises a MUC1 epitope.

137. The method of claim 129 in which the compound is delivered by means of a liposomal formulation.

138. The method of claim 129 in which the immunogen comprises a strongly lipophilic group.

139. The method of claim 129 in which the immunogen is delivered by means of a liposomal formulation.

140. A galactosyl donor illustrated by the following structure: 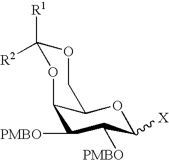
wherein X represents a leaving group including, but not limited to, halogen, —OC(NH)CCl₃, —SR, SO₂R, —O(CH₂)₃CH═CH₂, —P(OR)₂, and —P(O)(OR)₂ wherein R is an alkyl or aromatic group.

141. A process of making an α-GalCer analogue comprising an aglycon, said aglycon comprising at least one double bond, which comprises the following steps:
a) carrying out a glycosylation reaction, in the presence of a Lewis acid as a catalyst, by using the following glycosyl donor: 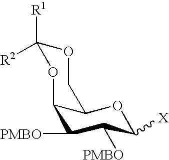
wherein
X represents a leaving group including, but not limited to, halogen, —OC(NH)CCl₃, —SR, SO₂R, —O(CH₂)₃CH═CH₂, —P(OR)₂, and P(O)(OR)₂, wherein R is an alkyl or aromatic group;
R¹ and R² are independently hydrogen atom, alkyl group, or aromatic group;
and the following glycosyl acceptor: 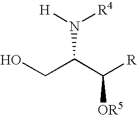
wherein
R³ is hydrogen, or an alkyl or alkenyl group, substituted or unsubstituted;
R⁴ is an amine protecting group or an fatty acyl group; and
R⁵ is a hydroxyl protecting group;
to provide the following glycoside: 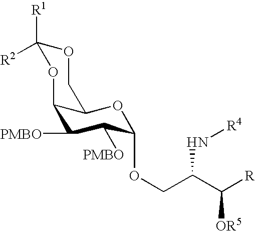
wherein R¹ to R⁵ are defined as above;
b) removing the amine protecting group R⁴, when applicable, in the product formed in step a), to give the following free amine: 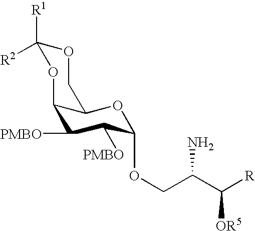
wherein
R¹ to R⁵ are defined as above;
c) introducing a fatty acyl group at the amine position of the product formed in step b), in the presence of a conventional coupling reagent, to give: 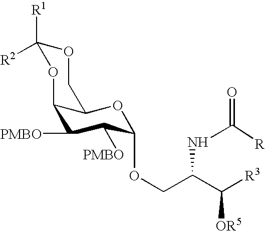
whereinR is an alkyl or alkenyl group, substituted or unsubstituted, and R¹ to R⁵ are defined as above;
d) deprotecting the protecting groups R⁵, PMB, and R¹R²CH acetal/ketal at 4,6-O-position in the product formed in step c) are deprotected in a non-preferential order to give the α-GalCer analogue of the following structure: 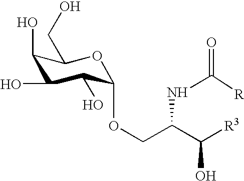
wherein R and R³ are independently alkyl groups, with at least one group carrying at least one double bond.

144. The method of claim 143 in which step (d) is carried out, with respect to at least one of the protecting groups (R⁵, PMB and R¹R²CH acetal/ketal), before step b)

145. The compound of claim 1 which has a molecular weight of less than 10,000 daltons

146. The compound of claim 145 which has a molecular weight less than 5,000 daltons.

147. The compound of claim 145 which has a molecular weight less than 2,500 daltons

148. The compound of claim 145 which has a molecular weight less than 1,000 daltons.

149. (canceled)
